(19) United States
(12) Patent Application Publication
(10) Pub. No.: US 2018/0044428 A1
(43) Pub. Date: Feb. 15, 2018

(54) COMPOSITIONS AND METHODS FOR ENHANCING THE EFFICACY OF CANCER THERAPY

(71) Applicant: PROVIDENCE HEALTH & SERVICES-OREGON, PORTLAND, OR (US)

(72) Inventors: MICHAEL GOUGH, PORTLAND, OR (US); MARKA CRITTENDEN, PORTLAND, OR (US)

(21) Appl. No.: 15/556,864

(22) PCT Filed: Mar. 9, 2016
(86) PCT No.: PCT/US2016/021486

§ 371 Date: Sep. 8, 2017

Related U.S. Application Data
(60) Provisional application No. 62/131,562, filed on Mar. 11, 2015.

(51) Int. Cl. C07K 16/28; A61N 5/10; C07K 14/705; C07K 16/24; A61K 39/395; A61K 31/7068

(52) U.S. Cl. CPC C07K 16/2878; A61K 39/39558; C07K 16/2818; A61K 31/7068; C07K 14/70578; C07K 16/247; A61N 5/10; A61K 2039/545

(57) ABSTRACT

The present invention features compositions and methods for enhancing an anti-tumor response by administering an OX40 agonist (e.g., an anti-OX40 antibody) and/or an anti-CTLA4 antibody (e.g., a CTLA4-blocking antibody) in combination with a cancer therapy.

### BACKGROUND OF THE INVENTION

It is estimated that the one-year survival rate for all stages of pancreatic cancer is about 20%, while the five-year rate is as low as 6%. Contributing to these low survival rates is the fact that at time of diagnosis many patient have tumors that have already spread beyond the pancreas and metastasized to the point where surgical resection is impossible.

Recent studies have reported that decreased T cell infiltrate alone or in combination with increased macrophage infiltrate correlates with decreased survival in a variety of cancers, including patients with pancreatic cancer. In these retrospective studies, the patients had been treated with conventional cancer therapies, including chemotherapy, radiation and surgical resection, suggesting that the T cell and macrophage infiltrate in the tumor influences outcome in response to conventional therapies.

Over the past several years, there has been a surge of interest in immunotherapy as a novel adjunct to traditional cytotoxic oncologic therapies. With the clinical success of checkpoint inhibitors, such as Ipilimumab in melanoma, there is a broadened interest in applying immunotherapy to a larger spectrum of malignancies. With increasing clinical indications, combined modality therapy utilizing immunotherapy together with radiation or chemotherapy is more common. However, while combinatorial use is becoming more prevalent, there are few studies designed to optimize therapeutic efficacy based on timing of administration of each agent (Dewan et al., Clinical cancer research : an official journal of the American Association for Cancer Research, 2009. 15(17): 5379-88). Methods for increasing survival by improving response to conventional cancer therapies are therefore urgently required.

### SUMMARY OF THE INVENTION

As described below, the present invention features compositions and methods for enhancing an anti-tumor response by administering an OX40 agonist (e.g., an anti-OX40 antibody) and an anti-CTLA4 antibody (e.g., a CTLA4-blocking antibody) in combination with a chemotherapeutic agent and/or regimen. The invention is based at least in part on the discovery that such combinations of agents are particularly effective for treating tumors that are highly resistant to conventional treatment regimens (e.g., pancreatic tumors). Thus, the present invention provides immunotherapeutic compositions comprising an OX40 agonist and anti-CTLA4 antibody, and methods of administering an OX40 agonist and anti-CTLA4 in combination with a cancer therapy (e.g., chemotherapy and/or radiotherapy) for the treatment of cancer (e.g., pancreatic cancer).

In one aspect, the disclosure herein provides a method of enhancing chemotherapy or radiotherapy efficacy in a subject having a tumor, the method comprising administering to a subject an OX40 agonist and an anti-CTLA4 antibody before, during or after chemotherapy or radiotherapy.

In another aspect, the disclosure herein provides a method of treating a subject having a tumor, the method comprising: (a) administering to the subject an OX40 agonist and an anti-CTLA4 antibody; (b) obtaining a measurement of cells that indicates a reduction in macrophage differentiation in the subject; and (c) administering chemotherapy or radiotherapy to the subject.

In a further aspect, the disclosure herein provides a method of treating a subject having a tumor, the method comprising: (a) administering to the subject an OX40 agonist and an anti-CTLA4 antibody; (b) obtaining a measurement of cells that indicates a reduction in macrophage differentiation in the subject; and (c) administering an anti-IL4 antibody and chemotherapy or radiotherapy to the subject.

In yet another aspect, the disclosure herein provides a method of treating a subject having a tumor, the method comprising: (a) administering to the subject an OX40 agonist and an anti-CTLA4 antibody; obtaining a measurement of cells that indicates a reduction in macrophage differentiation in the subject; (b) administering chemotherapy to the subject; (c) administering to the subject an OX40 agonist and an anti-CTLA4 antibody; and (d) administering chemotherapy or radiotherapy to the subject.

In yet another aspect, the disclosure herein provides a method of enhancing chemotherapy or radiotherapy efficacy in a subject having a colorectal cancer, the method comprising administering to a subject an anti-CTLA4 antibody before, during or after chemotherapy or radiotherapy.

In yet another aspect, the disclosure herein provides a method of treating a subject having a colorectal tumor, the method comprising: (a) administering to the subject an anti-CTLA4 antibody; and (b) administering radiotherapy to the subject.

In yet another aspect, the disclosure herein provides a method of enhancing chemotherapy or radiotherapy efficacy in a subject having a colorectal cancer, the method comprising administering to a subject an OX40 agonist during or after chemotherapy or radiotherapy.

In yet another aspect, the disclosure herein provides a method of treating a subject having a colorectal cancer, the method comprising: (a) administering radiotherapy to the subject; and (b) administering to the subject an OX40 agonist.

In various embodiments of any aspect delineated herein, the anti-CTLA4 antibody is one or more of 9D9 and tremelimumab. In various embodiments of any aspect delineated herein, the chemotherapy or radiotherapy is administered about 1, 2, 3, 4, 5, 6, or 7 days after administration of the anti-CTLA4 antibody. In various embodiments of any aspect delineated herein, the chemotherapy or radiotherapy is administered about 1, 2, 3, or 4 days before administration of the anti-CTLA4 antibody.

In various embodiments of any aspect delineated herein, the OX40 agonist is an anti-OX40 antibody. In various embodiments, the anti-OX40 antibody is one or more of OX86, humanized anti-OX40 antibody, and 9B12. In various embodiments, the OX40 agonist is an OX40 fusion protein. In various embodiments of any aspect delineated herein, the OX40 agonist is administered about 1 or 2 days after administration of chemotherapy or radiotherapy.

In various embodiments of any aspect delineated herein, the method delays or reduces tumor growth, reduces tumor size, and/or enhances survival in the subject. In certain embodiments, the subject has a colorectal tumor.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “OX40 polypeptide” is meant a member of the TNFR-superfamily of receptors that is expressed on the surface of antigen-activated mammalian CD4⁺ and CD8⁺T lymphocytes. See, for example, Paterson et al., Mol Immunol 24, 1281-1290 (1987); Mallett et al., EMBO J 9, 1063-1068 (1990); and Calderhead et al., J Immunol 151, 5261-5271 (1993)). OX40 is also referred to as CD134, ACT-4, and ACT35. OX40 receptor sequences are known in the art and are provided, for example, at GenBank Accession Numbers: AAB33944 or CAE11757.

An exemplary human OX40 sequence is provided below:

| (SEQ ID NO: 91) | |
|---|---|
| 1 | mcvgarrlgr gpcaallllg lglstvtglh cvgdtypsnd rcchecrpgn gmvsrcsrsq |
| 61 | ntvcrpcgpg fyndvvsskp ckpctwcnlr sgserkqlct atqdtvcrcr agtqpldsyk |
| 121 | pgvdcapcpp ghfspgdnqa ckpwtnctla gkhtlqpasn ssdaicedrd ppatqpgetq |
| 181 | gpparpitvq pteawprtsq gpstrpvevp ggravaailg lglvlgllgp laillalyll |
| 241 | rrdqrlppda hkppgggsfr tpigeeqada hstlaki |

By “OX40 agonist” is meant an OX40 ligand that specifically interacts with and increases the biological activity of the OX40 receptor. Desirably, the biological activity is increased by at least about 10%, 20%, 30%, 50%, 70%, 80%, 90%, 95%, or even 100%. In certain aspects, OX40 agonists as disclosed herein include OX40 binding polypeptides, such as anti-OX40 antibodies (e.g., OX40 agonist antibodies), OX40 ligands, or fragments or derivatives of these molecules.

By “OX40 antibody” is meant an antibody that specifically binds OX40. OX40 antibodies include monoclonal and polyclonal antibodies that are specific for OX40 and antigen-binding fragments thereof. In certain aspects, anti-OX40 antibodies as described herein are monoclonal antibodies (or antigen-binding fragments thereof), e.g., murine, humanized, or fully human monoclonal antibodies.

By “CTLA4 polypeptide” is meant a polypeptide having at least 85% amino acid sequence identity to GenBank Accession No. AAL07473.1 or a fragment thereof having T cell inhibitory activity. The sequence of AAL07473.1 is provided below:

gi|15778586|gb|AAL07473.1|AF414120_1 CTLA4
[Homo sapiens]
(SEQ ID NO: 93)
MACLGFQRHKAQLNLATRTWPCTLLFFLLFIPVFCKAMHVAQPAVVLA
SSRGIASFVCEYASPGKATEVRVTVLRQADSQVTEVCAATYMMGNELT
FLDDSICTGTSSGNQVNLTIQGLRAMDTGLYICKVELMYPPPYYLGIG
NGTQIYVIDPEPCPDSDFLLWILAAVSSGLFFYSFLLTAVSLSKMLKK
RSPLTTGVYVKMPPTEPECEKQFQPYFIPIN

By “anti-CTLA4 antibody” is meant an antibody that selectively binds a CTLA4 polypeptide. Exemplary anti-CTLA4 antibodies include 9D9 and tremelimumab.

By “IL4 polypeptide” is meant a polypeptide having at least 85% amino acid sequence identity to NCBI Accession No. NP_000580 or a fragment thereof having immune cell (e.g., macrophage, T cell) differentiation activity. The sequence of NP_000580 is provided below:

gi|4504669|ref|NP_000580.1| interleukin-4
isoform 1 precursor [Homo sapiens]
(SEQ ID NO: 94)
MGLTSQLLPPLFELLACAGNFVHGHKCDITLQEIIKTLNSLTEQKTLC
TELTVTDIFAASKNTTEKETFCRAATVLRQFYSHHEKDTRCLGATAQQ
FHRHKQLIRELKRLDRNLWGLAGLNSCPVKEANQSTLENFLERLKTIM
REKYSKCSS

By “anti-IL4 antibody” is meant an antibody that selectively binds an IL4 polypeptide. 11B11 is an exemplary anti-IL4 antibody.

By “antibody” is meant an immunoglobulin molecule that recognizes and specifically binds a target. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (VL) and an antibody heavy chain variable region (VH), however, it does not necessarily have to comprise both. For example, a so-called Fd antibody fragment consists only of a VH domain, but still retains some antigen-binding function of the intact antibody.

By “ameliorate” is meant decrease, suppress, attenuate, diminish, arrest, or stabilize the development or progression of a disease.

By “antigen binding fragment” is meant a portion of an intact antibody that binds antigen. In particular, the term antigen binding fragment refers to the antigenic determining variable regions of an intact antibody. The antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

By “cancer” is meant a disease or disorder characterized by excess proliferation or reduced apoptosis. For example, the compositions and methods of the invention are useful for the treatment of pancreatic cancer.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “enhances” is meant a positive alteration of at least 10%, 25%, 50%, 75%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

A “variable region” of an antibody refers to the variable region of the antibody light chain or the variable region of the antibody heavy chain, either alone or in combination. The variable regions of the heavy and light chain each consist of four framework regions (FW) connected by three complementarity determining regions (CDRs) also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS, 1A-1C show adaptive immune remodeling of tumor macrophages. Immunocompetent C57BL/6 mice bearing Panc02 tumors were left untreated (NT) or were treated with 100 mg/kg gemcitabine intraperitoneally (i.p.) on days 14 and 17 (GZ), then tumors were harvested at day 21. Figure lA depicts two images showing immunohistology for F4/80⁺ macrophages (green) and DAPI (blue). Multiple images across the tumor were merged to generate a margin-to-margin overview of the entire tumor. Tumor margins are indicated by white arrows. FIG. 1B shows two scatter graphs. Immunocompetent C57BL/6 mice bearing day 14 Panc02 tumors were left untreated or were treated with 250 μg anti-OX40, 250 μg anti-CTLA4 or the combination. Tumors were harvested at day 4 or day 8 following immunotherapy and single cell suspensions were stained and sorted by flow cytometry, gating on graph in panel (i) CD11b⁺ cells and graph in panel (ii) Gr1^{lo}IA⁺ cells within the CD11b⁺ population. FIG. 1C provides an image of a Western blot showing sorted tumor macrophages that were lysed and western blotted for expression of Arginase I and GAPdH.

FIGS. 2A and 2B show that preparative immunotherapy improved chemotherapy. FIG. 2A depicts a linear graph (panel i) and a scatterplot (panel ii). Immunocompetent C57BL/6 mice bearing Panc02 tumors were left untreated or treated with 250 μg anti-OX40, 250 μg anti-CTLA4 or the combination on day 14 (red dashed line). On day 18 mice were randomized to no further treatment or twice weekly gemcitabine (100 mg/kg intraperitoneally) for 3 weeks. In FIG. 2A, panel (i), the graph shows mean tumor area for each group with 6-7 mice per group. In FIG. 2A, panel (ii), the graph shows tumor area on day 39 for groups receiving chemotherapy. Each symbol represents one animal. FIG. 2B provides five graphs (panels i-v) showing survival curves for mice treated as in FIG. 2A, comparing two groups at a time for clarity. Key: NS not significant; *p<0.05; **p<0.01; ***p<0.005; ****p<0.001).

FIG. 3 shows three scatter graphs depicting tumor infiltrating immune cells following preparative immunotherapy. Immunocompetent C57BL/6 mice bearing Panc02 tumors were left untreated or treated with 250 μg anti-OX40 and 250 μg anti-CTLA4 on day 14. Tumors were harvested on day 4, or 7 following treatment and analyzed for infiltrating cell populations by flow cytometry for CD3⁺CD8⁺T cells (panel (i)); CD3⁺CD4⁺T cells (panel (ii)); or CD11b⁺(panel (iii)), myeloid cells. Each symbol represents one tumor. Key: NS not significant; *p<0.05.

FIGS. 4A-4E show that combination therapy drives Type 2 helper T cell (Th2) differentiation. FIG. 4A shows immunocompetent C57BL/6 mice bearing Panc02 tumors that were left untreated or treated with 250 μg anti-OX40, 250 μg anti-CTLA4 or the combination on day 14. Lymph nodes were harvested 7 days later and analyzed by flow cytometry for cell populations. FIG. 4A depicts four graphs showing the number of CD4 T cells (panel (i)); CD4⁺FoxP3⁺T regulatory cells (panel (ii)); CD4⁺FoxP3⁻T cells (panel (iii)); and CD8 T cells (panel (iv)). FIG. 4B depicts four graphs showing examples of intracellular staining for the transcription factors Tbet and GATA3 in FoxP3⁻CD4⁺T cells from untreated mice (panel (i)) or mice treated with anti-CTLA4 (panel (ii)); anti-OX40 (panel (iii) or anti-CTLA4 and anti-OX40 (panel iv). FIG. 4C shows two graphs providing a summary of data as per FIG. 4B showing the proportion of FoxP3⁻CD4 T cells that are GATA3⁺Tbet⁻(panel (i)) or GATA3⁻Tbet⁺(panel (ii)). Each symbol represents 1 mouse. FIG. 4D describes lymph node cells harvested as in FIG. 4A that were stimulated in vitro with plate-bound anti-CD3 for 4 hours in the presence of secretion inhibitors. Cells were surface stained then intracellularly stained for cytokines. FIG. 4D provides two graphs showing the percentage of FoxP3⁻CD4 T cells that are IL-4⁺IFNγ⁻(panel (i)) or IL-4⁻IFNγ⁺(panel (ii)). FIG. 4E provides two graphs showing lymph node CD8 T cells harvested as in FIG. 4A that were intracellularly stained for the transcription factor Eomes (panel (i)) and stimulated as in FIG. 4D and stained for IFNγ(panel (ii)). Key: NS not significant; *p<0.05; **p<0.01; ***p<0.005; ****p<0.001).

FIGS. 5A and 5B show that interleukin-4 (IL-4) blockade improved tumor control. FIG. 5A shows two graphs describing immunocompetent C57BL/6 mice bearing Panc02 tumors that were left untreated or treated with 250 μg anti-OX40 and 250 μg anti-CTLA4 on day 14. On day 18 mice were randomized to no further treatment or twice weekly gemcitabine (100 mg/kg intraperitoneally) for 3 weeks and further randomized to receive no further treatment (panel (i)) or receive 100 μg anti-IL-4 intraperitoneally (i.p.) concurrent with gemcitabine injections (panel (ii)). Graphs show mean tumor area for each group with 6-7 mice per group. FIG. 5B shows a graph describing a tumor area on day 35 for groups receiving treatment combinations as in FIG. 5A. Each symbol represents one animal. Key: NS not significant; *p<0.05; **p<0.01; ***p<0.005; ****p<0.001).

FIGS. 6A-6C show improved efficacy with repeated cycles of immunochemotherapy. FIG. 6A is an analysis of peripheral blood immune cells following a cycle of chemoimmunotherapy showing six graphs describing representative gating for CD11b⁺ myeloid cells (panel (i)); Gr1^{HI} neutrophils in gated CD11b⁺ myeloid cells (panel (ii)); Gr1^{lo}MHCII⁺ monocytes in gated CD11b⁺ myeloid cells (panel (iii)); Ly6C⁺Ly6G⁻in gated CD11b⁺ myeloid cells (panel (iv)); CD8⁺ and CD4⁺T cells (panel (v)); and CD4⁺CD25⁺T cells (panel (vi)). FIG. 6B shows six scatter graphs providing a quantitative analysis of populations gated as in FIG. 6A in whole peripheral blood following one cycle of chemoimmunotherapy. Each symbol represents one mouse. FIG. 6C shows six graphs describing C57BL/6 mice bearing Panc02 tumors that were left untreated or treated with anti-OX40 (250 μg) and anti-CTLA4 (250 μg) on day 14. On day 18 mice were randomized to no further treatment or twice weekly gemcitabine (100 mg/kg intraperitoneally) for 2 weeks. Three (3) days following the last dose of gemcitabine select groups received another dose of anti-OX40 and anti-CTLA4 or no treatment followed by another cycle of twice weekly gemcitabine (100 mg/kg intraperitoneally) for 2 weeks. Graphs show tumor area for individual mice with 6-7 mice per group. Key: NS not significant; *p<0.05; **p<0.01; ***p<0.005; ****p<0.001).

FIG. 7 depicts three graphs showing alternate timing of chemotherapy. C57BL/6 mice bearing Panc02 tumors were left untreated or treated with 250 μg anti-OX40 and 250 μg anti-CTLA4 on day 11 (day 7) or on day 18 (day 0). Mice were randomized to no further treatment or twice weekly gemcitabine (GZ 100 mg/kg intraperitoneally) for 3 weeks starting day 18. Graphs show survival curves for mice with 6-7 mice per group for NT versus GZ alone (panel (i)); GZ alone versus anti-OX40 and anti-CTLA4 plus day 0 GZ (panel (ii)); and GZ alone versus anti-OX40 and anti-CTLA4 plus day 7 GZ (panel (iii)).

FIGS. 8A and 8B show improved efficacy of radiation with anti-CTLA4 pre-treatment of CT26 colorectal tumors. FIG. 8A provides graphs showing mean tumor size (panel (i)) and overall survival (panel (ii)). Mice were euthanized when tumors were greater than 12 mm in diameter or showed physical deterioration. FIG. 8B provides graphs depicting tumor measurements from individual mice in the following groups: untreated (panel (i)) or treated with anti-CTLA4 d7 (panel (ii)); radiotherapy (RT) 20Gy d14 (panel (iii)); anti-CTLA4 d7+RT 20Gy d14 (panel (iv)); anti-CTLA4 d15+RT 20Gy d14 (panel (v)); anti-CTLA4 d19+RT 20Gy d14 (panel (vi)). Representative experiment shown with n=6 mice per group. Experiment replicated a minimum of two times.

FIG. 9 is a graph showing the effect of anti-CTLA4 pre-treatment in 4T1 tumor bearing mice. 4T1 tumors are an animal model for stage 4 breast cancer. Tumor measurements from individual mice in groups untreated (panel (i)) or treated with anti-CTLA4 d7 (panel (ii)); radiotherapy (RT) 20Gy d14, 15, and 16 (panel (iii)); anti-CTLA4 d7+RT 20Gy d14, 15 and 16 (panel (iv)); anti-CTLA4 d17+RT 20Gy d14, 15 and 16 (panel (v)). Experiment replicated a minimum of two times.

FIG. 10 is a graph of overall survival in mice bearing CT26 colorectal tumors, showing optimum timing of anti-OX40 immunotherapy after radiation therapy. Mice bearing CT26 tumors in the right leg were left untreated or treated with 20Gy focal radiation. Mice were randomized to receive 250 μg anti-OX40 day 7, day 15 or day 19. Mice were euthanized when tumors were greater than 12 mm in diameter or when they showed physical deterioration. Data combined from 3 experiments, total n=12-18 mice per group.

FIGS. 11A-11C show that radiation efficacy was improved by pre-depletion of T regulatory cells. Mice bearing CT26 tumors in the right leg were randomized to receive no treatment, CD4 depleting antibodies or CD25 depleting antibodies on day 7. Mice were further randomized to be left untreated or treated with 20Gy focal radiation on day 14. FIG. 11A depicts cell sorting of peripheral blood lymphocytes gated to show CD8 and CD4 T cells in control (panel (i)) and CD4 depleted mice (panel (ii)), and CD4 T cells gated to show CD25⁺T cells in control (panel (iii)) and CD25 depleted mice (panel (iv). FIG. 11B provides graphs depicting tumor measurements from individual mice in given groups: untreated (panel (i)) or treated with anti-CD4 (panel (ii)); anti-CD25 (panel (iii)); radiotherapy (RT) (panel (iv)); anti-CD4+RT (panel (v)); anti-CD25+RT (panel (vi)). FIG. 11C is a graph showing overall survival. Mice were euthanized when tumors were greater than 12 mm in diameter or when they showed physical deterioration. Representative experiment shown with n=6 mice per group.

FIGS. 12A and 12B shows a comparison of different anti-CTLA4 clones. Mice bearing CT26 tumors in the right leg were left untreated or treated with 250 μg anti-CTLA4 clone 9D9 or 250 μg anti-CTLA4 clone UC10 on day 7. Mice were further randomized to be left untreated or treated with 20Gy focal radiation on day 14. FIG. 12A depicts graphs showing mean tumor size (panel (i)) and overall survival (panel (ii)). Mice were euthanized when tumors >12 mm in diameter or physical deterioration. FIG. 12B are graphs depicting tumor measurements from individual mice in the following groups: untreated (panel (i)) or treated with anti-CTLA4 (9D9) d7 (panel (ii)); anti-CTLA4 (UC10) d7 (panel (iii)); RT 20Gy d14 (panel (iv)); anti-CTLA4 (9D9) d7 +RT 20Gy d14 (panel (v)); anti-CTLA4 (UC10) d7 +RT 20Gy d14 (panel (vi)). Representative experiment shown with n=6 mice per group.

### DETAILED DESCRIPTION OF THE INVENTION

The disclosure herein presents methods that are useful for enhancing the efficacy of cancer chemotherapy.

The disclosure herein presents the discovery that combined administration of an agonistic anti-OX40 antibody and an anti-CTLA4 antibody to mice with established murine pancreatic adenocarcinoma tumors resulted in a transient phenotypic change associated with repolarization of macrophages in the tumor. Administration of gemcitabine concurrent with macrophage repolarization resulted in significantly improved tumor control compared to either chemotherapy or combined immunotherapy alone. The therapeutic window of this immunochemotherapy was short-lived. The return of the suppressive tumor environment was associated with Th2 polarization of CD4 T cells in the draining lymph node, increased CD4 infiltration of the tumor and rebounding M2 differentiation of tumor macrophages. Administration of IL-4 blocking antibodies improved tumor control by immunochemotherapy. Importantly, mice retained immune function following chemotherapy and additional cycles of immunochemotherapy were able to improve tumor control. These data demonstrate that, in a preclinical tumor model that is highly resistant to immunotherapy and chemotherapy, preparative immunotherapy can be used to improve tumor control to conventional chemotherapy.

Furthermore, it was discovered that radiation therapy delivered following immunotherapy with anti-CTLA4 resulted in 100% tumor cure in mice with established colorectal carcinoma tumors. Administration of anti-OX40 agonist antibody was optimal when delivered one day following radiation (Median survival not reached versus 50 days with RT alone, p<0.05). Anti-CTLA4 was highly effective when given prior to radiation, in part mediated by T regulatory cell depletion, while anti-OX40 agonist antibody was highly effective when delivered immediately following radiation, consistent with the timing of antigen release and increased antigen presentation. These data demonstrate that the combination of immunotherapy and radiation results in improved therapeutic efficacy; and that the ideal timing of administration with radiation is dependent on the type of immunotherapy utilized.

In further embodiments, the immunotherapy disclosed herein could be used for treatment of including, but not limited to breast cancer, pancreatic cancer, and lung cancer.

### Tumor Immune Environment

The immune environment of the tumor is predictive of outcome following conventional therapies. In mouse models of pancreatic cancer, therapies that decrease infiltrate of tumor-associated macrophages improved the response to chemotherapy. Similar results have also been observed in mouse mammary cancer models.

For those patients with an immune environment that promotes tumor growth it is proposed that there is an opportunity to improve the tumor environment through immunotherapy to improve outcome with conventional therapies. Immunotherapies targeting OX40 or CTLA4 have been shown to remodel the tumor environment via a change in T cell infiltrates. Immunotherapy with agonistic antibodies to OX40 was able to remodel tumors, resulting in increased CD8 infiltrate and as a consequence, decreased macrophage infiltrate (Gough et al., Cancer Res 2008; 68:5206-15). Similarly, it has been shown that blocking antibodies to CTLA-4 resulted in increased T cell infiltrate to tumors, both in mouse models (Curran et al., Proc. Natl. Acad. Sci. U.S.A. 2010; 107:4275-80) and in patients (Huang et al., Clinical cancer research 2011; 17:4101-9). However, the mere presence of these infiltrates in patients was not necessarily associated with therapeutic success (Huang et al., Clinical cancer research 2011; 17:4101-9). It has been shown that macrophages in the tumor immune environment could rapidly change their phenotype from pro-adaptive immune M1 differentiation to pro-wound healing M2 differentiation and resolve the initial inflammation following T cell therapy (Gough et al., Immunology 2012; 136:437-47). Nevertheless, the initial T cell infiltrate into tumors following systemic immunotherapy may be sufficient to transiently remodel the tumor environment, for example by restructuring or normalizing the inefficient neoangiogenic vasculature (Ganss et al., Cancer Res 2002; 62:1462-70), since the efficacy of chemotherapy is limited by inefficient drug delivery. Without being bound to a particular theory, it was hypothesized that tumor remodeling by immunotherapy has the potential to render tumors more susceptible to chemotherapy in other tumor immune environments.

To test this hypothesis, the Panc02 mouse model of pancreatic adenocarcinoma that forms a highly chemo- and radio-resistant tumor in immunocompetent mice was used, with extensive stromal involvement and diminished drug penetration compared to more immunogenic tumors. As demonstrated herein, systemic immunotherapy transiently changed the polarization of macrophages in tumors as determined by decreased arginase expression. Delivery of gemcitabine chemotherapy during the window of changed macrophage polarization resulted in significantly improved tumor control and survival. Additionally, it was demonstrated that T cell differentiation in these tumor-bearing mice was not optimal for this immunochemotherapy. This resulted in Type 2 helper T cell (Th2) differentiation associated with interleukin-4 (IL-4) production by activated CD4 T cells. Inhibiting interleukin-4 (IL-4) in vivo significantly improved the efficacy of immunochemotherapy. Finally, murine immune cells were shown to remain functional following chemotherapy such that additional rounds of immunochemotherapy significantly increased tumor control and survival. These data demonstrate that the sequence and timing of immunotherapy and chemotherapy can have a significant influence on the tumor microenvironment and tumor response. Preparative immunotherapy is a novel treatment option with the potential to improve the efficacy of chemotherapy where the immune environment is poor, and may increase response rates in cancers with negative immunology.

Radiation therapy influences the patient's immune system, and the immune system influences the response to radiation therapy (Gough et al., Immunotherapy, 2012. 4(2): 125-8). Radiation therapy of tumors results in a dose-responsive increase in MHC class I expression (Reits et al., The Journal of experimental medicine, 2006. 203(5): 1259-71) and a short window of antigen presentation within 2 days following high-dose radiation (Zhang et al., The Journal of experimental medicine, 2007. 204(1): 49-55). Many of the preclinical and clinical immune therapies targeting T cells thus apply costimulation or immune adjuvants closely following doses of radiation (Lee et al., Blood, 2009. 114(3): 589-595; Gough et al., J Immunother, 2010. 33(8): 798-809; Demaria et al., Clin Cancer Res, 2005. 11(2 Pt 1): 728-34; Deng et al., J Clin Invest, 2014. 124(2): 687-95; Seung et al., Sci Transl Med, 2012. 4(137): 137ra74). These approaches have been shown to varying degrees to increase tumor-antigen specific immune responses, improve clearance of radiation treated and distant untreated tumors, and protect cured animals from subsequent tumor challenge. However, a series of interesting anecdotal studies have demonstrated that immune therapy with Ipilimumab (human anti-CTLA4 antibody) followed by radiation can lead to extensive tumor regression in melanoma with increased tumor antigen specific responses (Postow et al., The New England journal of medicine, 2012. 366(10): 925-31; Hiniker et al., Translational Oncology, 2012. 5(6): 404-407). In these patients, radiation therapy was delivered in a palliative manner to individual lesions in patients already participating in Ipilimumab studies. Ipilimumab therapy has been shown to increase T cell infiltrates into tumors in patients, regardless of whether these tumors exhibit a response to antibody therapy (Huang et al., Clin Cancer Res, 2011. 17(12): 4101-9). Thus, those patients who achieved both local and distant disease control with focal palliative radiation delivered following immune therapy would likely have received treatment to an improved tumor environment. In a review of patients treated with Ipilimumab and radiation, Barker et al. found that patients treated with radiation following radiation therapy, in the ‘maintenance phase’, showed a significant survival advantage over those treated with radiation during the ‘induction phase’ (Barker et al., Cancer Immunol Res, 2013. 1(2): 92-8). These data indicate that the scheduling of anti-CTLA4 and radiation therapy can be improved by optimizing timing.

To date, few studies have rationally optimized the timing of immunotherapy with radiation such that immunotherapy is delivered first. It was recently demonstrated in preclinical murine models of radiation therapy that pre-treatment with TGFP inhibitors improved the response to radiation therapy by improving immune control of residual disease (Young et al., Cancer Immunol Res, 2014). Without being bound to a particular theory, it was hypothesized that pre-treatment with anti-CTLA4 antibodies before radiation therapy would improve tumor control compared to post-radiation treatment. In a preclinical model of colorectal cancer in immune competent mice, pre-treatment with anti-CTLA4 antibodies provided optimal tumor control. However, an alternate immunotherapy with anti-OX40, which targets recently-activated T cells, was optimal if delivered immediately following radiation therapy. Without being bound to a particular theory, the efficacy of anti-CTLA4 pretreatment may lay in its ability to delete T regulatory cells. The results described herein provide important preclinical evidence to consider when translating optimum combinatorial treatment to the clinic, specifically the immunotherapy mechanism of action may dictate the optimal timing with radiation.

### Anti-Tumor Therapy

Provided herein are methods for treating cancer, comprising administration of OX40 agonist or anti-OX40 antibody (e.g., an OX40 agonist antibody) and/or anti-CTLA4 antibody, in combination with other cancer treatments. Administration of an anti-OX40 antibody (e.g., an OX40 agonist antibody) and/or anti-CTLA4 antibody resulted in a change in the tumor environment (e.g., suppressed macrophage differentiation) and administration of this immunotherapy increased the anti-tumor effect of chemotherapy, e.g., varying levels of tumor regression, shrinkage, or a stalling in the advancement of the disease.

One aspect of the disclosure provides a method for treating cancer, comprising administering to a patient in need of treatment an effective amount of anti-OX40 antibody (e.g., an OX40 agonist antibody) and/or anti-CTLA4 antibody and one or more chemotherapeutic agents. Suitable chemotherapeutic agents and toxins are described in Remington's Pharmaceutical Sciences, 19th Ed. (Mack Publishing Co. 1995), and in Goodman and Gilman's the Pharmacological Basis of Therapeutics, 7th Ed. (MacMillan Publishing Co. 1985). Other suitable toxins and/or chemotherapeutic agents are known to those of skill in the art.

The administration of anti-OX40 antibody (e.g., an OX40 agonist antibody) and/or anti-CTLA4 antibody suppressed macrophage differentiation in tumors, as shown by a decrease in level of arginase expression in tumor associated macrophages. The suppression of tumor associated macrophage differentiation occurred in a window in which an anti-tumor effect by chemotherapy was observed in tumors otherwise resistant to conventional therapy. Accordingly, in certain embodiments of the invention, a chemotherapeutic agent (e.g., gemcitabine, SFU, docetaxel, paclitaxel, or CPT11) is administered at a time when macrophage differentiation is decreased. The administration of anti-OX40 antibody (e.g., an OX40 agonist antibody) and anti-CTLA4 antibody was also associated with Th2 differentiation of T cells that secrete IL4 which promotes macrophage differentiation. Administration of anti-IL4 antibody with the immunotherapy suppressed macrophage differentiation in response to IL4 secretion by the Th2 cells. Accordingly, in certain embodiments, use of anti-IL4 antibody is included in an anti-tumor regimen with anti-OX40 antibody (e.g., an OX40 agonist antibody) and anti-CTLA4.

Desirably, administration of an OX40 agonist and/or anti-CTLA4 antibody results in one or more of tumor remodeling, suppression of macrophage differentiation, and/or suppression of T cell differentiation. Thus, administration of the OX40 agonist and/or anti-CTLA4 antibody can be used to enhance the anti-tumor effect of conventional cancer therapy, including for example chemotherapy and radiotherapy. An OX40 agonist and/or an anti-CTLA4 antibody can be administered before, during or after chemotherapy or radiotherapy. An effective amount of an OX40 agonist and/or anti-CTLA4 antibody to be administered can be determined by a person of ordinary skill in the art by well-known methods. Where the toxicity of the cancer therapy is tolerated by the subject (e.g., having low lymphotoxicity), one or more rounds of immunochemotherapy according to the methods of the invention may be used.

Clinical response to administration of an OX40 agonist can be assessed using diagnostic techniques known to clinicians, including but not limited to magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, and chromatography. In one example, OX40 agonist and anti-CTLA4 antibody reduces macrophage differentiation, which can be measured by a decrease in arginase expression in macrophages (e.g., using the methods described herein).

Effective treatment with a cancer therapy including an OX40 agonist and/or anti-CTLA4 antibody includes, for example, reducing the rate of progression of the cancer, retardation or stabilization of tumor or metastatic growth, tumor shrinkage, and/or tumor regression, either at the site of a primary tumor, or in one or more metastases.

As reported herein below, administration of the OX40 agonist and the IDO inhibitor unexpectedly enhances the efficacy of the immunogenic composition comprising a tumor antigen.

### OX40 Agonists

OX40 agonists interact with the OX40 receptor on CD4+T-cells during, or shortly after, priming by an antigen resulting in an increased response of the CD4+T-cells to the antigen. An OX40 agonist interacting with the OX40 receptor on antigen specific CD4⁺T-cells can increase T cell proliferation as compared to the response to antigen alone. The elevated response to the antigen can be maintained for a period of time substantially longer than in the absence of an OX40 agonist. Thus, stimulation via an OX40 agonist enhances the antigen specific immune response by boosting T-cell recognition of antigens, e.g., tumor cells. OX40 agonists are described, for example, in U.S. Pat. Nos. 6,312,700, 7,504,101, 7,622,444, and 7,959,925, which are incorporated herein by reference in their entireties. Methods of using such agonists in cancer treatment are described, for example, in WO/2013/119202 and in WO/2013/130102 each of which are incorporated herein by reference in its entirety.

OX40 agonists include, but are not limited to OX40 binding molecules, e.g., binding polypeptides, e.g., OX40 ligand (“OX40L”) or an OX40-binding fragment, variant, or derivative thereof, such as soluble extracellular ligand domains and OX40L fusion proteins, and anti-OX40 antibodies (for example, monoclonal antibodies such as humanized monoclonal antibodies), or an antigen-binding fragment, variant or derivative thereof. Examples of anti-OX40 monoclonal antibodies are described, for example, in U.S. Pat. Nos. 5,821,332 and 6,156,878, the disclosures of which are incorporated herein by reference in their entireties. In certain embodiments, the anti-OX40 monoclonal antibody is 9B12, or an antigen-binding fragment, variant, or derivative thereof, as described in Weinberg, A. D., et al. J Immunother 29, 575-585 (2006), which is incorporated herein by reference in its entirety.

In certain aspects this disclosure provides a humanized anti-OX40 antibody or an antigen-binding fragment thereof comprising an antibody VH and an antibody VL, wherein the VL comprises an amino acid sequence at least 70%, 75%, 80%, 85%, 90%, 95%, or 100% identical to the reference amino acid sequence SEQ ID NO: 29 or SEQ ID NO: 32.

In certain aspects this disclosure provides a humanized anti-OX40 antibody or an antigen-binding fragment thereof comprising an antibody VH and an antibody VL, where the VL comprises SEQ ID NO: 29 or SEQ ID NO: 32.

The disclosure further provides a humanized anti-OX40 antibody or an antigen-binding fragment thereof comprising an antibody VH and an antibody VL, wherein the VH comprises VH-CDR1, VH-CDR2, and VH-CDR3 amino acid sequences identical to, or identical except for eight, seven, six, five, four, three, two, or one single amino acid substitutions, deletions, or insertions in one or more of the VH-CDRS to: the VHCDR1 amino acid sequence SEQ ID NO: 8, the VHCDR2 amino acid sequence SEQ ID NO: 14, SEQ ID NO: 15, or SEQ ID NO: 16, and the VHCDR3 amino acid sequence SEQ ID NO: 25, SEQ ID NO: 26, or SEQ ID NO: 27.

The disclosure further provides a humanized anti-OX40 antibody or an antigen-binding fragment thereof comprising an antibody VH and an antibody VL, wherein the VH comprises an amino acid sequence with the formula:

HFW1-HCDR1-HFW2-HCDR2-HFW3-HCDR3-HFW4,

wherein HFW1 is SEQ ID NO: 6 or SEQ ID NO: 7, HCDR1 is SEQ ID NO: 8, HFW2 is SEQ ID NO: 9, HCDR2 is SEQ ID NO: 14, SEQ ID NO: 15 or SEQ ID NO: 16, HFW3 is SEQ ID NO: 17, HCDR3 is SEQ ID NO: 25, SEQ ID NO: 26, or SEQ ID NO: 27, and HFW4 is SEQ ID NO: 28. In certain aspects the amino acid sequence of HFW2 is SEQ ID NO: 10, SEQ ID NO: 11, SEQ ID NO: 12, or SEQ ID NO: 13. In certain aspects the amino acid sequence of HFW3 is SEQ ID NO: 18, SEQ ID NO: 19, SEQ ID NO: 20, SEQ ID NO: 21, SEQ ID NO: 22, SEQ ID NO: 23, or SEQ ID NO: 24.

Moreover, the disclosure provides a humanized anti-OX40 antibody or an antigen-binding fragment thereof comprising an antibody VH and an antibody VL, wherein the VH comprises an amino acid sequence at least 70%, 75%, 80%, 85%, 90%, 95%, or 100% identical to the reference amino acid sequence SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67.

In one aspect, the disclosure provides a humanized anti-OX40 antibody or an antigen-binding fragment thereof comprising an antibody VH and an antibody VL, where the VL comprises the amino acid sequence SEQ ID NO: 29 and the VH comprises the amino acid sequence SEQ ID NO: 59.

In certain aspects the disclosure provides a humanized anti-OX40 antibody or an antigen-binding fragment thereof comprising an antibody heavy chain or fragment thereof and an antibody light chain or fragment thereof, where the heavy chain comprises the amino acid sequence SEQ ID NO: 71, and the light chain comprises the amino acid sequence SEQ ID NO: 30.

In other embodiments, the antibody which specifically binds to OX40, or an antigen-binding fragment thereof binds to the same OX40 epitope as mAb 9B12.

An exemplary humanized OX40 antibody is described by Morris et al., Mol Immunol. May 2007; 44(12): 3112-3121, and has the following sequence:

(SEQ ID NO: 95)
APLATDKTHTCPPCPAPELLGGPSVFLFPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSREEMTKNQVSLTCLVKGFYPSDIAVEWESNGQPENNY
KTTPPVLDSDGSFFLYSKLTVDKSRWQQGNVFSCSVMHEALHNH
YTQKSLSLSPGKELLGGGSIKQIEDKIEEILSKIYHIENEIARI
KKLIGERGHGGGSNSQVSHRYPRFQSIKVQFTEYKKEKGFILTS
QKEDEIMKVQNNSVIINCDGFYLISLKGYFSQEVNISLHYQKDE
EPLFQLKKVRSVNSLMVASLTYKDKVYLNVTTDNTSLDDFHVNG
GELILIHQNPGEFCVL

9B12 is a murine IgG1, anti-OX40 mAb directed against the extracellular domain of human OX40 (CD134) (Weinberg, A. D., et al. J Immunother 29, 575-585 (2006)). It was selected from a panel of anti-OX40 monoclonal antibodies because of its ability to elicit an agonist response for OX40 signaling, stability, and for its high level of production by the hybridoma. For use in clinical applications, 9B12 mAb is equilibrated with phosphate buffered saline, pH 7.0, and its concentration is adjusted to 5.0 mg/ml by diafiltration.

“OX40 ligand” (“OX40L”) (also variously termed tumor necrosis factor ligand superfamily member 4, gp34, TAX transcriptionally-activated glycoprotein-1, and CD252) is found largely on antigen presenting cells (APCs), and can be induced on activated B cells, dendritic cells (DCs), Langerhans cells, plamacytoid DCs, and macrophages (Croft, M., (2010) Ann Rev Immunol 28:57-78). Other cells, including activated T cells, NK cells, mast cells, endothelial cells, and smooth muscle cells can express OX40L in response to inflammatory cytokines (Id.). OX40L specifically binds to the OX40 receptor. The human protein is described in PCT Publication No. WO 95/21915. The mouse OX40L is described in U.S. Pat. No. 5,457,035. OX40L is expressed on the surface of cells and includes an intracellular, a transmembrane and an extracellular receptor-binding domain. A functionally active soluble form of OX40L can be produced by deleting the intracellular and transmembrane domains as described, e.g., in U.S. Pat. Nos. 5,457,035 and 6,312,700, and WO 95/21915, the disclosures of which are incorporated herein for all purposes. A functionally active form of OX40L is a form that retains the capacity to bind specifically to OX40, that is, that possesses an OX40 “receptor binding domain.”

In a related embodiment, the disclosure provides mutants of OX40L which have lost the ability to specifically bind to OX40, for example amino acids 51 to 183 of SEQ ID NO: 96, in which the phenylalanine at position 180 of the receptor-binding domain of human OX40L has been replaced with alanine (F180A).

>sp|P23510|TNFL4_HUMAN Tumor necrosis factor
ligand superfamily member 4 OS = Homo sapiens
GN = TNFSF4 PE = 1 SV = 1
(SEQ ID NO: 96)
MERVQPLEENVGNAARPRFERNELLLVASVIQGLGLLLCETYICL
HFSALQVSHRYPRIQSIKVQFTEYKKEKGFILISQKEDEIMKVQN
NSVIINCDGFYLISLKGYFSQEVNISLHYQKDEEPLFQLKKVRSV
NSLMVASLTYKDKVYLNVTTDNTSLDDFHVNGGELILIHQNPGEF
CVL

Methods of determining the ability of an OX40L molecule or derivative to bind specifically to OX40 are discussed below. Methods of making and using OX40L and its derivatives (such as derivatives that include an OX40 binding domain) are described in WO 95/21915, which also describes proteins comprising the soluble form of OX40L linked to other peptides, such as human immunoglobulin (“Ig”) Fc regions, that can be produced to facilitate purification of OX40 ligand from cultured cells, or to enhance the stability of the molecule after in vivo administration to a mammal (see also, U.S. Pat. No. 5,457,035 and PCT Publication No. WP 2006/121810, both of which are incorporated by reference herein in their entireties).

OX40 agonists include a fusion protein in which one or more domains of OX40L is covalently linked to one or more additional protein domains. Exemplary OX40L fusion proteins that can be used as OX40 agonists are described in U.S. Pat. No. 6,312,700, the disclosure of which is incorporated herein by reference in its entirety. In one embodiment, an OX40 agonist includes an OX40L fusion polypeptide that self-assembles into a multimeric (e.g., trimeric or hexameric) OX40L fusion protein. Such fusion proteins are described, e.g., in U.S. Patent No. 7,959,925, which is incorporated by reference herein in its entirety.

In certain embodiments, the OX40L fusion protein is a OX40L-IgG4-Fc polypeptide subunit or multimeric fusion protein. An OX40L fusion polypeptide subunit as described above can self-assemble into a trimeric or hexameric OX40L fusion protein. Accordingly, the disclosure provides a hexameric protein comprising six polypeptide subunits as described above. One exemplary polypeptide subunit self-assembles into a hexameric protein designated herein as “OX40L IgG4P Fusion Protein.” Except where specifically noted, the term “OX40L IgG4P Fusion Protein” as used herein refers to a human OX40L IgG4P Fusion Protein. The amino acid sequence of the polypeptide subunit that self-assembles into the hexameric protein OX40 IgG4P Fusion Protein is provided in SEQ ID NO: 98. Nonetheless, one of ordinary skill in the art will recognize that numerous other sequences also fulfill the criteria set forth herein for hexameric OX40L fusion proteins.

The disclosure further provides a polynucleotide comprising a nucleic acid that encodes an OX40L fusion polypeptide subunit, or a hexameric protein as provided herein, e.g., OX40L IgG4P Fusion Protein. An exemplary polynucleotide sequence that encodes a polypeptide subunit of OX40L IgG4P Fusion Protein is represented by SEQ ID NO: 97. In certain aspects, nucleic acid sequences encoding the IgG4 Fc domain, the trimerization domain and the OX40L receptor binding domains are joined in a 5′ to 3′ orientation, e.g., contiguously linked in a 5′ to 3′ orientation. In other aspects, the provided polynucleotide can further comprise a signal sequence encoding, e.g., a secretory signal peptide or membrane localization sequence. Polynucleotides encoding any and all OX40L fusion polypeptide subunits or multimeric, e.g., hexameric proteins comprising the subunits, are provided by this disclosure.

In certain aspects, the disclosure provides a polynucleotide comprising a nucleic acid that encodes OX40L IgG4P Fusion Protein. In certain aspects the nucleic acid sequence comprises SEQ ID NO: 97. Polynucleotides encoding control proteins provided herein, e.g., the disclosure provides a polynucleotide comprising a nucleic acid that encodes HuIgG-4FcPTF2OX40L F180A. In certain aspects the nucleic acid comprises SEQ ID NO: 99, and the expression product from this construct, also referred to herein as huIgGFcPTF2OX40L F180A comprises the amino acid sequence of SEQ ID NO: 100.

SEQ ID NO: 97: DNA Sequence of huIgG4FcPTF2OX40L
(5′ to 3′ Open Reading Frame)
GAGAGCAAGTACGGCCCTCCCTGCCCCCCTTGCCCTGCCCCCGAGTTC
CTGGGCGGACCTAGCGTGTTCCTGTTCCCCCCCAAGCCCAAGGACACC
CTGATGATCAGCAGAACCCCCGAGGTGACCTGCGTGGTGGTGGACGTG
TCCCAGGAGGACCCCGAGGTCCAGTTTAATTGGTACGTGGACGGCGTG
GAAGTGCATAACGCCAAGACCAAGCCCAGAGAGGAGCAGTTCAACAGC
ACCTACAGAGTGGTGTCCGTGCTGACCGTGCTGCACCAGGACTGGCTG
AACGGCAAGGAATACAAGTGCAAGGTCTCCAACAAGGGCCTGCCTAGC
AGCATCGAGAAGACCATCAGCAAGGCCAAGGGCCAGCCACGGGAGCCC
CAGGTCTACACCCTGCCACCTAGCCAAGAGGAGATGACCAAGAACCAG
GTGTCCCTGACCTGTCTGGTGAAAGGCTTCTATCCCAGCGATATCGCC
GTGGAGTGGGAGAGCAACGGCCAGCCCGAGAACAACTACAAGACCACC
CCCCCTGTGCTGGACAGCGACGGCAGCTTCTTCCTGTACTCCAGACTG
ACCGTGGACAAGTCCAGATGGCAGGAGGGCAACGTCTTCAGCTGCTCC
GTGATGCACGAGGCCCTGCACAACCACTACACCCAGAAGTCCCTGAGC
CTGAGCCTGGGCAAGGACCAGGATAAGATCGAGGCTCTGTCCTCCAAG
GTGCAGCAGCTGGAACGGTCCATCGGCCTGAAGGACCTGGCCATGGCT
GACCTGGAACAGAAAGTGCTGGAAATGGAAGCCTCCACACAGGTGTCA
CACAGATACCCCCGGATCCAGTCCATTAAGGTGCAGTTCACCGAGTAC
AAGAAAGAGAAGGGCTTTATCCTGACCTCCCAGAAAGAGGACGAGATC
ATGAAGGTGCAGAACAACTCCGTGATCATCAACTGCGACGGGTTCTAC
CTGATCTCCCTGAAGGGCTACTTCAGCCAGGAAGTGAACATCTCCCTG
CACTACCAGAAGGACGAGGAACCCCTGTTCCAGCTGAAGAAAGTGCGG
AGCGTGAACTCCCTGATGGTGGCCTCTCTGACCTACAAGGACAAGGTG
TACCTGAACGTGACCACCGACAACACCTCCCTGGACGACTTCCACGTG
AACGGCGGCGAGCTGATCCTGATCCACCAGAACCCTGGCGAGTTCTGC
GTGCTG
SEQ ID NO: 98: Amino Acid Sequence of
huIgG4FcPTF2OX40L (N to C terminus)
ESKYGPPCPPCPAPEFLGGPSVFLFPPKPKDTLMISRTPEVTCVVVDV
SQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRL
TVDKSRWQEGNVFSCSVMHEALHNHYTQKSLSLSLGKDQDKIEALSSK
VQQLERSIGLKDLAMADLEQKVLEMEASTQVSHRYPRIQSIKVQFTEY
KKEKGFILTSQKEDEIMKVQNNSVIINCDGFYLISLKGYFSQEVNISL
HYQKDEEPLFQLKKVRSVNSLMVASLTYKDKVYLNVTTDNTSLDDFHV
NGGELILIHQNPGEFCVL
DNA Sequence of huIgG4FcPTF2OX40L F180A
(5′ to 3′ Open Reading Frame)
(SEQ ID NO: 99)
GAGAGCAAGTACGGCCCTCCCTGCCCCCCTTGCCCTGCCCCCGAGTTC
CTGGGCGGACCTAGCGTGTTCCTGTTCCCCCCCAAGCCCAAGGACACC
CTGATGATCAGCAGAACCCCCGAGGTGACCTGCGTGGTGGTGGACGTG
TCCCAGGAGGACCCCGAGGTCCAGTTTAATTGGTACGTGGACGGCGTG
GAAGTGCATAACGCCAAGACCAAGCCCAGAGAGGAGCAGTTCAACAGC
ACCTACAGAGTGGTGTCCGTGCTGACCGTGCTGCACCAGGACTGGCTG
AACGGCAAGGAATACAAGTGCAAGGTCTCCAACAAGGGCCTGCCTAGC
AGCATCGAGAAGACCATCAGCAAGGCCAAGGGCCAGCCACGGGAGCCC
CAGGTCTACACCCTGCCACCTAGCCAAGAGGAGATGACCAAGAACCAG
GTGTCCCTGACCTGTCTGGTGAAAGGCTTCTATCCCAGCGATATCGCC
GTGGAGTGGGAGAGCAACGGCCAGCCCGAGAACAACTACAAGACCACC
CCCCCTGTGCTGGACAGCGACGGCAGCTTCTTCCTGTACTCCAGACTG
ACCGTGGACAAGTCCAGATGGCAGGAGGGCAACGTCTTCAGCTGCTCC
GTGATGCACGAGGCCCTGCACAACCACTACACCCAGAAGTCCCTGAGC
CTGAGCCTGGGCAAGGACCAGGATAAGATCGAGGCTCTGTCCTCCAAG
GTGCAGCAGCTGGAACGGTCCATCGGCCTGAAGGACCTGGCCATGGCT
GACCTGGAACAGAAAGTGCTGGAAATGGAAGCCTCCACACAGGTGTCA
CACAGATACCCCCGGATCCAGTCCATTAAGGTGCAGTTCACCGAGTAC
AAGAAAGAGAAGGGCTTTATCCTGACCTCCCAGAAAGAGGACGAGATC
ATGAAGGTGCAGAACAACTCCGTGATCATCAACTGCGACGGGTTCTAC
CTGATCTCCCTGAAGGGCTACTTCAGCCAGGAAGTGAACATCTCCCTG
CACTACCAGAAGGACGAGGAACCCCTGTTCCAGCTGAAGAAAGTGCGG
AGCGTGAACTCCCTGATGGTGGCCTCTCTGACCTACAAGGACAAGGTG
TACCTGAACGTGACCACCGACAACACCTCCCTGGACGACTTCCACGTG
AACGGCGGCGAGCTGATCCTGATCCACCAGAACCCTGGCGAGGCCTGC
GTGCTG
Amino Acid Sequence of huIgG4PFcTF2OX40L F180A
(N to C terminus)
(SEQ ID NO: 100)
ESKYGPPCPPCPAPEFLGGPSVFLFPPKPKDTLMISRTPEVTCVVVDV
SQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRL
TVDKSRWQEGNVFSCSVMHEALHNHYTQKSLSLSLGKDQDKIEALSSK
VQQLERSIGLKDLAMADLEQKVLEMEASTQVSHRYPRIQSIKVQFTEY
KKEKGFILTSQKEDEIMKVQNNSVIINCDGFYLISLKGYFSQEVNISL
HYQKDEEPLFQLKKVRSVNSLMVASLTYKDKVYLNVTTDNTSLDDFHV
NGGELILIHQNPGEACVL

The multimeric OX40L fusion protein exhibits increased efficacy in enhancing antigen specific immune response in a subject, particularly a human subject, due to its ability to spontaneously assemble into highly stable trimers and hexamers.

In another embodiment, an OX40 agonist capable of assembling into a multimeric form includes a fusion polypeptide comprising in an N-terminal to C-terminal direction: an immunoglobulin domain, wherein the immunoglobulin domain includes an Fc domain, a trimerization domain, wherein the trimerization domain includes a coiled coil trimerization domain, and a receptor binding domain, wherein the receptor binding domain is an OX40 receptor binding domain, e.g., an OX40L or an OX40-binding fragment, variant, or derivative thereof, where the fusion polypeptide can self-assemble into a trimeric fusion protein. In one aspect, an OX40 agonist capable of assembling into a multimeric form is capable of binding to the OX40 receptor and stimulating at least one OX40 mediated activity. In certain aspects, the OX40 agonist includes an extracellular domain of OX40 ligand.

The trimerization domain of an OX40 agonist capable of assembling into a multimeric form serves to promote self-assembly of individual OX40L fusion polypeptide molecules into a trimeric protein. Thus, an OX40L fusion polypeptide with a trimerization domain self-assembles into a trimeric OX40L fusion protein. In one aspect, the trimerization domain is an isoleucine zipper domain or other coiled coil polypeptide structure. Exemplary coiled coil trimerization domains include: TRAF2 (GENBANK® Accession No. Q12933, amino acids 299-348; Thrombospondin 1 (Accession No. P07996, amino acids 291-314; Matrilin-4 (Accession No. O95460, amino acids 594-618; CMP (matrilin-1) (Accession No. NP-002370, amino acids 463-496; HSF1 (Accession No. AAX42211, amino acids 165-191; and Cubilin (Accession No. NP-001072 , amino acids 104-138. In certain specific aspects, the trimerization domain includes a TRAF2 trimerization domain, a Matrilin-4 trimerization domain, or a combination thereof.

In particular embodiments, an OX40 agonist is modified to increase its serum half-life. For example, the serum half-life of an OX40 agonist can be increased by conjugation to a heterologous molecule such as serum albumin, an antibody Fc region, or PEG. In certain embodiments, OX40 agonists can be conjugated to other therapeutic agents or toxins to form immunoconjugates and/or fusion proteins.

In certain aspects, an OX40 agonist can be formulated so as to facilitate administration and promote stability of the active agent. In certain aspects, pharmaceutical compositions in accordance with the present disclosure comprise a pharmaceutically acceptable, non-toxic, sterile carrier such as physiological saline, non-toxic buffers, preservatives and the like. Suitable formulations for use in the treatment methods disclosed herein are described, e.g., in Remington's Pharmaceutical Sciences (Mack Publishing Co.) 16th ed. (1980).

### Anti-CTLA4 Antibodies

Antibodies that specifically bind CTLA4 and inhibit CTLA4 activity are useful for enhancing an anti-tumor immune response. Information regarding tremelimumab (or antigen-binding fragments thereof) for use in the methods provided herein can be found in U.S. Pat. No. 6,682,736 (where it is referred to as 11.2.1), the disclosure of which is incorporated herein by reference in its entirety. Tremelimumab (also known as CP-675,206, CP-675, CP-675206, and ticilimumab) is a human IgG2 monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression. Exemplary anti-CTLA4 antibodies are described for example at U.S. Pat. Nos. 6,682,736;

7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences are provided below (see U.S. Pat. No. 6,682,736.

Tremelimumab VH
(SEQ ID NO: 101)
GVVQPGRSLRLSCAASGFTFSSYGMHWVRQAPGKGLEWVAVIWYDGSN
KYYADSVKGRFTISRDNSKNTLYLQMNSLRAEDTAVYYCARDPRGATL
YYYYYGMDVWGQGTTVTVSSASTKGPSVFPLAPCSRSTSESTAALGCL
VKDYFPEPVTVSWNSGALTSGVH
Tremelimumab VL
(SEQ ID NO: 102)
PSSLSASVGDRVTITCRASQSINSYLDWYQQKPGKAPKLLIYAASSLQ
SGVPSRFSGSGSGTDFTLTISSLQPEDFATYYCQQYYSTPFTFGPGTK
VEIKRTVAAPSVFIFPPSDEQLKSGTASVVCLLNNFYPREAKV
Tremelimumab VH CDR1
(SEQ ID NO: 103)
GFTFSSYGMH
Tremelimumab VH CDR2
(SEQ ID NO: 104)
VIWYDGSNKYYADSV
Tremelimumab VH CDR3
(SEQ ID NO: 105)
DPRGATLYYYYYGMDV
Tremelimumab VL CDR1
(SEQ ID NO: 106)
RASQSINSYLD
Tremelimumab VL CDR2
(SEQ ID NO: 107)
AASSLQS
Tremelimumab VL CDR3
(SEQ ID NO: 108)
QQYYSTPFT

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequences shown herein above and a heavy chain variable region comprising the amino acid sequence shown herein above. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

Other anti-CTLA4 antibodies are described, for example, in US 20070243184. In one embodiment, the anti-CTLA4 antibody is Ipilimumab, also termed MDX-010; BMS-734016.

### Antibodies

Antibodies that selectively bind OX40, CTLA4, or IL4 and inhibit the binding or activity of OX40, CTLA4, and IL4, respectively, are useful in the methods of the invention. Subjects undergoing treatment involving immunotherapy may be administered virtually any anti-OX40, anti-CTLA4, or anti-IL4 antibody known in the art. Suitable antibodies include, for example, known antibodies, commercially available antibodies, or antibodies developed using methods well known in the art.

Antibodies useful in the invention include immunoglobulins, monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies disclosed herein), intrabodies, and epitope-binding fragments of any of the above. In particular, antibodies include immunoglobulin molecules and immunologically active fragments of immunoglobulin molecules, e.g., molecules that contain at least one antigen-binding site.

Antibodies of the invention encompass monoclonal human, humanized or chimeric antibodies. Antibodies used in compositions and methods of the invention can be naked antibodies, immunoconjugates or fusion proteins. In certain embodiments, the antibody is a human, humanized or chimeric antibody having an IgG isotype, particularly an IgG1, IgG2, IgG3, or IgG4 human isotype or any IgG1, IgG2, IgG3, or IgG4 allele found in the human population. Antibodies of the human IgG class have advantageous functional characteristics, such as a long half-life in serum and the ability to mediate various effector functions (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. The IgG1 subclass has the high ADCC activity and CDC activity in humans (Chemical Immunology, 65, 88 (1997)). In other embodiments, the antibody is an isotype switched variant of a known antibody.

### Pharmaceutical Compositions

The administration of a compound or a combination of compounds for the treatment of tumors or solid cancers may be by any suitable means that results in a concentration of the therapeutic that, combined with other components, has an anti-tumor effect or enhances the anti-tumor effect of chemotherapy (e.g., varying levels of tumor regression, shrinkage, or a stalling in the advancement of the disease). The compound may be contained in any appropriate amount in any suitable carrier substance. The composition may be provided in a dosage form that is suitable for parenteral (e.g., intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, N.Y.). Human dosage amounts can initially be determined by extrapolating from the amount of compound used in mice, as a skilled artisan recognizes it is routine in the art to modify the dosage for humans compared to animal models.

Compositions for parenteral use may be provided in unit dosage forms (e.g., in single-dose ampoules), or in vials containing several doses and in which a suitable preservative may be added (see below). Apart from the active agent(s), the composition may include suitable parenterally acceptable carriers and/or excipients. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Combination Therapies

In certain embodiments, the disclosure presented herein is a method of enhancing chemotherapy or radiotherapy efficacy in a subject having a colorectal cancer, the method comprising administering to the subject an anti-CTLA4 antibody and/or an OX40 agonist before, during or after chemotherapy or radiotherapy.

The potential interaction between immunotherapy and chemotherapy is being pursued by many investigators (Chen and Emens, Cancer immunology, immunotherapy: CII 2013; 62:203-16). Importantly, a prior study has demonstrated unexpectedly high response rates to chemotherapy following vaccine therapies in patients with non-small cell lung cancer (Antonia et al., Clinical cancer research 2006; 12:878-87). In this study, the vaccine alone was effective at generating antigen-specific T cell responses but did not affect disease progression in the majority of patients. However, vaccination therapies have not consistently synergized with chemotherapies to improve outcomes. Concurrent delivery of anti-CTLA4 with dacarbazine chemotherapy improved responses compared to darcarbazine alone in patients with metastatic melanoma (Robert et al., New England Journal of Medicine 2011; 364:2517-26), though response rates were consistent with that seen with anti-CTLA4 alone in previously-treated patients (Weber et al., Clinical cancer research 2009; 15:5591-8; Hodi et al., New England Journal of Medicine 2010; 363:711-23). In patients with non-small cell lung cancer given six rounds of paclitaxel and carboplatin, the addition of anti-CTLA4 concurrently with the first four doses of chemotherapy did not improve survival versus chemotherapy alone, though the addition of anti-CTLA4 concurrently with the last four doses of chemotherapy did improve progression free survival, though neither concurrent regimen affected overall survival (Lynch et al., Journal of clinical oncology; 30:2046-54). Similar results were seen in extensive disease small cell lung cancer patients where anti-CTLA4 concurrent with later doses of chemotherapy improved progression-free survival versus chemotherapy alone, but did not improve overall survival (Reck et al., Annals of oncology 2013; 24:75-83). Thus far no clinical studies have altered the timing of immunotherapy and chemotherapy to exploit the therapeutic window observed in the present preclinical studies.

Investigators have demonstrated that both chemotherapy and radiation therapy can render cancer cells more susceptible to immune destruction, through modulation of major histocompatibility complex (MHC) and costimulatory receptors (Reits et al., The Journal of experimental medicine 2006; 203:1259-71; Chakraborty et al., Cancer Res 2004; 64:4328-37; Ramakrishnan et al., The Journal of clinical investigation 2010; 120:1111-24). In addition, cell death caused by chemotherapy has been proposed to drive new tumor antigen-specific immune responses following treatment (Chen and Emens, Cancer immunology, immunotherapy : CII 2013; 62:203-16; Zitvogel et al., Nature reviews Immunology 2008; 8:59-73). Immunotherapy may also affect responses to chemotherapies via other mechanisms. The efficacy of chemotherapy is limited by drug penetration limiting the effective dose to cancer cells. Immunotherapy could improve the vascular organization of tumors by normalizing the neoangiogenic vasculature (Ganss et al., Cancer Res 2002; 62:1462-70), and interestingly, immunotherapy was also more effective through normalized vasculature (Hamzah et al., Nature 2008; 453:410-4). These data indicate that there may be a complex interplay between the immune status of the tumor and the response to therapy, and that via immunotherapy there is an opportunity to manipulate patient tumors to improve their sensitivity to chemotherapy.

Different systemic chemotherapies vary widely in their effect on systemic immune cells. There was increasing evidence that the FOLFIRINOX cocktail of chemotherapies provided an improvement in outcome in patients with metastatic pancreatic cancer, but like gemcitabine did not result in durable cures (Conroy et al., The New England journal of medicine 2011; 364:1817-25). However, this cocktail was significantly more lymphotoxic than gemcitabine. If one could boost the immune environment of the tumor using the array of immunotherapies that are moving towards clinical approval, the optimal chemotherapy partner might need reassessment with new criteria. Since it has now been shown in a wide variety of malignancies that the immune environment in the tumor significantly influences outcome to conventional therapies it is reasonable to hypothesize that improving the immune environment in the tumor via immunotherapy should improve outcomes to a range of conventional therapies. This may not greatly affect patients with excellent immune environments. For example across stages, colorectal carcinoma patients with good ‘immunoscores’ had excellent prognosis (Galon et al., Science 2006; 313:1960-4). However, for those with pro-tumor immune environments the prognosis was poor, regardless of stage (Galon et al., Science 2006; 313:1960-4). It is these patients who may benefit most from preparative immunotherapy. This approach may have greatest benefit in cancer types such as pancreatic adenocarcinoma, where tumors have very pro-tumor immune environments, are highly resistant to conventional therapies, and patient prognosis is poor.

The anti-tumor treatment defined herein may be applied as a sole therapy or may involve, in addition to the compounds of the invention, conventional surgery, bone marrow and peripheral stem cell transplantations, chemotherapy and/or radiotherapy.

### Kits

The invention provides kits for the treatment of tumors and solid cancers. In one embodiment, the kit includes an anti-OX40 antibody and an anti-CTLA4 antibody. In further embodiments, the kit contains a chemotherapeutic agent (e.g., gemcitabine). In additional embodiments, the kit contains an anti-IL4 antibody. In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. If desired an antibody of the invention (e.g., anti-OX40, anti-CTLA4, anti-IL4) is provided together with instructions for administering the antibody to a subject having a solid tumor.

In particular embodiments, the instructions include at least one of the following: description of the therapeutic agent; dosage schedule and administration for treatment of SCLC or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1:

### Immunotherapy Improved the Response to Chemotherapy.

To study whether immunotherapy could improve the response to chemotherapy, the Panc02 murine model of pancreatic adenocarcinoma was used. This model, like pancreatic adenocarcinoma in patients, is susceptible to cytotoxic agents at similar levels to other cell lines in vitro, but is highly resistant to chemotherapy and radiation therapy in vivo (Priebe et al., Cancer Chemother Pharmacol 1992; 29:485-9; Young et al., Cancer Immunol Res 2014). Panc02 tumors are highly infiltrated by macrophages in vivo, and it has been demonstrated that macrophage differentiation in Panc02 tumors is a significant factor limiting the in vivo efficacy of radiation therapy (Crittenden et al., PloS one 2012; 7:e39295).

To determine the effect of chemotherapy on macrophages in the tumor, mice bearing established Panc02 tumors were treated with gemcitabine chemotherapy and tumors were harvested after one week of treatment. Immunofluorescence histology demonstrated a broad macrophage infiltrate throughout the untreated tumor, particularly focused on the invasive margin, but also diffusely throughout the tumor (FIG. 1A). Following chemotherapy, macrophage infiltration was increased throughout the tumor (FIG. 1A), matching data from other murine pancreatic cancer cell lines (Mitchem et al., Cancer Res 2013; 73:1128-41) and murine mammary cancer models (DeNardo et al., Cancer discovery 2011; 1:54-67). To determine whether immunotherapy could modulate the differentiation of macrophages in Panc02 tumors, mice bearing established Panc02 tumors were treated with anti-OX40, anti-CTLA4, or anti-OX40 and anti-CTLA4 in combination. Tumor macrophages were isolated by flow cytometry at 4 or 7 days following immunotherapy (FIG. 1B), then analyzed by western blotting for arginase as a marker of suppressive/repair differentiation. The combination of antibodies decreased arginase expression in tumor macrophages at day 4, though this rebounded to elevated arginase expression by day 7 (FIG. 1C). Inducible nitric oxide synthase (iNOS) was not detected by western blotting in tumor macrophages under any treatment, suggesting there was not full conversion to a proinflammatory state. Interestingly, previous results with T cell targeted immunotherapy showed that active inflammatory resolution directed by tumor macrophages suppressed the transient benefits of T cell infiltration (Gough et al., Immunology 2012; 136:437-47). Without being bound to a particular theory, this indicates a finite window of immune-mediated remodeling in the tumor. Thus, a combination of anti-OX40 and anti-CTLA suppressed macrophage differentiation in the Panc02 tumor model.

### Example 2:

### Pretreatment with a Combination of Anti-OX40 and Anti-CTLA4 Significantly Improved Tumor Control with Chemotherapy.

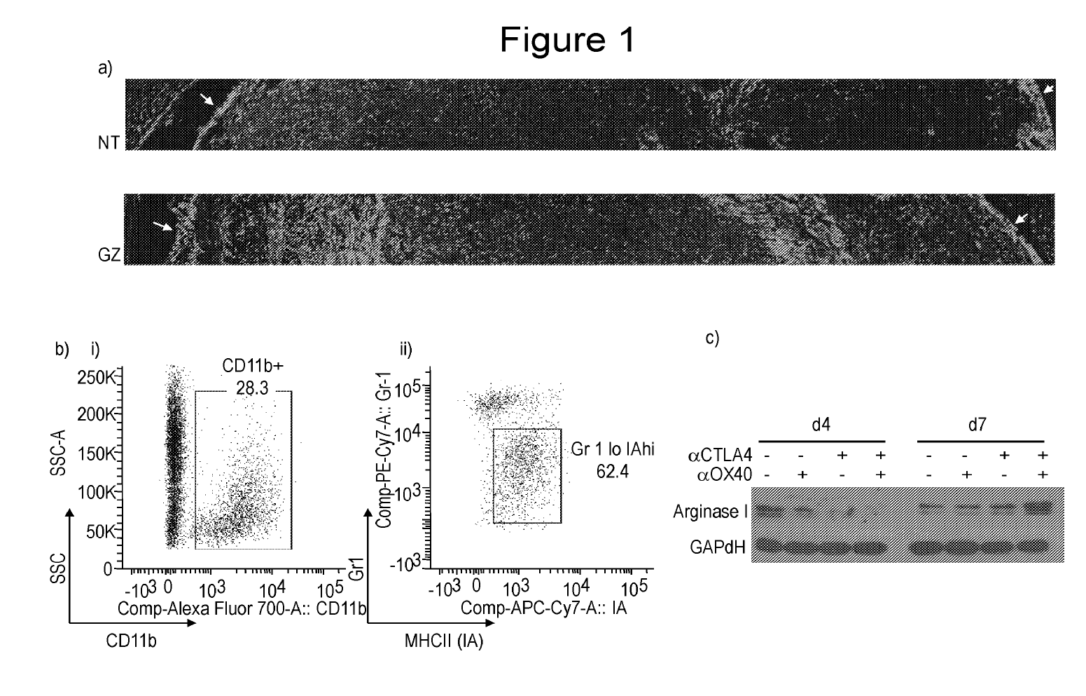

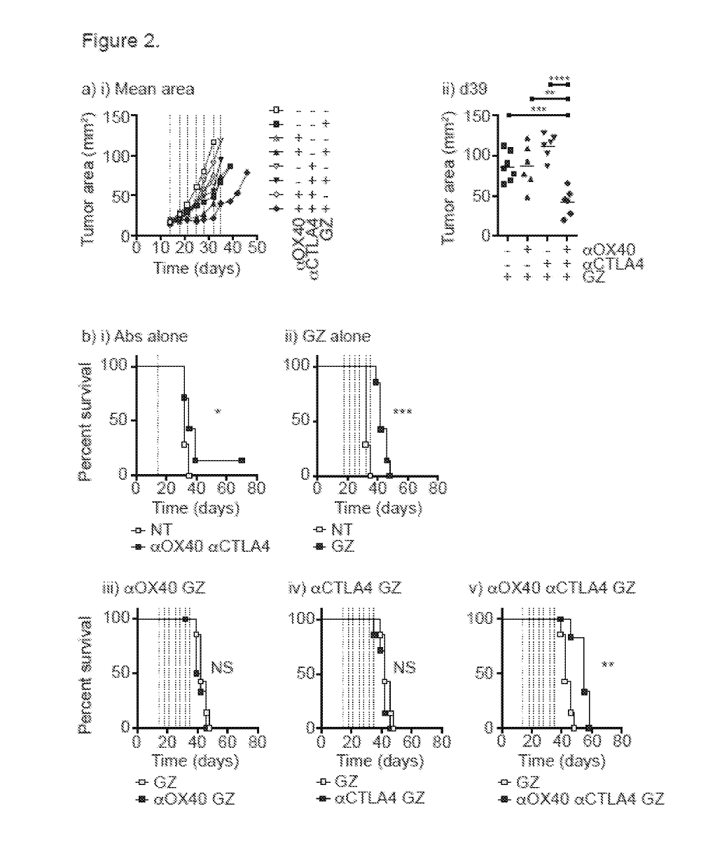

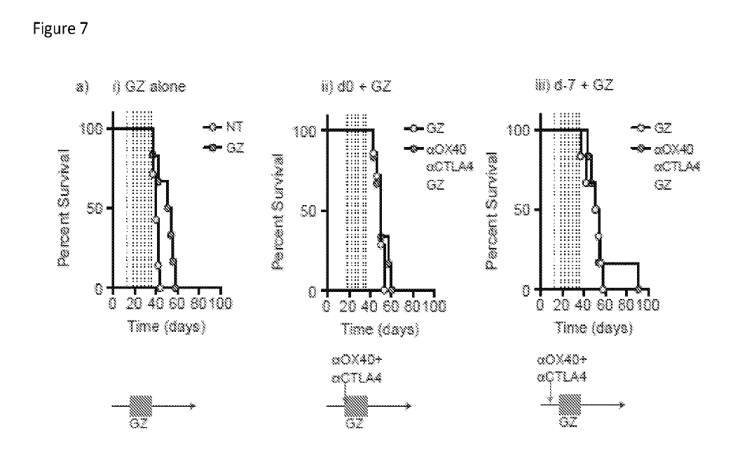

Based on this timing of macrophage differentiation, the effect of chemotherapy delivered starting 4 days following immunotherapy was tested. Immunotherapy alone was ineffective at tumor treatment in this model, while gemcitabine chemotherapy gave a transient tumor delay (FIG. 2A, panel (i)) and significantly extended survival (FIG. 2B, panel (ii)). Pre-treatment with anti-OX40 or anti-CTLA4 as single agents did not change the response to chemotherapy. However, pretreatment with the antibodies in combination with chemotherapy significantly improved tumor control with chemotherapy (FIG. 2A, panel (ii)) and improved survival compared to chemotherapy or the antibody combination alone (FIG. 2B). To determine how sensitive this effect is to timing, the effect of chemotherapy initiated on the same day as antibody immunotherapy, or 7 days following immunotherapy was tested. In each case survival was not different from chemotherapy alone (FIG. 7), indicating that this immunotherapy effect was sensitive to timing.

### Example 3:

### Effect of Immunotherapy on the Tumor Environment Over Different Time Points.

To examine the effect of immunotherapy on the tumor environment over these time points, tumors were harvested and flow cytometry for infiltrating cell populations was performed. Treatment combinations did not change the myeloid cell proportion, and surprisingly following treatment there was no statistically significant differences in the overall proportion of CD8 T cells in the tumor (FIG. 3). This poor infiltration of CD8 T cells in response to immunotherapy differs from the response to immunotherapy in more immunogenic tumor types (Gough et al., Cancer Res 2008; 68:5206-15; Redmond et al., Cancer Immunology Research 2013; 2:142-53), potentially explaining why the Panc02 tumor is poorly responsive to immunotherapy alone. Like CD8 T cells, CD11b⁺ myeloid cells did not change in proportion indicating that the changes in each cell population caused by immunotherapy were in differentiation rather than proportion. There was a significant increase in CD4 T cell infiltration 7 days following combined therapy (FIG. 3), and CD4 T cell infiltration has been shown to drive pro-tumor and immunosuppressive phenotypes in macrophages via IL-4 secretion. It has been demonstrated in other tumor models that anti-OX40 and anti-CTLA4 immunotherapy can synergize to drive CD4 T cells into a Type 2 helper T cells (Th2) differentiation pathway and direct IL-4 secretion (Redmond et al., Cancer Immunology Research 2013; 2:142-53; Linch et al., Oncoimmunology 2014; 3:e28245). These data would potentially explain the arginase rebound in tumor macrophages (FIG. 1C) because IL-4 is one of the dominant drivers of arginase expression in macrophages.

### Example 4:

### Immunotherapy Increased Type 2 Helper T Cell (Th2) Differentiation in the Panc02 Murine Model.

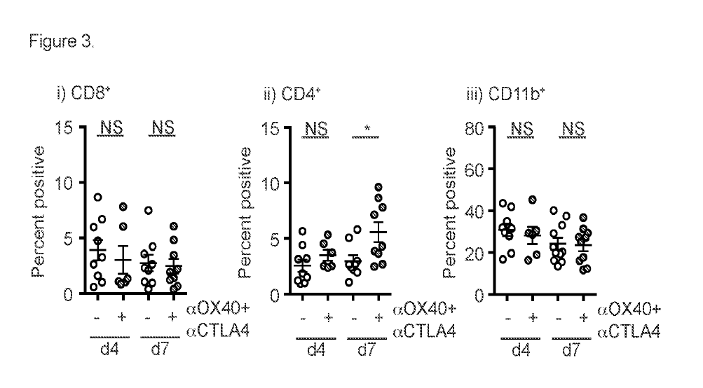

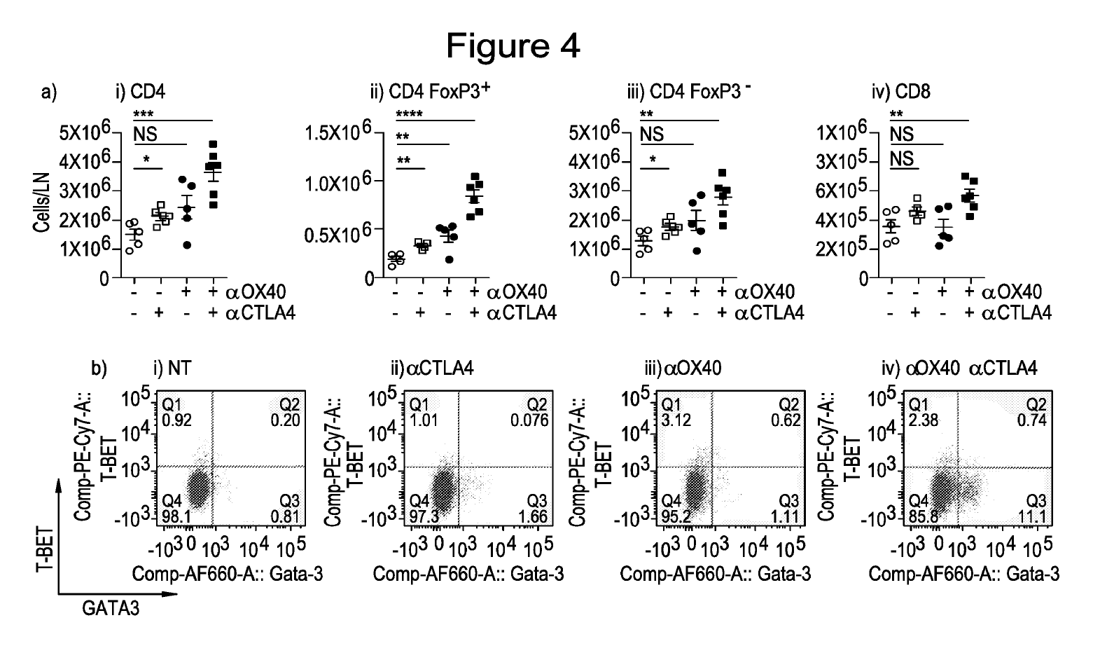

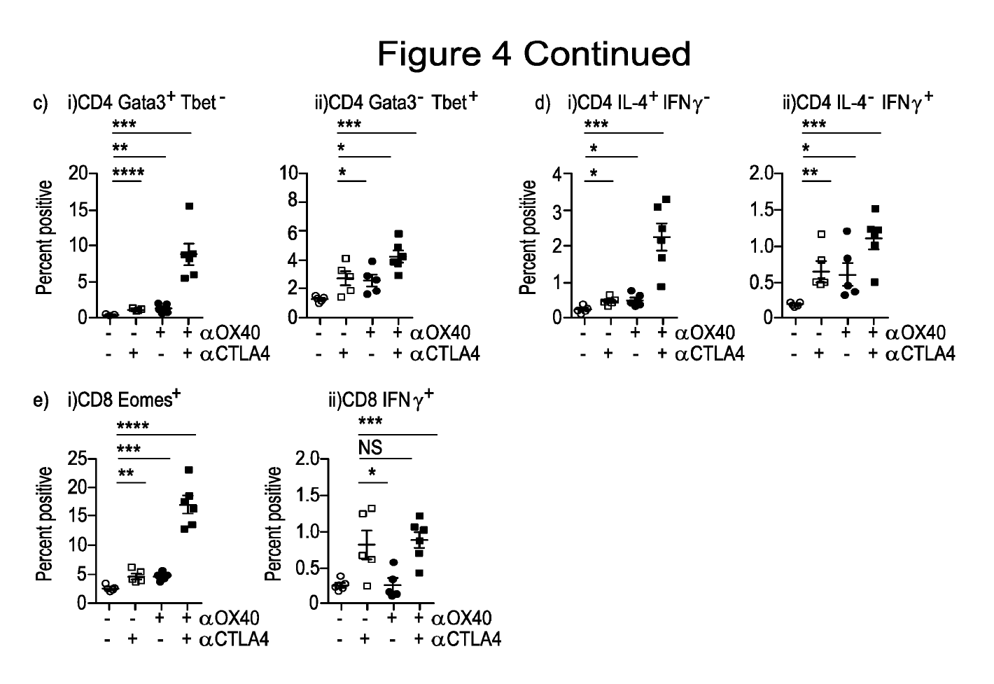

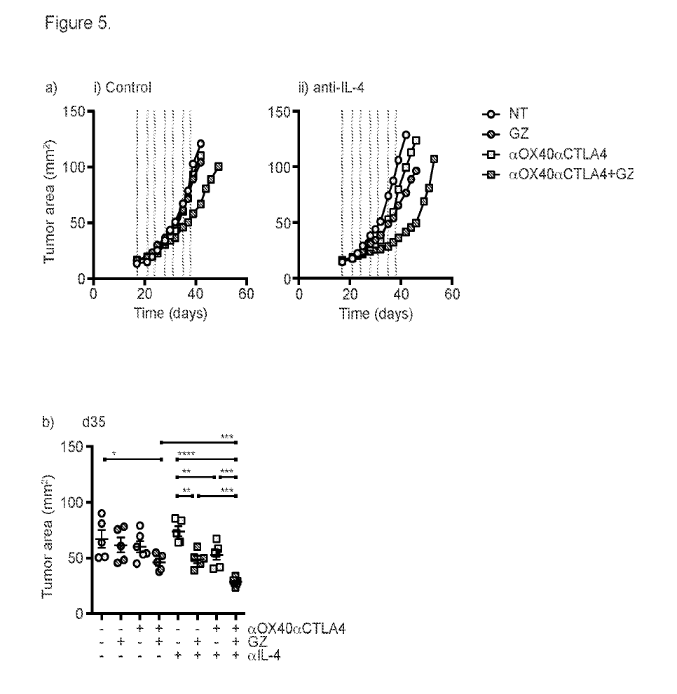

To determine whether immunotherapy was driving differentiation of Type 2 helper T cells (Th2) in this model, lymph nodes from Panc02 tumor-bearing mice treated with anti-OX40, anti-CTLA4 or the combination were isolated and T cell differentiation was analyzed. Combination treatment significantly increased CD4 and T regulatory cell numbers in lymph nodes, but only marginally increased CD8 T cell numbers (FIG. 4A). Transcription factor analysis of the non-regulatory (FoxP3⁻) CD4 T cells demonstrated synergy between anti-OX40 and anti-CTLA4 in induction of Gata3 expression (FIGS. 4B and 4C), which is indicative of Type 2 helper T cell (Th2) differentiation. The Type 1 helper T cell (Th1)-associated transcription factor Tbet was also upregulated, though to lower levels and appeared additive rather than synergistic in combination (FIG. 4C). To confirm these data, lymph node T cells from treated animals were stimulated in vitro with anti-CD3 and intracellular cytokine production was measured. Non-regulatory CD4 T cells from mice treated with anti-OX40 and anti-CTLA4 demonstrated synergistic induction of IL-4 production and additive induction of interferon gamma (IFNγ, FIG. 4D) closely matching the transcription factor data. Interestingly, in CD8 T cells combination therapy demonstrated significant upregulation of Eomes (Redmond et al., Cancer Immunology Research 2013; 2:142-53), indicating that the combination therapy is directing memory rather than effector T cell differentiation at this time.

### Example 5:

### Type 2 Helper T Cell (Th2) Production of IL-4 Limited the Effect of Chemotherapy in Combination with Treatment of Anti-OX40/Anti-CTLA4.

To determine whether this Type 2 helper T cell (Th2) production of IL-4 was limiting the effect of chemotherapy in this model, mice were treated with anti-OX40 and anti-CTLA4 and started on gemcitabine chemotherapy 4 days later. Matched groups of mice received IL-4 blocking antibodies at each administration of chemotherapy. Addition of anti-IL-4 did not affect tumor growth alone, but increased the impact of the chemotherapy and immunotherapy combination (FIG. 5A). The group given anti-OX40 and anti-CTLA4 pretreatment followed by chemotherapy delivered along with anti-IL-4 exhibited significantly improved tumor control at the end of the treatment period compared to all other groups (FIG. 5B). As shown above, on halting treatment with both chemotherapy and anti-IL-4 the tumor control persisted for approximately one week before the tumor resumed rapid growth.

### Example 6:

### The Adaptive Immune System was Sufficiently Functional Through Combination Treatment Plus Chemotherapy and Additional Combination Therapy Improved Survival.

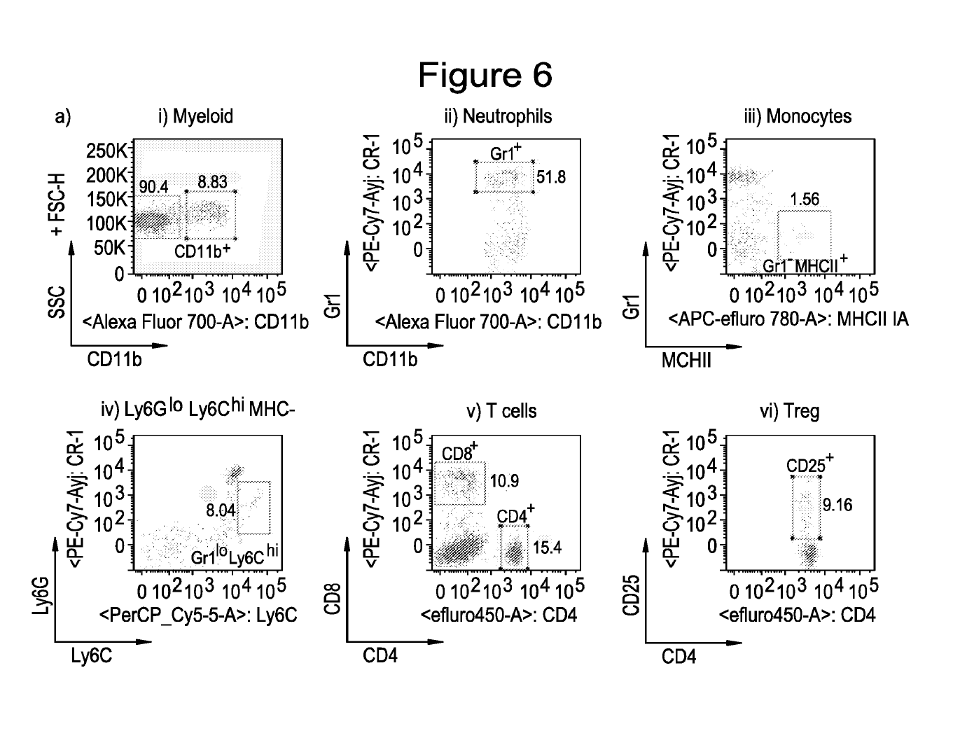

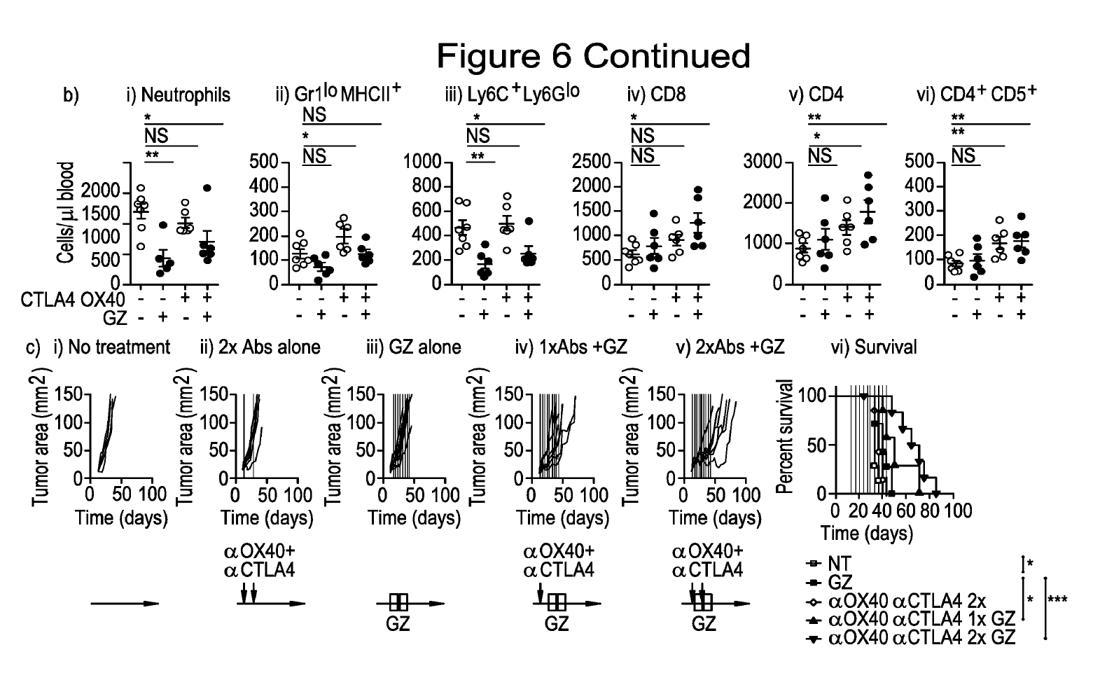

Different chemotherapies can have very different effects on hematopoietic cell populations. Gemcitabine is not one of the more myelotoxic or lymphotoxic chemotherapies, but it is possible that chemotherapy may limit the efficacy of immune therapies by killing effector populations. To determine the effect of treatment on immune cells, quantitative flow cytometry was performed on blood following immunochemotherapy. Using a range of phenotypic markers to identify sub-populations (FIG. 6A), it was demonstrated that gemcitabine significantly decreased CD11b⁺Gr1^{hi} neutrophils in the peripheral blood, as well as CD11b⁺Ly6C⁺Ly6G^{lo} immature myeloid cells (FIG. 6B). CD11b⁺Gr1⁻MHCII⁺ monocytes were increased by immunotherapy, and tended to decrease following chemotherapy but the change was not statistically significant. T cell populations were not decreased following chemotherapy, by contrast the numbers of CD8, CD4 and T regulatory cells were all increased in combination treatment plus chemotherapy compared to untreated control (FIG. 6B). These data indicate that the adaptive immune system remained intact in mice treated with gemcitabine chemotherapy. In this case, it was tested whether an additional round of immunotherapy could help to boost the response to chemotherapy. In this experiment mice were treated with combination immunotherapy followed 4 days later by chemotherapy, though for a shorter course of 2 weeks. The treatment course was shortened to ensure all mice were available for a second round of treatment. Mice were randomized to receive a second dose of combination immunotherapy followed 4 days later by a second 2-week round of chemotherapy. Mice receiving the second dose of immunotherapy exhibited significantly improved survival compared to mice receiving immunotherapy alone, chemotherapy alone or immunotherapy only one time (FIG. 6C). These data demonstrate that the adaptive immune system is sufficiently functional through chemotherapy to permit additional boosts that again enhance the efficacy of ongoing treatment.

These data demonstrate that preparative immunotherapy improved the response to chemotherapy and an improved response to chemotherapy coincided with a repolarization of tumor-associated macrophages. The window of opportunity was very narrow, and closure of the therapeutic window correlated with the emergence of Type 2 helper T cells (Th2) and upregulation of arginase I in tumor macrophages. Blocking the Type 2 helper T cell (Th2) effector cytokine IL-4 improved the efficacy of immunochemotherapy, and importantly, the immune system remained sufficiently functional through chemotherapy to permit at least one additional round of immunochemotherapy.

Pancreatic adenocarcinoma is known to have a highly suppressive immune environment and is also poorly responsive to chemotherapy in patients and in animal models. Some portion of this failure is believed to be due to very poor delivery of chemotherapy to cancer cells as a result of the highly fibrotic tumor environment and inefficient neoangiogenic vasculature. In certain tumor models, agonistic antibodies to OX40 or blocking antibodies to CTLA4 are sufficiently effective to remodel the tumor environment (Gough et al., Cancer Res 2008; 68:5206-15). However, in the model of pancreatic adenocarcinoma tested here, an effect on chemotherapy was only observed with combined therapy. In more immunogenic models where anti-CTLA4 alone is able to slow tumor growth, anti-CTLA4 was sufficient to improve the response to chemotherapy (Lesterhuis et al., PloS one 2013; 8:e61895; Jure-Kunkel et al., Cancer immunology, immunotherapy: CII 2013; 62:1533-45). In the poorly immunogenic Lewis lung carcinoma (3LL) tumor model, repeated administration of anti-CTLA4 with gemcitabine chemotherapy was able to generate a survival advantage where neither agent was effective alone (Lesterhuis et al., PloS one 2013; 8:e61895).

While different chemotherapy timings were tested following immunotherapy, altered schedules of immunotherapy were not tested. For example, tumor control has been demonstrated in other models by staggered doses of anti-OX40 and anti-CTLA4 immunotherapy (Redmond et al., Cancer Immunology Research 2013; 2:142-53). There remains a great deal of scope for optimization of the treatment plan with increasing the number of treatment cycles and addition of other agents such as anti-PD1, anti-41BB or other costimulatory molecules in development. Use of other agents could also be exploited to direct CD4 T cell differentiation away from the Type 2 helper T cell (Th2) pattern and IL-4 production to maximize tumor control.

### Example 7:

### Anti-CTLA4 Immunotherapy Prior to Radiotherapy Reduced Tumor Burden and Increased Overall Survival.

Increasingly, immunotherapy is being combined with radiation to enhance response. However, relatively little data exists regarding the ideal timing of combination therapy. Anecdotal reports demonstrate that palliative radiation delivered to patients undergoing anti-CTLA4 therapy resulted in systemic therapeutic responses (Postow et al., The New England journal of medicine, 2012. 366(10): 925-31; Hiniker et al., Translational Oncology, 2012. 5(6): 404-407). Given that these reports are incongruent with the majority of clinical trial designs which deliver anti-CTLA4 therapy concurrent with or following radiation, the effect of anti-CTLA4 immunotherapy timing with regards to radiation was investigated.

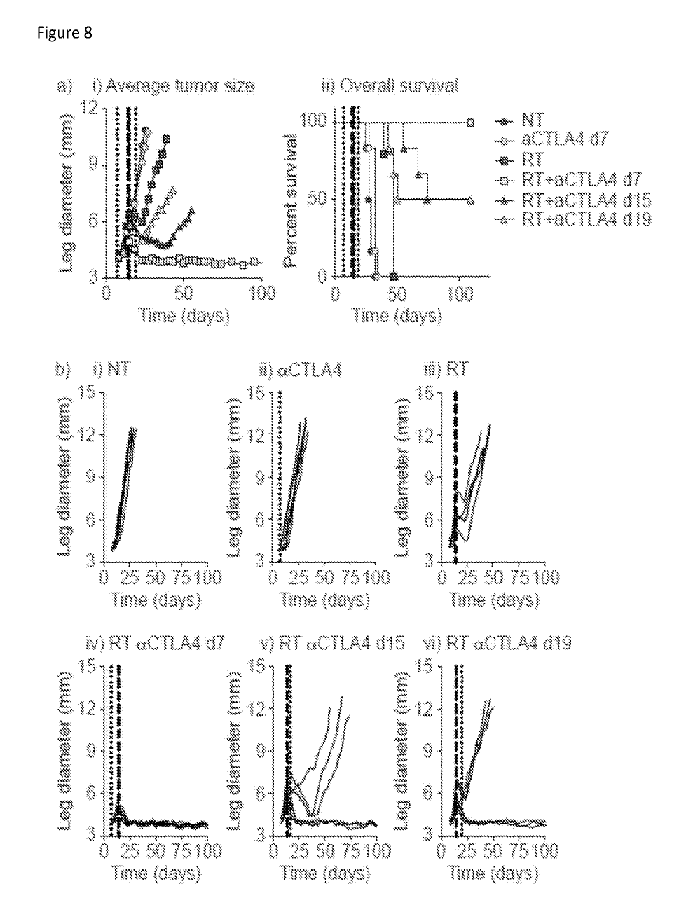

CT26 colorectal tumors were established in the right hindlimb of syngeneic BALB/c mice, and treated mice with anti-CTLA4 antibody on either day 7, day 15, or day 19; 20Gy radiation was delivered to the tumor only, on day 14. Anti-CTLA4 treatment alone on day 7 resulted in a small survival benefit with a median survival of 32 days versus 28 days in the no treatment (NT) control group (p=0.03) (FIGS. 8A and 8B, panels (i) and (ii)). While radiation alone resulted in transient tumor control, all tumors regrew resulting in euthanization secondary to tumor burden with a median survival of 47 days (p=0.0014 versus NT) (FIGS. 8A and 8B, panel (iii)). Tumor-bearing mice that received anti-CTLA4 on day 7 prior to radiation cleared their tumors with an undefined median survival (p=0.002 vs radiation alone) (FIGS. 8A and 8B, panel (iv)). The mean tumor size of mice pretreated with anti-CTLA4 versus control mice was not significantly different at the time of radiation therapy. Half the tumor-bearing mice that received anti-CTLA4 following radiation cleared the tumor with median survivals of 92 days for day 15 administration (p=0.002 vs radiation alone) versus 53 days for day 19 administration (p=0.07 vs radiation alone) (FIGS. 8A and 8B, panels (v) and (vi)). Importantly, all mice cured of tumors by combination therapy were resistant to rechallenge with CT26 tumors, but remained susceptible to a different tumor, indicating long-term antigen-specific immunity was achieved (Table 1, below).

TABLE 1
Tumor-bearing mice cured of CT26 tumors rejected rechallenge with
CT26, but succumbed to immunologically distinct 4T1 tumors.

Tumors from rechallenge with:

| CT26 primary tumor | CT26 | 4T1 |
|---|---|---|
| Anti-CTLA4 + RT | 0/17 | 17/17 |
| Anti-OX40 + RT | 0/13 | 13/13 |
| RT alone | 0/3 | 3/3 |

Tumor-bearing mice cured of CT26 tumors were rechallenged after 100 days with CT26 and 4T1 on opposing flanks. Resulting tumor growth demonstrated that all mice cured of CT26 rejected rechallenge with CT26, but succumbed to syngeneic, but immunologically distinct 4T1 tumors. These data demonstrate that the addition of anti-CTLA4 to radiation therapy improved survival at all timings, but was particularly effective when delivered before radiation.

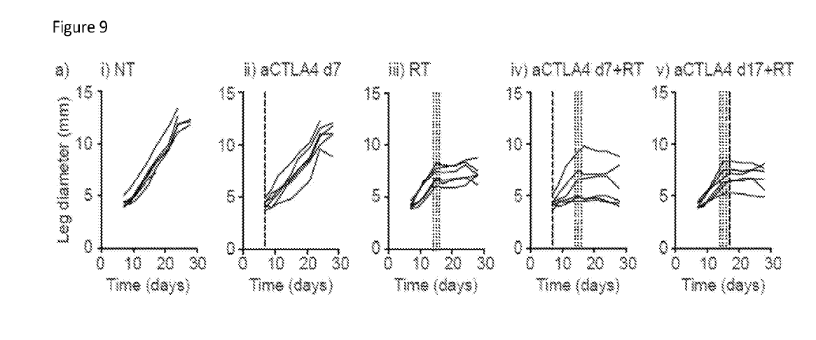

Prior reports demonstrated improved control of tumor growth where radiation was followed by anti-CTLA4 administration in a 4T1 mammary tumor model (Demaria et al., Clin Cancer Res, 2005. 11(2 Pt 1): 728-34; Dewan et al., Clinical cancer research: an official journal of the American Association for Cancer Research, 2009. 15(17): 5379-88). To determine whether the effect of timing was similar in this model, the timing of anti-CTLA4 administration with radiation was repeated in the 4T1 tumor model. BALB/c mice were challenged with 4T1 cells and given anti-CTLA4 on day 7 or day 17 with 20Gy of radiation delivered on days 14, 15, and 16, with 4T1 radiation dose and timing based on prior studies (Crittenden et al., PLoS One, 2013. 8(7): e69527). While mice were euthanized in all groups for worsening body condition secondary to lung metastases and therefore survival benefit of anti-CTLA4 therapy was unable to be determined, significantly smaller primary tumors were observed in mice that received anti-CTLA4 prior to radiation compared to radiation alone (p<0.05, FIG. 9, panels (i)-(v)). An improvement in tumor size was not detected with anti-CTLA4 given following radiation compared to radiation alone in this model (FIG. 9, panels (iii) and (v)). This post-radiation response was less effective than has previously been reported (Demaria et al., Clin Cancer Res, 2005. 11(2 Pt 1): 728-34; Dewan et al., Clinical cancer research : an official journal of the American Association for Cancer Research, 2009. 15(17): 5379-88), though to strictly test the effect of timing the study was restricted to a single administration of anti-CTLA4 rather than repeated administration as previously tested (Demaria et al., Clin Cancer Res, 2005. 11(2 Pt 1): 728-34; Dewan et al., Clinical cancer research : an official journal of the American Association for Cancer Research, 2009. 15(17): 5379-88). However, where survival is reported, even with repeat administration post-RT, anti-CTLA4 was shown to give no survival advantage in wild-type mice bearing 4T1 tumors compared to radiation alone (Pilones et al., Clin Cancer Res, 2009. 15(2): 597-606), consistent with the present data.

### Example 7:

### OX40 Immunotherapy After Radiotherapy Increased Overall Survival.

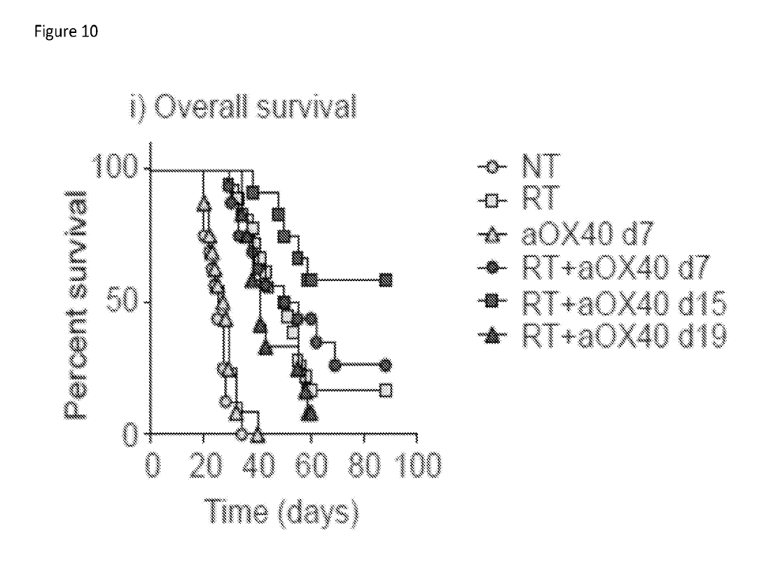

To determine whether the timing of anti-CTLA4 immunotherapy was uniquely based on anti-CTLA4′s mechanism of action, the ideal timing of anti-OX40 immunotherapy with radiation was evaluated. Anti-OX40 is induced on T cells immediately following antigen exposure (Evans et al., J Immunol, 2001. 167(12): 6804-11), and delivery of anti-OX40 following radiation therapy significantly increases survival in the 3LL lung carcinoma model (Gough et al., J Immunother, 2010. 33(8): 798-809; Yokouchi et al., Cancer Sci, 2008. 99(2): 361-7). CT26 colorectal tumors were established in the hindlimb of BALB/c mice and an anti-OX40 agonist antibody was delivered on day 7, day 15, or day 19; 20Gy radiation was delivered to the tumor only on day 14. Contrary to what was observed with anti-CTLA4 therapy in combination with radiation, pretreatment with anti-OX40 antibodies did not provide any therapeutic advantage over radiation alone (median survival 55 days versus 48 days, p=0.23) (FIG. 10). Much delayed anti-OX40 administration at day 19, also did not provide a benefit over radiation alone (median survival 41 days, p=0.6). However, anti-OX40 delivered one day following radiation resulted in ˜50% tumor clearance (116.5 days, p=0.0006 vs radiation alone) (FIG. 10). This timing agrees with prior studies demonstrating that anti-OX40 must be present during the key period 12-24 hours following antigen exposure to coincide with OX40 upregulation on T cells (Evans et al., J Immunol, 2001. 167(12): 6804-11), and with the evidence of tumor antigen-presentation approximately 2 days following radiation therapy (Zhang et al.,. The Journal of experimental medicine, 2007. 204(1): 49-55), suggesting that 5 days post-radiation therapy is beyond this therapeutic window. Importantly, all mice cured of tumors by optimal timing were resistant to rechallenge with CT26 tumors, but remained susceptible to a syngeneic antigenically distinct tumor, indicating long term antigen-specific immunity was achieved (Table 1).

### Example 7:

### Improved Radiation Efficacy of Anti-CTLA4 Prior to Radiation is Based in Part on T Regulatory Cell Depletion.

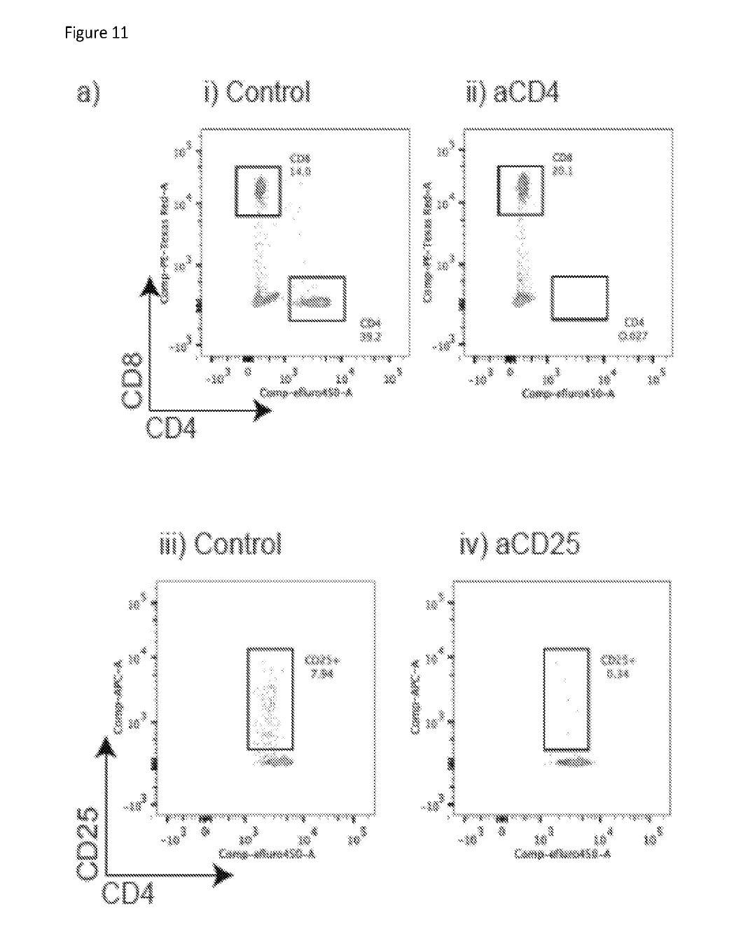

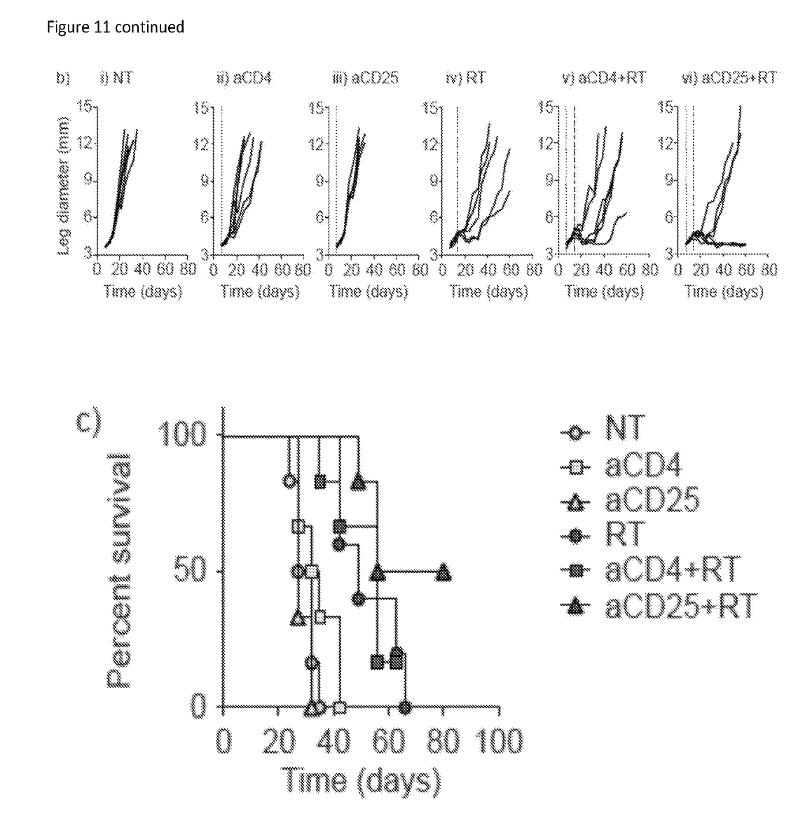

Recent reports demonstrate that anti-CTLA4 antibodies cause Fc-dependent depletion of T regulatory cells in the tumor (Simpson et al., J Exp Med, 2013. 210(9): 1695-710), and it has been shown that depletion of T regulatory cells concurrent or following radiation therapy resulted in enhanced tumor control (Bos et al., J Exp Med, 2013. 210(11): 2435-66; Sharabi et al., Cancer Immunol Res, 2014). To determine whether the improved radiation efficacy of anti-CTLA4 prior to radiation could be explained by T regulatory cell depletion, CT26 tumors were established in the hindlimb of BALB/c mice and treated on day 7 with anti-CD4 to deplete all CD4 T cells or anti-CD25 to deplete T regulatory cells. Mice were treated with radiation therapy on day 14 as above. Antibody treatment efficiently depleted CD4⁺ and CD25⁺ cells in the mouse (FIG. 11A). CD4 depletion did not affect tumor growth alone or in combination with subsequent radiation therapy (FIG. 11B). CD25 depletion did not affect tumor growth alone, but when followed by radiation therapy resulted in cure of tumors in half of the mice (FIG. 11C). Importantly, CD25 depletion did not perform as well as in prior studies with anti-CTLA4 pre-treatment (see FIGS. 8A and 8B), and total CD4 depletion, which would include T regulatory cell depletion, was not effective. Without being bound to a particular theory, this indicates that anti-CTLA4 provides effects in addition to T regulatory cell depletion, and that non-regulatory CD4 cells is important for the cures in CD25-depleted animals. However, it has been previously demonstrated that increased proportions of antigen-responsive CD8⁺CD25⁺ cells repopulate tumors following radiation therapy (Gough et al., J Immunother, 2010. 33(8): 798-809), and these cells would also be depleted by anti-CD25 treatment. Without being bound to a particular theory, it is likely that anti-CTLA4 therapy plays a dual role by both removing pre-existing T regulatory cells and the conventional effect of blocking CTLA4-mediated suppression of CD4 and CD8 effector T cells, permitting improved clearance of residual cancer cells following radiation therapy.

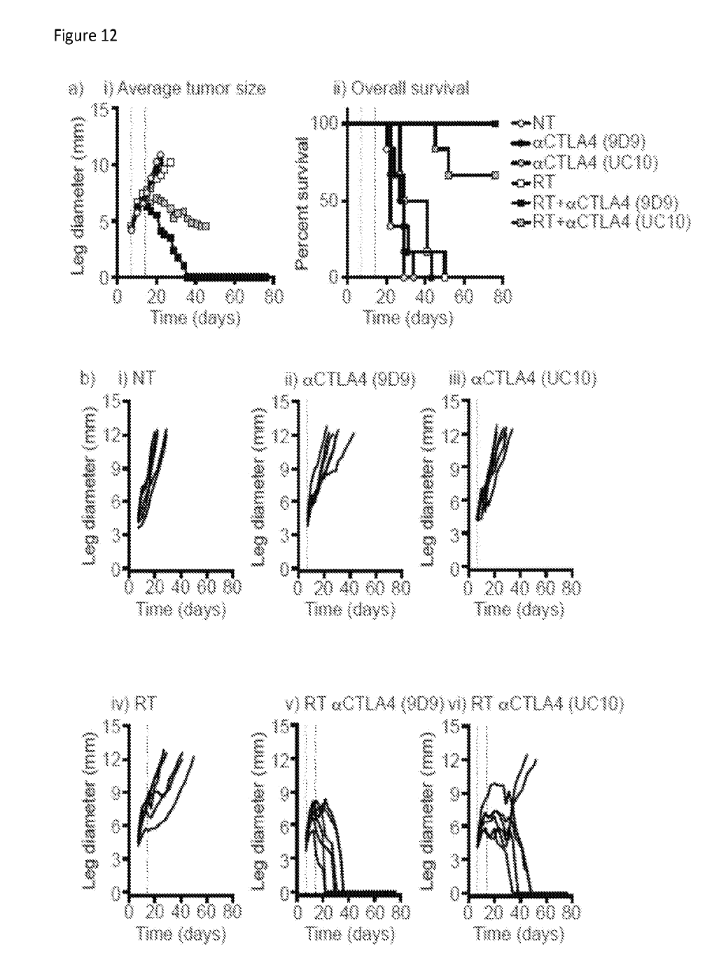

Since different anti-CTLA4 clones have been shown to differ in depletion of regulatory T cells, different clones were tested in combination with radiation therapy: the 9D9 clone which is highly depleting, and the UC10 clone which is less depleting (Simpsonet al., J Exp Med, 2013. 210(9): 1695-710). As before, CT26 tumors were established in the hindlimb of immunocompetent Balb/c mice and administered either the 9D9 clone or the UC10 clone on day 7 followed by radiation on day 14. While all mice treated with 9D9 and radiation cleared their tumors, 67% of mice treated with the UC10 clone cleared their tumors (FIG. 12). Taken together, these data indicate that the T regulatory cell depletion enhances tumor clearance, but is not exclusively responsible for the synergy seen between anti-CTLA pretreatment and radiation.

In this study, the ideal timing of anti-CTLA4 blockade or anti-OX40 agonist therapy in combination with radiation, which vary in accordance with their variable mechanisms of action. It was found that tumor preconditioning with anti-CTLA4 blockade followed by radiation resulted in clearance of murine colorectal tumors. These results are consistent with anecdotal case reports from patients with metastatic melanoma receiving Ipilimumab therapy who subsequently receive palliative radiation and have systemic abscopal responses with long-term disease free survival (Postow et al., The New England journal of medicine, 2012. 366(10): 925-31; Hiniker et al., Translational Oncology, 2012. 5(6): 404-407). Further, a retrospective review of patients receiving ipilumimab who underwent palliative radiation had improved overall survival if radiation was delivered during maintenance versus induction ipilumimab further demonstrating that preconditioning improved outcome (Barker et al., Cancer Immunol Res, 2013. 1(2): 92-8). In murine models, concurrent and post-RT treatment with anti-CTLA4 has been shown to control tumor growth (Demaria et al., Clin Cancer Res, 2005. 11(2 Pt 1): 728-34; Dewan et al., Clinical cancer research : an official journal of the American Association for Cancer Research, 2009. 15(17): 5379-88), but limited influence on overall survival, ranging from 0% (Pilones et al., Clin Cancer Res, 2009. 15(2): 597-606) to 20% (Belcaid et al., PLoS One, 2014. 9(7): e101764) overall survival with the combination of anti-CTLA4 and RT. The mechanism of action of anti-CTLA4 has been associated with its ability to deplete T regulatory cells in the tumor (Simpson, T. R., F. Li, W. Montalvo-Ortiz, M. A. Sepulveda, K. Bergerhoff, F. Arce, C. Roddie, J. Y. Henry, H. Yagita, J. D. Wolchok, K. S. Peggs, J. V. Ravetch, J. P. Allison, and S. A. Quezada, Fc-dependent depletion of tumor-infiltrating regulatory T cells co-defines the efficacy of anti-CTLA-4 therapy against melanoma. J Exp Med, 2013. 210(9): 1695-710), and depletion of T regulatory cells concurrent or post-RT has been shown to improve tumor control by radiation therapy (Bos et al., J Exp Med, 2013. 210(11): 2435-66; Sharabi et al., Cancer Immunol Res, 2014). The results described herein demonstrate that radiation followed by anti-CTLA4 blockade did improve radiation efficacy, but not to the same degree as pretreatment and that pretreatment depletion of T regulatory cells could also improve responses to radiation. These results are important given that the majority of ongoing clinical trials combining Ipilimumab and radiation deliver Ipilimumab concurrently and/or following radiation, which may result in improved outcomes, but may not be fully maximizing the potential for synergy.

Just as many chemotherapeutic agents work via unique mechanisms, immunotherapeutic agents have differing mechanisms of action. Whether different classes of immunotherapeutic agents may result in different ideal timing was investigated. It was found that anti-OX40 agonist antibodies, which act as T cell co-stimulatory agents, improved radiation efficacy when delivered shortly after radiation. The improved efficacy of combination therapy is consistent with the window of antigen presentation following hypofractionated radiation (Zhang et al., The Journal of experimental medicine, 2007. 204(1): 49-55). The OX40 molecule is upregulated on T cells rapidly and for a limited time following antigen engagement, and agonist antibodies must be present during that window for effective T cell stimulation (Evans et al., J Immunol, 2001. 167(12): 6804-11). While OX40 is expressed on T regulatory cells, administration of anti-OX40 to tumor-bearing mice does not result in depletion of tumor T regulatory cells (Gough et al., Cancer Res, 2008. 68(13): 5206-15). Anti-OX40 antibodies have recently shown promise in a phase I clinical trial (Curti et al., Cancer Res, 2013. 73(24): 7189-98), and are currently being evaluated in a Phase I trial in combination with radiation that uses the optimal timing.

In conclusion, it was discovered that the timing of immunotherapy in combination with radiation affects outcome. The ideal timing of specific immunotherapeutic agents is consistent with their mechanisms of action, and preclinical data regarding mechanism should be considered when combining agents and translating to the clinic.

The results described herein above were carried out using the following materials and methods.

### Animals and Cell Lines

The Panc02 murine pancreatic adenocarcinoma cell line (Priebe et al., 1992, Cancer Chemother Pharmacol; 29:485-9. C57BL/6) was kindly provided by Dr. Woo (Mount Sinai School of Medicine, N.Y.). 6-8 week old C57BL/6 mice were obtained from Charles River Laboratories (Wilmington, Mass.) for use in these experiments. All animal protocols were approved by the EACRI IACUC (Animal Welfare Assurance No. A3913-01).

The CT26 murine colorectal carcinoma (Brattain et al., Cancer Res, 1980. 40(7): 2142-6) and the 4T1 mammary carcinoma cell lines (Aslakson. and Miller, Cancer Research, 1992. 52(6): 1399-405) were obtained from ATCC (Manassas, Va.). Cells were grown in RPMI-1640 media supplemented with HEPES, non-essential amino acids, sodium pyruvate, glutamine, 10% FBS, penicillin and streptomycin. All cell lines tested negative for mycoplasma. BALB/c were obtained from Jackson Laboratories (Bar Harbor, Me.). All animal protocols were approved by the Earle A. Chiles Research Institute IACUC (Animal Welfare Assurance No. A3913-01).

### Immunochemotherapy

Mice bearing 10-14 day old tumors were treated with anti-OX40 (OX86, 250 μg intraperitoneally, BioXcell, West Lebanon, N.H.), anti-CTLA4 (9D9, 250 μg intraperitoneally, BioXcell) or the combination. Chemotherapy consisted of 100 mg/kg Gemcitabine (Eli Lilly and Co., Indianapolis, Ind.) intraperitoneally twice per week for 2 or 3 weeks. Anti-interleukin-4 (Anti-IL-4, 11B11, 100 μg intraperitoneally, BioXcell) was delivered intraperitoneally twice per week for 3 weeks.

### Antibodies and Reagents

Fluorescently-conjugated antibodies CD11b-AF700, Gr1-PE-Cy7, IA (major histocompatibility complex (MHC) class II)-e780, Ly6G-PE-Cy7, Ly6C-PerCP-Cy5.5, CD4-e450, CD4-PerCP Cy5.5, FoxP3-e450, CD25-APC, and CD8-FITC were obtained from eBioscience (San Diego, Calif.). CD4-v500, and Ly6G-FITC were obtained from BD Biosciences (San Jose, Calif.). CD8-PE-TxRD was obtained from Invitrogen (Carlsbad, Calif.). Rat anti-F4/80 was obtained from AbD Serotec (Raleigh, N.C.). Western blotting antibodies used included Arginase I (BD Biosciences), GAPdH, anti-mouse- horseradish peroxidase (HRP), and anti-rabbit-HRP (all Cell Signaling Technology, Danvers, Mass.).

Fluorescently-conjugated antibodies CD4-e450, CD25-APC, CD4-PerCP were obtained from eBioscience (San Diego, Calif.). CD8-PE-TxRD was obtained from Invitrogen (Carlsbad, Calif.). Therapeutic anti-CTLA4 (clone 9D9 or UC10), anti-OX40 (clone OX86), anti-CD4 (clone GK1.5), and anti-CD25 (clone PC.61.5.3) antibodies were obtained from BioXcell (Branford, Conn.) and resuspended in sterile PBS to a concentration of 1 mg/mL.

### In Vivo Radiation Therapy Models

1×10⁴ CT26 or 5×10⁴ 4T1 cells were injected in 100 μL of PBS subcutaneously in the right hind limb of immunocompetent BALB/c mice. Antibodies were administered as 250 μg (anti-OX40 and anti-CTLA4) or 100 μg (anti-CD4 and anti-CD25) intraperitoneally. Antibody therapy was administered at designated timepoints indicated in each procedure. Radiation was delivered using the clinical linear accelerator (6MV photons, Elekta Synergy linear accelerator, Atlanta, Ga.) with a half-beam block to protect vital organs and 1.0 cm bolus to increase the dose to the tumor. For CT26 tumors, 20Gy×1 was delivered on day 14 (Young et al., Cancer Immunol Res, 2014); for 4T1 tumors 20Gy×3 was delivered on days 14 though 16 (Crittenden et al., PLoS One, 2013. 8(7): e69527). For mice cured of CT26 tumors, mice were rechallenged with 5×10⁴ 4T1 and 1×10⁴ CT26 tumors in opposite flanks to assess tumor-specific immunity.

### Immunohistology

For immunohistology, tumors were fixed overnight in Z7 zinc based fixative (Lykidis et al., 2007, Nucleic acids research; 35:e85). Tissue was then dehydrated through graded alcohol to xylene, incubated in molten paraffin, and then buried in paraffin. Sections (5 μm) were cut and mounted for analysis. Tissue sections were boiled in ethylenediaminetetraacetic acid (EDTA) buffer as appropriate for antigen retrieval. Primary antibody binding was visualized with AlexaFluor 488 conjugated secondary antibodies (Molecular Probes, Eugene, Oreg.) and mounted with DAPI (Invitrogen) to stain nuclear material. Images were acquired using: a Nikon TE2000S epifluorescence microscope, Nikon DsFi1 digital camera and Nikon NIS-Elements imaging software. Multiple images were taken at high resolution across the tumor and digitally merged to make a single margin-to-margin overview of the tumor. Images displayed in the manuscript are representative of the entire tumor and their respective experimental cohort.

### Western Blotting of Tumor Macrophages

Tumor cell suspensions were stained with antibodies specific for CD11b, IA (major histocompatibility complex (MHC) class II) and Gr1 as previously described (Gough et al., 2008, Cancer Res; 68:5206-15; Crittenden et al., 2012, PloS one; 7:e39295) and CD11b⁺Gr1^{lo}IA⁺ tumor macrophages were sorted using a BD Fluorescence Activated Cell Sorting (FACS) Aria Cell Sorter to greater than 98% purity. Cells were lysed in radioimmunoprecipitation assay (RIPA) buffer and denatured in sodium dodecyl sulfate (SDS) loading buffer containing β-mercaptoethanol, electrophoresed on 10% SDS-PAGE gels and transferred to nitrocellulose. Blocked blots were probed overnight at 4° C. with primary antibodies followed by horseradish peroxidase (HRP)-conjugated secondary antibodies. Binding was detected using a Pierce SuperSignal Pico Chemiluminescent Substrate (Thermo Fisher Scientific, Rockford, Ill.) and exposure to film.

### Flow Cytometry of Tumor, Blood and Lymph Nodes

For analysis of tumor-infiltrating cells, the tumor was dissected into approximately 2 mm fragments followed by agitation in lmg/mL collagenase (Invitrogen), 100μg/mL hyaluronidase (Sigma, St Louis, Mo.), and 20 mg/mL DNase (Sigma) in PBS for 1 hour at room temperature. The digest was filtered through 100 μm nylon mesh to remove macroscopic debris. For flow cytometry analysis of infiltrating cells, cell suspensions were washed and stained with directly conjugated fluorescent antibodies. For analysis of lymph nodes, lymph nodes were crushed, washed and surface stained, then cells were washed and fixed using a T regulatory cell staining kit (EBioscience) and stained for transcription factors. To measure cytokine responses, lymph node cells were plated to wells pre-coated with 1 μg/ml anti-CD3 for 4 hours in the presence of Golgiplug (BD biosciences). Cells were then surface stained, washed and fixed using a T regulatory cell staining kit (EBioscience) before intracellular cytokine staining. For analysis of cell numbers in blood, whole blood was harvested into ethylenediaminetetraacetic acid (EDTA) tubes from live mice via the saphenous vein, and 5-25 μl of fresh blood was stained directly with fluorescent antibody cocktails (see, Crittenden et al., PLoS One, 2013. 8(7): e69527). A known number of AccuCheck fluorescent beads (Invitrogen) were added to each sample, then red blood cells were lysed with Cal-Lyse whole blood lysing solution (Invitrogen), and samples analyzed on a BD LSRII flow cytometer. The absolute number of cells in the sample was determined based on comparing cellular events to bead events (cells/μl).

### Statistics

Data were analyzed and graphed using Prism (GraphPad Software, La Jolla, Calif.). Individual data sets were compared using Student's T-test. Analysis across multiple groups was performed using ANOVA with individual groups assessed using Tukey's comparison. Kaplan Meier survival curves were compared using a log-rank test.

| SEQ | | |
|---|---|---|
| ID NO | Description | Sequence |
| 1 | 9B12 VL | DIQMTQTTSSLSASLGDRVTISCRASQDISNYLNWYQQKPDGTVKLLIYYTSKLHSGVPSRFSG |
| | | SGSRTDYSLTITDLDQEDIATYFCQQGSALPWTFGQGTKVEIK |
| 2 | LCDR1 | RASQDISNYLN |
| 3 | LCDR2 | YTSKLHS |
| 4 | LCDR3 | QQGSALPWT |
| 5 | 9B12 VH | EVQLQESGPSLVKPSQTLSLTCSVTGDSFTSGYWNWIRKFPGNRLEYMGYISYNGITYHNPSL |
| | | KSRISITRDTSKNHYYLQLNSVTTEDTATYFCARYRYDYDGGHAMDYWGQGTLVTVSS |
| 6 | HFW1 | QVQLQESGPGLVKPSQTLSLTCAVYGGSFS |
| 7 | HFW1-variant | QVQLQESGPGLVKPSQTLSLTCAVYGDSFS |
| 8 | HCDR1 | SGYWN |
| 9 | HFW2-XXX | WIRX₃₉HPGKGLEX₄₇X₄₈G; where X₃₉ is Q or K, X₄₇ is W or Y, |
| | | and X₄₈ is I or M |
| 10 | HFW2-variant | WIRQHPGKGLEWIG |
| 11 | HFW2-variant | WIRKHPGKGLEYMG |
| 12 | HFW2-variant | WIRKHPGKGLEWIG |
| 13 | HFW2-variant | WIRKHPGKGLEYIG |
| 14 | HCDR2 | YISYNGITYHNPSLKS |
| 15 | HCDR2-variant | YISYNAITYHNPSLKS |
| 16 | HCDR2-variant | YISYSGITYHNPSLKS |
| 17 | HFW3-XXX | RITINX₇₁DTSKNQX₇₈SLQLNSVTPEDTAVYX₉₁CAR;, where X₇₁ is P or R, |
| | | X₇₈ is F or Y, and X₉₁ is Y or F |
| 18 | HFW3-variant | RITINPDTSKNQFSLQLNSVTPEDTAVYYCAR |
| 19 | HFW3-variant | RITINRDTSKNQYSLQLNSVTPEDTAVYFCAR |
| 20 | HFW3-variant | RITINRDTSKNQFSLQLNSVTPEDTAVYYCAR |
| 21 | HFW3-variant | RITINRDTSKNQFSLQLNSVTPEDTAVYFCAR |
| 22 | HFW3-variant | RITINRDTSKNQYSLQLNSVTPEDTAVYYCAR |
| 23 | HFW3-variant | RITINPDTSKNQYSLQLNSVTPEDTAVYFCAR |
| 24 | HFW3-variant | RITINPDTSKNQYSLQLNSVTPEDTAVYYCAR |
| 25 | HCDR3 | YRYDYDGGHAMDY |
| 26 | HCDR3-variant | YKYDYDAGHAMDY |
| 27 | HCDR3-variant | YKYDYDGGHAMDY |
| 28 | HFW4 | WGQGTLVTVSS |
| 29 | OX40mAb VL | DIQMTQSPSSLSASVGDRVTITCRASQDISNYLNWYQQKPGKAPKLLIYYTSKLHSGVPSRFSG |
| | | SGSGTDYTLTISSLQPEDFATYYCQQGSALPWTFGQGTKVEIK |
| 30 | OX40mAb light | DIQMTQSPSSLSASVGDRVTITCRASQDISNYLNWYQQKPGKAPKLLIYYTSKLHSGVPSRFSG |
| | chain | SGSGTDYTLTISSLQPEDFATYYCQQGSALPWTFGQGTKVEIKRTVAAPSVFIFPPSDEQLKSG |
| | | TASVVCLLNNFYPREAKVQWKVDNALQSGNSQESVTEQDSKDSTYSLSSTLTLSKADYEKHK |
| | | VYACEVTHQGLSSPVTKSFNRGEC |
| 31 | OX40Mab light | GACATCCAGATGACCCAGAGCCCCAGCAGCCTGAGCGCCAGCGTGGGCGACAGAGTGA |
| | chain DNA | CCATCACCTGTCGGGCCAGCCAGGACATCAGCAACTACCTGAACTGGTATCAGCAGAAG |
| | | CCCGGCAAGGCCCCCAAGCTGCTGATCTACTACACCAGCAAGCTGCACAGCGGCGTGCC |
| | | CAGCAGATTCAGCGGCAGCGGCTCCGGCACCGACTACACCCTGACCATCAGCAGCCTGC |
| | | AGCCCGAGGACTTCGCCACCTACTACTGCCAGCAGGGCTCCGCCCTGCCCTGGACCTTTG |
| | | GCCAGGGCACCAAGGTGGAAATCAAGCGTACGGTGGCTGCACCATCTGTCTTCATCTTCC |
| | | CGCCATCTGATGAGCAGTTGAAATCTGGAACTGCCTCTGTTGTGTGCCTGCTGAATAACT |
| | | TCTATCCCAGAGAGGCCAAAGTACAGTGGAAGGTGGATAACGCCCTCCAATCGGGTAAC |
| | | TCCCAGGAGAGTGTCACAGAGCAGGACAGCAAGGACAGCACCTACAGCCTCAGCAGCAC |
| | | CCTGACGCTGAGCAAAGCAGACTACGAGAAACACAAAGTCTACGCCTGCGAAGTCACCC |
| | | ATCAGGGCCTGAGCTCGCCCGTCACAAAGAGCTTCAACAGGGGAGAGTGT |
| 32 | OX40mAb VL-hu2 | DIQMTQSPSSLSASVGDRVTITCRASQDISNYLNWYQQKPGKAVKLLIYYTSKLHSGVPSRFS |
| | | GSGSRTDYTLTISSLQPEDFATYYCQQGSALPWTFGQGTKVEIK |
| 33 | OX40mAb5 VH | QVQLQESGPGLVKPSQTLSLTCAVYGGSFSSGYWNWIRQHPGKGLEWIGYISYNGITYHNPS |
| | | LKSRITINPDTSKNQFSLQLNSVTPEDTAVYYCARYRYDYDGGHAMDYWGQGTLVTVSS |
| 34 | OX40mAb5 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGCAGC |
| | | ACCCCGGCAAGGGCCTGGAATGGATCGGCTACATCAGCTACAACGGCATCACCTACCAC |
| | | AACCCCAGCCTGAAGTCCCGGATCACCATCAACCCCGACACCAGCAAGAACCAGTTCTCC |
| | | CTGCAGCTGAACAGCGTGACCCCCGAGGACACCGCCGTGTACTACTGCGCCCGGTACAG |
| | | ATACGACTACGACGGCGGCCACGCCATGGACTACTGGGGCCAGGGCACCCTGGTCACCG |
| | | TGTCCTCT |
| 35 | OX40mAb8 VH | QVQLQESGPGLVKPSQTLSLTCAVYGGSFSSGYWNWIRKHPGKGLEWIGYISYNGITYHNPS |
| | | LKSRITINRDTSKNQFSLQLNSVTPEDTAVYYCARYRYDYDGGHAMDYWGQGTLVTVSS |
| 36 | OX40mAb8VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATGGATCGGCTACATCAGCTACAACGGCATCACCTACCAC |
| | | AACCCCAGCCTGAAGTCCCGGATCACCATCAACCGGGACACCAGCAAGAACCAGTTCTCC |
| | | CTGCAGCTGAACAGCGTGACCCCCGAGGACACCGCCGTGTACTACTGCGCCCGGTACAG |
| | | ATACGACTACGACGGCGGCCACGCCATGGACTACTGGGGCCAGGGCACCCTGGTCACCG |
| | | TGTCCTCT |
| 37 | OX40mAb13 VH | QVQLQESGPGLVKPSQTLSLTCAVYGDSFSSGYWNWIRKHPGKGLEYMGYISYNGITYHNPS |
| | | LKSRITINRDTSKNQYSLQLNSVTPEDTAVYFCARYRYDYDGGHAMDYWGQGTLVTVSS |
| 38 | OX40mAb13 VH | CAGGTGCAGCTGCAGGAAAGCGGCCCTGGCCTGGTCAAGCCCAGCCAGACCCTGAGCCT |
| | DNA | GACCTGTGCCGTGTACGGCGACAGCTTCAGCAGCGGCTACTGGAACTGGATCCGGAAGC |
| | | ACCCCGGCAAGGGCCTGGAATACATGGGCTACATCAGCTACAACGGCATCACCTACCAC |
| | | AACCCCAGCCTGAAGTCCCGGATCACCATCAACCGGGACACCAGCAAGAACCAGTACTCC |
| | | CTGCAGCTGAACAGCGTGACCCCCGAGGACACCGCCGTGTACTTCTGCGCCCGGTACAG |
| | | ATACGACTACGACGGCGGCCACGCCATGGACTACTGGGGCCAGGGCACCCTGGTCACCG |
| | | TGTCCTCT |
| 39 | OX40mAb14 VH | QVQLQESGPGLVKPSQTLSLTCAVYGDSFSSGYWNWIRKHPGKGLEYIGYISYNGITYHNPS |
| | | LKSRITINRDTSKNQYSLQLNSVTPEDTAVYFCARYRYDYDGGHAMDYWGQGTLVTVSS |
| 40 | OX40mAb14 VH | CAGGTGCAGCTGCAGGAAAGCGGCCCTGGCCTGGTCAAGCCCAGCCAGACCCTGAGCCT |
| | DNA | GACCTGTGCCGTGTACGGCGACAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTTCTGCGCCCGGTACAGA |
| | | TACGACTACGACGGCGGCCACGCCATGGACTACTGGGGCCAGGGCACCCTGGTCACCGT |
| | | GTCCTCT |
| 41 | OX40mAb15 VH | QVQLQESGPGLVKPSQTLSLTCAVYGDSFSSGYWNWIRKHPGKGLEYIGYISYNGITYHNPS |
| | | LKSRITINRDTSKNQFSLQLNSVTPEDTAVYFCARYRYDYDGGHAMDYWGQGTLVTVSS |
| 42 | OX40mAb15 VH | CAGGTGCAGCTGCAGGAAAGCGGCCCTGGCCTGGTCAAGCCCAGCCAGACCCTGAGCCT |
| | DNA | GACCTGTGCCGTGTACGGCGACAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTTCTCCC |
| | | TGCAGCTGAACAGCGTGACCCCCGAGGACACCGCCGTGTACTTCTGCGCCCGGTACAGA |
| | | TACGACTACGACGGCGGCCACGCCATGGACTACTGGGGCCAGGGCACCCTGGTCACCGT |
| | | GTCCTCT |
| 43 | OX40mAb16 VH | QVQLQESGPGLVKPSQTLSLTCAVYGDSFSSGYWNWIRKHPGKGLEYIGYISYNGITYHNPS |
| | | LKSRITINRDTSKNQYSLQLNSVTPEDTAVYYCARYRYDYDGGHAMDYWGQGTLVTVSS |
| 44 | OX40mAb16 VH | CAGGTGCAGCTGCAGGAAAGCGGCCCTGGCCTGGTCAAGCCCAGCCAGACCCTGAGCCT |
| | DNA | GACCTGTGCCGTGTACGGCGACAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAGA |
| | | TACGACTACGACGGCGGCCACGCCATGGACTACTGGGGCCAGGGCACCCTGGTCACCGT |
| | | GTCCTCT |
| 45 | OX40mAb17 VH | QVQLQESGPGLVKPSQTLSLTCAVYGDSFSSGYWNWIRKHPGKGLEYIGYISYNGITYHNPS |
| | | LKSRITINRDTSKNQFSLQLNSVTPEDTAVYYCARYRYDYDGGHAMDYWGQGTLVTVSS |
| 46 | OX40mAb VH17 | CAGGTGCAGCTGCAGGAAAGCGGCCCTGGCCTGGTCAAGCCCAGCCAGACCCTGAGCCT |
| | DNA | GACCTGTGCCGTGTACGGCGACAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTTCTCCC |
| | | TGCAGCTGAACAGCGTGACCCCCGAGGACACCGCCGTGTACTACTGCGCCCGGTACAGA |
| | | TACGACTACGACGGCGGCCACGCCATGGACTACTGGGGCCAGGGCACCCTGGTCACCGT |
| | | GTCCTCT |
| 47 | OX40mAb18 VH | QVQLQESGPGLVKPSQTLSLTCAVYGGSFSSGYWNWIRKHPGKGLEYIGYISYNGITYHNPS |
| | | LKSRITINPDTSKNQYSLQLNSVTPEDTAVYFCARYRYDYDGGHAMDYWGQGTLVTVSS |
| 48 | OX40mAb18 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCCCGACACCAGCAAGAACCAGTACTCCC |
| | | TGCAGCTGAACAGCGTGACCCCCGAGGACACCGCCGTGTACTTCTGCGCCCGGTACAGA |
| | | TACGACTACGACGGCGGCCACGCCATGGACTACTGGGGCCAGGGCACCCTGGTCACCGT |
| | | GTCCTCT |
| 49 | OX40mAb19 VH | QVQLQESGPGLVKPSQTLSLTCAVYGGSFSSGYWNWIRKHPGKGLEYIGYISYNGITYHNPS |
| | | LKSRITINRDTSKNQYSLQLNSVTPEDTAVYFCARYRYDYDGGHAMDYWGQGTLVTVSS |
| 50 | OX40mAb19 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTTCTGCGCCCGGTACAGA |
| | | TACGACTACGACGGCGGCCACGCCATGGACTACTGGGGCCAGGGCACCCTGGTCACCGT |
| | | GTCCTCT |
| 51 | OX40mAb20 VH | QVQLQESGPGLVKPSQTLSLTCAVYGGSFSSGYWNWIRKHPGKGLEYIGYISYNGITYHNPS |
| | | LKSRITINRDTSKNQYSLQLNSVTPEDTAVYYCARYRYDYDGGHAMDYWGQGTLVTVSS |
| 52 | OX40mAb20 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAGA |
| | | TACGACTACGACGGCGGCCACGCCATGGACTACTGGGGCCAGGGCACCCTGGTCACCGT |
| | | GTCCTCT |
| 53 | OX40mAb21 VH | QVQLQESGPGLVKPSQTLSLTCAVYGGSFSSGYWNWIRKHPGKGLEYIGYISYNGITYHNPS |
| | | LKSRITINPDTSKNQYSLQLNSVTPEDTAVYYCARYRYDYDGGHAMDYWGQGTLVTVSS |
| 54 | OX40mAb21 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCCCGACACCAGCAAGAACCAGTACTCCC |
| | | TGCAGCTGAACAGCGTGACCCCCGAGGACACCGCCGTGTACTACTGCGCCCGGTACAGA |
| | | TACGACTACGACGGCGGCCACGCCATGGACTACTGGGGCCAGGGCACCCTGGTCACCGT |
| | | GTCCTCT |
| 55 | OX40mAb22 VH | QVQLQESGPGLVKPSQTLSLTCAVYGGSFSSGYWNWIRKHPGKGLEYIGYISYNAITYHNPS |
| | | LKSRITINRDTSKNQYSLQLNSVTPEDTAVYYCARYKYDYDAGHAMDYWGQGTLVTVSS |
| 56 | OX40mAb22 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGCCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGCCGGCCACGCCATGGACTACTGGGGCCAGGGCACCCTGGTCACCGT |
| | | GTCCTCT |
| 57 | OX40mAb23 VH | QVQLQESGPGLVKPSQTLSLTCAVYGGSFSSGYWNWIRKHPGKGLEYIGYISYNAITYHNPS |
| | | LKSRITINRDTSKNQYSLQLNSVTPEDTAVYYCARYRYDYDGGHAMDYWGQGTLVTVSS |
| 58 | OX40mAb23 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGCCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAGA |
| | | TACGACTACGACGGCGGCCACGCCATGGACTACTGGGGCCAGGGCACCCTGGTCACCGT |
| | | GTCCTCT |
| 59 | OX40mAb24 VH | QVQLQESGPGLVKPSQTLSLTCAVYGGSFSSGYWNWIRKHPGKGLEYIGYISYNGITYHNPS |
| | | LKSRITINRDTSKNQYSLQLNSVTPEDTAVYYCARYKYDYDGGHAMDYWGQGTLVTVSS |
| 60 | OX40mAb24 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCT |
| 61 | OX40mAb25 VH | QVQLQESGPGLVKPSQTLSLTCAVYGGSFSSGYWNWIRKHPGKGLEYIGYISYSGITYHNPS |
| | | LKSRITINRDTSKNQYSLQLNSVTPEDTAVYYCARYRYDYDGGHAMDYWGQGTLVTVSS |
| 62 | OX40mAb25 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAGCGGCATCACCTACCAC |
| | | AACCCCAGCCTGAAGTCCCGGATCACCATCAACCGGGACACCAGCAAGAACCAGTACTCC |
| | | CTGCAGCTGAACAGCGTGACCCCCGAGGACACCGCCGTGTACTACTGCGCCCGGTACAG |
| | | ATACGACTACGACGGCGGCCACGCCATGGACTACTGGGGCCAGGGCACCCTGGTCACCG |
| | | TGTCCTCT |
| 63 | OX40mAb25a VH | QVQLQESGPGLVKPSQTLSLTCAVYGGSFSSGYWNWIRKHPGKGLEYIGYISYSGITYHNPS |
| | | LKSRITINRDTSKNQYSLQLNSVTPEDTAVYYCARYKYDYDGGHAMDYWGQGTLVTVSS |
| 64 | OX40mAb25a VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAGCGGCATCACCTACCAC |
| | | AACCCCAGCCTGAAGTCCCGGATCACCATCAACCGGGACACCAGCAAGAACCAGTACTCC |
| | | CTGCAGCTGAACAGCGTGACCCCCGAGGACACCGCCGTGTACTACTGCGCCCGGTACAA |
| | | ATACGACTACGACGGCGGCCACGCCATGGACTACTGGGGCCAGGGCACCCTGGTCACCG |
| | | TGTCCTCT |
| 65 | OX40mAb26 VH | EVQLQESGPSLVKPSQTLSLTCSVTGDSFTSGYWNWIRKFPGNRLEYMGYISYNAITYHNPS |
| | | LKSRISITRDTSKNHYYLQLNSVTTEDTATYFCARYRYDYDGGHAMDYWGQGTLVTVSS |
| 66 | OX40mAb26 VH | GAGGTGCAGCTGCAGGAAAGCGGCCCCAGCCTGGTCAAGCCCAGCCAGACCCTGAGCCT |
| | DNA | GACCTGCAGCGTGACCGGCGACAGCTTCACCAGCGGCTACTGGAACTGGATCCGGAAGT |
| | | TCCCCGGCAACCGGCTCGAGTACATGGGCTACATCAGCTACAACGCCATCACCTACCACA |
| | | ACCCCAGCCTGAAGTCCCGGATCAGCATCACCCGGGACACCAGCAAGAACCACTACTACC |
| | | TGCAGCTGAACAGCGTGACCACCGAGGACACCGCCACCTACTTTTGCGCCCGGTACAGAT |
| | | ACGACTACGACGGCGGCCACGCCATGGACTACTGGGGCCAGGGCACCCTGGTCACCGTG |
| | | TCCTCT |
| 67 | OX40mAb27 VH | QVQLQESGPGLVKPSQTLSLTCAVYGGSFSSGYWNWIRKHPGKGLEYIGYISYNAITYHNPS |
| | | LKSRITINRDTSKNQYSLQLNSVTPEDTAVYYCARYKYDYDGGHAMDYWGQGTLVTVSS |
| 68 | OX40mA27 VH | CAGGTGCAGCTGCAGGAAAGCGGCCCTGGCCTGGTCAAGCCCAGCCAGACCCTGAGCCT |
| | DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGCCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCT |
| 69 | Human IgG1 CH | ASTKGPSVFPLAPSSKSTSGGTAALGCLVKDYFPEPVTVSWNSGALTSGVHTFPAVLQSSGLY |
| | chain | SLSSVVTVPSSSLGTQTYICNVNHKPSNTKVDKRVEPKSCDKTHTCPPCPAPELLGGPSVFLFP |
| | | PKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHE |
| | | ALHNHYTQKSLSLSPGK |
| 70 | Human IgG1 CH | GCgTCgACCAAGGGCCCATCcGTCTTCCCCCTGGCACCCTCCTCCAAGAGCACCTCTGGGG |
| | chain DNA | GCACAGCGGCCCTGGGCTGCCTGGTCAAGGACTACTTCCCCGAACCGGTGACGGTGTCcT |
| | | GGAACTCAGGCGCtCTGACCAGCGGCGTGCACACCTTCCCGGCTGTCCTACAGTCCTCAG |
| | | GACTCTACTCCCTCAGCAGCGTGGTGACCGTGCCCTCCAGCAGCTTGGGCACCCAGACCT |
| | | ACATCTGCAACGTGAATCACAAGCCCAGCAACACCAAGGTGGACAAGAGAGTTGAGCCC |
| | | AAATCTTGTGACAAAACTCACACATGCCCACCGTGCCCAGCACCTGAACTCCTGGGGGGA |
| | | CCGTCAGTCTTCCTCTTCCCCCCAAAACCCAAGGACACCCTCATGATCTCCCGGACCCCTG |
| | | AGGTCACATGCGTGGTGGTGGACGTGAGCCACGAAGACCCTGAGGTCAAGTTCAACTGG |
| | | TACGTGGACGGCGTGGAGGTGCATAATGCCAAGACAAAGCCGCGGGAGGAGCAGTACA |
| | | ACAGCACGTACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGAGAAAACCAT |
| | | CTCCAAAGCCAAAGGGCAGCCCCGAGAACCACAGGTcTACACCCTGCCCCCATCCCGGGA |
| | | GGAGATGACCAAGAACCAGGTCAGCCTGACCTGCCTGGTCAAAGGCTTCTATCCCAGCG |
| | | ACATCGCCGTGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACCGTGGACAAGAG |
| | | CAGGTGGCAGCAGGGGAACGTCTTCTCATGCTCCGTGATGCATGAGGCTCTGCACAACC |
| | | ACTACACGCAGAAGAGCttaagCCTGTCTCCGGGTAAA |
| 71 | OX40mAb24 heavy | QVQLQESGPGLVKPSQTLSLTCAVYGGSFSSGYWNWIRKHPGKGLEYIGYISYNGITYHNPSL |
| | chain | KSRITINRDTSKNQYSLQLNSVTPEDTAVYYCARYKYDYDGGHAMDYWGQGTLVTVSSASTK |
| | | GPSVFPLAPSSKSTSGGTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPELLGGPSVFLFPPKPKD |
| | | TLMISRTPEVTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQGNVFSCSVMHEALHN |
| | | HYTQKSLSLSPGK |
| 72 | OX40mAb24 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCgTCgACCAAGGGCCCATCcGTCTTCCCCCTGGCACCCTCCTCCAAGAGCACCT |
| | | CTGGGGGCACAGCGGCCCTGGGCTGCCTGGTCAAGGACTACTTCCCCGAACCGGTGACG |
| | | GTGTCcTGGAACTCAGGCGCtCTGACCAGCGGCGTGCACACCTTCCCGGCTGTCCTACAG |
| | | TCCTCAGGACTCTACTCCCTCAGCAGCGTGGTGACCGTGCCCTCCAGCAGCTTGGGCACC |
| | | CAGACCTACATCTGCAACGTGAATCACAAGCCCAGCAACACCAAGGTGGACAAGAGAGT |
| | | TGAGCCCAAATCTTGTGACAAAACTCACACATGCCCACCGTGCCCAGCACCTGAACTCCT |
| | | GGGGGGACCGTCAGTCTTCCTCTTCCCCCCAAAACCCAAGGACACCCTCATGATCTCCCG |
| | | GACCCCTGAGGTCACATGCGTGGTGGTGGACGTGAGCCACGAAGACCCTGAGGTCAAGT |
| | | TCAACTGGTACGTGGACGGCGTGGAGGTGCATAATGCCAAGACAAAGCCGCGGGAGGA |
| | | GCAGTACAACAGCACGTACCGTGTGGTCAGCGTCCTCACCGTCCTGCACCAGGACTGGCT |
| | | GAATGGCAAGGAGTACAAGTGCAAGGTCTCCAACAAAGCCCTCCCAGCCCCCATCGAGA |
| | | AAACCATCTCCAAAGCCAAAGGGCAGCCCCGAGAACCACAGGTcTACACCCTGCCCCCAT |
| | | CCCGGGAGGAGATGACCAAGAACCAGGTCAGCCTGACCTGCCTGGTCAAAGGCTTCTAT |
| | | CCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTATAGCAAGCTCACCGTGGA |
| | | CAAGAGCAGGTGGCAGCAGGGGAACGTCTTCTCATGCTCCGTGATGCATGAGGCTCTGC |
| | | ACAACCACTACACGCAGAAGAGCttaagCCTGTCTCCGGGTAAA |
| 73 | OX40mAb28 heavy | QVQLQESGPGLVKPSQTLSLTCAVYGGSFSSGYWNWIRKHPGKGLEYIGYISYNGITYHNPSL |
| | chain | KSRITINRDTSKNQYSLQLNSVTPEDTAVYYCARYKYDYDGGHAMDYWGQGTLVTVSSASTK |
| | | GPSVFPLAPCSRSTSESTAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEGNVFSCSVMHEALHNHY |
| | | TQKSLSLSLGK |
| 74 | OX40mAb28 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCGTCGACCAAGGGCCCCAGCGTGTTCCCCCTGGCCCCTTGCAGCAGAAGCAC |
| | | CAGCGAGAGCACAGCCGCCCTGGGCTGCCTGGTGAAGGACTACTTCCCCGAGCCCGTGA |
| | | CCGTGTCCTGGAACAGCGGCGCTCTGACCAGCGGCGTGCATACCTTCCCCGCCGTGCTCC |
| | | AGAGCAGCGGACTGTACTCCCTGAGCAGCGTGGTGACCGTGCCTTCCAGCAGCCTGGGC |
| | | ACCAAGACCTACACCTGCAACGTGGACCACAAGCCCAGCAACACCAAGGTGGACAAGAG |
| | | AGTGGAGAGCAAGTACGGCCCTCCCTGCCCCCCTTGCCCTGCCCCCGAGTTCCTGGGCGG |
| | | ACCTAGCGTGTTCCTGTTCCCCCCCAAGCCCAAGGACACCCTGATGATCAGCAGAACCCC |
| | | CGAGGTGACCTGCGTGGTGGTGGACGTGTCCCAGGAGGACCCCGAGGTCCAGTTTAATT |
| | | GGTACGTGGACGGCGTGGAAGTGCATAACGCCAAGACCAAGCCCAGAGAGGAGCAGTT |
| | | CAACAGCACCTACAGAGTGGTGTCCGTGCTGACCGTGCTGCACCAGGACTGGCTGAACG |
| | | GCAAGGAATACAAGTGCAAGGTCTCCAACAAGGGCCTGCCTAGCAGCATCGAGAAGACC |
| | | ATCAGCAAGGCCAAGGGCCAGCCACGGGAGCCCCAGGTCTACACCCTGCCACCTAGCCA |
| | | AGAGGAGATGACCAAGAACCAGGTGTCCCTGACCTGTCTGGTGAAAGGCTTCTATCCCA |
| | | GCGATATCGCCGTGGAGTGGGAGAGCAACGGCCAGCCCGAGAACAACTACAAGACCAC |
| | | CCCCCCTGTGCTGGACAGCGACGGCAGCTTCTTCCTGTACTCCAGACTGACCGTGGACAA |
| | | GTCCAGATGGCAGGAGGGCAACGTCTTCAGCTGCTCCGTGATGCACGAGGCCCTGCACA |
| | | ACCACTACACCCAGAAGTCCCTGAGCCTGAGCCTGGGCAAG |
| 75 | OX40mAb29 heavy | QVQLQESGPGLVKPSQTLSLTCAVYGGSFSSGYWNWIRKHPGKGLEYIGYISYNGITYHNPSL |
| | chain | KSRITINRDTSKNQYSLQLNSVTPEDTAVYYCARYKYDYDGGHAMDYWGQGTLVTVSSASTK |
| | | GPSVFPLAPSSKSTSGGTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPEFEGGPSVFLFPPKPK |
| | | DTLMISRTPEVTCVVVDVSHEDPEVKFNWYVDGVEVHNAKTKPREEQYNSTYRVVSVLTVL |
| | | HQDWLNGKEYKCKVSNKALPASIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSCSVMHEALH |
| | | NHYTQKSLSLSPGK |
| 76 | OX40mAb29 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCGTCGACCAAGGGCCCATCCGTCTTCCCCCTGGCACCCTCCTCCAAGAGCACC |
| | | TCTGGGGGCACAGCGGCCCTGGGCTGCCTGGTCAAGGACTACTTCCCCGAACCGGTGAC |
| | | GGTGTCcTGGAACTCAGGCGCtCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAAGAGAG |
| | | TTGAGCCCAAATCTTGTGACAAAACTCACACATgcCCacCGTGCCCAGCACCTGAATTCGA |
| | | GGGGGGAcCGTCAGTCTTCCTCTTCCCCCCAAAACCCaaGgACACCCTCATGATCTCCCGG |
| | | ACCCCTGAGGTCACATGCGTGGTGGTGGACGTGAGCCACGAAGACCCTGAGGTCAAGTT |
| | | CAACTGGTACGTGGACGGCGTGGAGGTGCATAATGCCAAGACAAAGCCGCGGGAGGAG |
| | | CAGTACAACAGCACGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGCCTCCATCGAGAA |
| | | AACCATCTCCAAAGCCAAAGGGCAGCCCCGAGAACCACAGGTcTACACCCTGCCCCCATC |
| | | CCGGGAGGAGATGACCAAGAACCAGGTCAGCCTGACCTGCCTGGTCAAAGGCTTCTATC |
| | | CCAGCGACATCGCCGTGGAGTGGGAGAGCAATGGGCAGCCGGAGAACAACTACAAGAC |
| | | CACGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCTCCGGGTAAA |
| 77 | OX40mAb31 heavy | QVQLQESGPGLVKPSQTLSLTCAVYGGSFSSGYWNWIRKHPGKGLEYIGYISYNAITYHNPSL |
| | chain | KSRITINRDTSKNQYSLQLNSVTPEDTAVYYCARYKYDYDGGHAMDYWGQGTLVTVSSASTK |
| | | GPSVFPLAPCSRSTSESTAALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEGNVFSCSVMHEALHNHY |
| | | TQKSLSLSLGK |
| 78 | OX40mAb31 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGCCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCGTCGACCAAGGGCCCCAGCGTGTTCCCCCTGGCCCCTTGCAGCAGAAGCAC |
| | | CAGCGAGAGCACAGCCGCCCTGGGCTGCCTGGTGAAGGACTACTTCCCCGAGCCCGTGA |
| | | CCGTGTCCTGGAACAGCGGCGCTCTGACCAGCGGCGTGCATACCTTCCCCGCCGTGCTCC |
| | | AGAGCAGCGGACTGTACTCCCTGAGCAGCGTGGTGACCGTGCCTTCCAGCAGCCTGGGC |
| | | ACCAAGACCTACACCTGCAACGTGGACCACAAGCCCAGCAACACCAAGGTGGACAAGAG |
| | | AGTGGAGAGCAAGTACGGCCCTCCCTGCCCCCCTTGCCCTGCCCCCGAGTTCCTGGGCGG |
| | | ACCTAGCGTGTTCCTGTTCCCCCCCAAGCCCAAGGACACCCTGATGATCAGCAGAACCCC |
| | | CGAGGTGACCTGCGTGGTGGTGGACGTGTCCCAGGAGGACCCCGAGGTCCAGTTTAATT |
| | | GGTACGTGGACGGCGTGGAAGTGCATAACGCCAAGACCAAGCCCAGAGAGGAGCAGTT |
| | | CAACAGCACCTACAGAGTGGTGTCCGTGCTGACCGTGCTGCACCAGGACTGGCTGAACG |
| | | GCAAGGAATACAAGTGCAAGGTCTCCAACAAGGGCCTGCCTAGCAGCATCGAGAAGACC |
| | | ATCAGCAAGGCCAAGGGCCAGCCACGGGAGCCCCAGGTCTACACCCTGCCACCTAGCCA |
| | | AGAGGAGATGACCAAGAACCAGGTGTCCCTGACCTGTCTGGTGAAAGGCTTCTATCCCA |
| | | GCGATATCGCCGTGGAGTGGGAGAGCAACGGCCAGCCCGAGAACAACTACAAGACCAC |
| | | CCCCCCTGTGCTGGACAGCGACGGCAGCTTCTTCCTGTACTCCAGACTGACCGTGGACAA |
| | | GTCCAGATGGCAGGAGGGCAACGTCTTCAGCTGCTCCGTGATGCACGAGGCCCTGCACA |
| | | ACCACTACACCCAGAAGTCCCTGAGCCTGAGCCTGGGCAAG |
| 79 | OX40mAb32 heavy | QVQLQESGPGLVKPSQTLSLTCAVYGGSFSSGYWNWIRKHPGKGLEYIGYISYNAITYHNPSL |
| | chain | KSRITINRDTSKNQYSLQLNSVTPEDTAVYYCARYKYDYDGGHAMDYWGQGTLVTVSSASTK |
| | | GPSVFPLAPSSKSTSGGTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPEFEGGPSVFLFPPKPK |
| | | DTLMISRTPEVTCVVVDVSHEDPEVKFNWYVDGVEVHNAKTKPREEQYNSTYRVVSVLTVL |
| | | HQDWLNGKEYKCKVSNKALPASIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSCSVMHEALH |
| | | NHYTQKSLSLSPGK |
| 80 | OX40mAb32 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGCCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCGTCGACCAAGGGCCCATCCGTCTTCCCCCTGGCACCCTCCTCCAAGAGCACC |
| | | TCTGGGGGCACAGCGGCCCTGGGCTGCCTGGTCAAGGACTACTTCCCCGAACCGGTGAC |
| | | GGTGTCcTGGAACTCAGGCGCtCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTGGGCAC |
| | | CCAGACCTACATCTGCAACGTGAATCACAAGCCCAGCAACACCAAGGTGGACAAGAGAG |
| | | TTGAGCCCAAATCTTGTGACAAAACTCACACATgcCCacCGTGCCCAGCACCTGAATTCGA |
| | | GGGGGGAcCGTCAGTCTTCCTCTTCCCCCCAAAACCCaaGgACACCCTCATGATCTCCCGG |
| | | ACCCCTGAGGTCACATGCGTGGTGGTGGACGTGAGCCACGAAGACCCTGAGGTCAAGTT |
| | | CAACTGGTACGTGGACGGCGTGGAGGTGCATAATGCCAAGACAAAGCCGCGGGAGGAG |
| | | CAGTACAACAGCACGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGCCTCCATCGAGAA |
| | | AACCATCTCCAAAGCCAAAGGGCAGCCCCGAGAACCACAGGTcTACACCCTGCCCCCATC |
| | | CCGGGAGGAGATGACCAAGAACCAGGTCAGCCTGACCTGCCTGGTCAAAGGCTTCTATC |
| | | CCAGCGACATCGCCGTGGAGTGGGAGAGCAATGGGCAGCCGGAGAACAACTACAAGAC |
| | | CACGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCTCCGGGTAAA |
| 81 | OX40mAb37 heavy | QVQLQESGPGLVKPSQTLSLTCAVYGGSFSSGYWNWIRKHPGKGLEYIGYISYNGITYHNPSL |
| | chain | KSRITINRDTSKNQYSLQLNSVTPEDTAVYYCARYKYDYDGGHAMDYWGQGTLVTVSSAKTT |
| | | PPSVYPLAPGSAAQTNSMVTLGCLVKGYFPEPVTVTWNSGSLSSGVHTFPAVLQSDLYTLSSS |
| | | VTVPSSTWPSETVTCNVAHPASSTKVDKKIVPRDCGCKPCICTVPEVSSVFIFPPKPKDVLTI |
| | | TLTPKVTCVVVDISKDDPEVQFSWFVDDVEVHTAQTQPREEQFNSTFRSVSELPIMHQDWLNG |
| | | KEFKCRVNSAAFPAPIEKTISKTKGRPKAPQVYTIPPPKEQMAKDKVSLTCMITDFFPEDITV |
| | | EWQWNGQPAENYKNTQPIMDTDGSYFVYSKLNVQKSNWEAGNTFTCSVLHEGLHNHHTEK |
| | | SLSHSPGK |
| 82 | OX40mAb37 heavy | CAGGTGCAGCTGCAGGAAAGCGGCCCTGGCCTGGTCAAGCCCAGCCAGACCCTGAGCCT |
| | chain DNA | GACCTGTGCCGTGTACGGCGGCAGCTTCAGCAGCGGCTACTGGAACTGGATCCGGAAGC |
| | | ACCCCGGCAAGGGCCTGGAATACATCGGCTACATCAGCTACAACGGCATCACCTACCACA |
| | | ACCCCAGCCTGAAGTCCCGGATCACCATCAACCGGGACACCAGCAAGAACCAGTACTCCC |
| | | TGCAGCTGAACAGCGTGACCCCCGAGGACACCGCCGTGTACTACTGCGCCCGGTACAAA |
| | | TACGACTACGACGGCGGCCACGCCATGGACTACTGGGGCCAGGGCACCCTGGTCACCGT |
| | | GTCCTCTGCGaaGACGACACCCCCATCTGTCTATCCACTGGCCCCTGGATCTGCTGCCCAA |
| | | ACTAACTCCATGGTGACCCTGGGATGCCTGGTCAAGGGCTATTTCCCTGAGCCAGTGACA |
| | | GTGACCTGGAACTCTGGATCCCTGTCCAGCGGTGTGCACACCTTCCCAGCTGTCCTGCAG |
| | | TCTGACCTCTACACTCTGAGCAGCTCAGTGACTGTCCCCTCCAGCACCTGGCCCAGCGAG |
| | | ACCGTCACCTGCAACGTTGCCCACCCGGCCAGCAGCACCAAGGTGGACAAGAAAATTGT |
| | | GCCCAGGGATTGTGGTTGTAAGCCTTGCATATGTACcGTCCCAGAAGTATCATCTGTCTTC |
| | | ATCTTCCCCCCAAAGCCCAAGGATGTGCTCACCATTACTCTGACTCCTAAGGTCACGTGTG |
| | | TTGTGGTAGACATCAGCAAGGATGATCCCGAGGTCCAGTTCAGCTGGTTTGTAGATGAT |
| | | GTGGAGGTGCACACAGCTCAGACGCAACCCCGGGAGGAGCAGTTCAACAGCACTTTCCG |
| | | CTCAGTCAGTGAACTTCCCATCATGCACCAGGACTGGCTCAATGGCAAGGAGTTCAAATG |
| | | CAGGGTCAACAGTGCAGCTTTCCCTGCCCCCATCGAGAAAACCATCTCCAAAACCAAAGG |
| | | CAGACCGAAGGCTCCACAGGTGTAtACCATTCCACCTCCCAAGGAGCAGATGGCCAAGG |
| | | ATAAAGTCAGTCTGACCTGCATGATAACAGACTTCTTCCCTGAAGACATTACTGTGGAGT |
| | | GGCAGTGGAATGGGCAGCCAGCGGAGAACTACAAGAACACTCAGCCCATCATGGACAC |
| | | AGATGGCTCTTACTTCGTCTACAGCAAGCTCAATGTGCAGAAGAGCAACTGGGAGGCAG |
| | | GAAATACTTTCACCTGCTCTGTGTTACATGAGGGCCTGCACAACCACCATACTGAGAAGA |
| | | GCCTCTCCCACTCTCCTGGTAAA |
| 83 | OX40mAb37 light | DIQMTQSPSSLSASVGDRVTITCRASQDISNYLNWYQQKPGKAPKLLIYYTSKLHSGVPSRF |
| | chain | SGSGSGTDYTLTISSLQPEDFATYYCQQGSALPWTFGQGTKVEIKRADAAPTVSIFPPSSEQ |
| | | LTSGGASVVCFLNNFYPKDINVKWKIDGSERQNGVLNSWTDQDSKDSTYSMSSTLTLTKDEY |
| | | ERHNSYTCEATHKTSTSPIVKSFNRNEC |
| 84 | OX40mAb37 light | GACATCCAGATGACCCAGAGCCCCAGCAGCCTGAGCGCCAGCGTGGGCGACAGAGTGA |
| | chain DNA | CCATCACCTGTCGGGCCAGCCAGGACATCAGCAACTACCTGAACTGGTATCAGCAGAAG |
| | | CCCGGCAAGGCCCCCAAGCTGCTGATCTACTACACCAGCAAGCTGCACAGCGGCGTGCC |
| | | CAGCAGATTCAGCGGCAGCGGCTCCGGCACCGACTACACCCTGACCATCAGCAGCCTGC |
| | | AGCCCGAGGACTTCGCCACCTACTACTGCCAGCAGGGCTCCGCCCTGCCCTGGACCTTTG |
| | | GCCAGGGCACCAAGGTGGAAATCAAGCGGGCTGATGCGGCGCCAACTGTATCCATCTTC |
| | | CCACCATCCAGTGAGCAGTTAACATCTGGAGGTGCCTCAGTCGTGTGCTTCTTGAACAAC |
| | | TTCTACCCCAAAGACATCAATGTCAAGTGGAAGATTGATGGCAGTGAACGACAAAATGG |
| | | CGTCCTGAACAGTTGGACTGATCAGGACAGCAAAGACAGCACCTACAGCATGAGCAGCA |
| | | CCCTCACGTTGACCAAGGACGAGTATGAACGACATAACAGCTATACCTGTGAGGCCACTC |
| | | ACAAGACATCAACTTCACCCATTGTCAAGAGCTTCAACAGGAATGAGTGT |
| 85 | OX86 VH | QVQLKESGPGLVQPSQTLSLTCTVSGFSLTGYNLHWVRQPPGKGLEWMGRMRYDGDTYYN |
| | | SVLKSRLSISRDTSKNQVFLKMNSLQTDDTAIYYCTRDGRGDSFDYWGQGVMVTVSS |
| 86 | OX86 heavy chain | QVQLKESGPGLVQPSQTLSLTCTVSGFSLTGYNLHWVRQPPGKGLEWMGRMRYDGDTYYN |
| | | SVLKSRLSISRDTSKNQVFLKMNSLQTDDTAIYYCTRDGRGDSFDYWGQGVMVTVSSASTTP |
| | | PSVYPLAPGSAAQTNSMVTLGCLVKGYFPEPVTVTWNSGSLSSGVHTFPAVLQSDLYTLSSSV |
| | | TVPSSTWPSETVTCNVAHPASSTKVDKKIVPRDCGCKPCICTVPEVSSVFIFPPKPKDVLTIT |
| | | LTPKVTCVVVDISKDDPEVQFSWFVDDVEVHTAQTQPREEQFNSTFRSVSELPIMHQDWLNGK |
| | | EFKCRVNSAAFPAPIEKTISKTKGRPKAPQVYTIPPPKEQMAKDKVSLTCMITDFFPEDITVE |
| | | WQWNGQPAENYKNTQPIMDTDGSYFVYSKLNVQKSNWEAGNTFTCSVLHEGLHNHHTEKSL |
| | | SHSPGK |
| 87 | OX86 heavy chain | CAGGTGCAGCTGAAGGAGTCAGGACCTGGTCTGGTGCAGCCCTCACAGACCCTGTCCCT |
| | DNA | CACCTGCACTGTCTCTGGGTTCTCACTAACCGGTTACAATTTACACTGGGTTCGCCAGCCT |
| | | CCAGGAAAGGGTCTGGAGTGGATGGGAAGAATGAGGTATGATGGAGACACATATTATA |
| | | ATTCAGTTCTCAAATCCCGACTGAGCATCAGCAGGGACACCTCCAAGAACCAAGTTTTCTT |
| | | GAAAATGAACAGTCTGCAAACGGATGACACAGCCATTTACTATTGTACCAGAGACGGGC |
| | | GTGGTGACTCCTTTGATTACTGGGGCCAAGGAGTCATGGTCACAGTCTCCTCCGCGTCGA |
| | | CGACACCCCCATCTGTCTATCCACTGGCCCCTGGATCTGCTGCCCAAACTAACTCCATGGT |
| | | GACCCTGGGATGCCTGGTCAAGGGCTATTTCCCTGAGCCAGTGACAGTGACCTGGAACT |
| | | CTGGATCCCTGTCCAGCGGTGTGCACACCTTCCCAGCTGTCCTGCAGTCTGACCTCTACAC |
| | | TCTGAGCAGCTCAGTGACTGTCCCCTCCAGCACCTGGCCCAGCGAGACCGTCACCTGCAA |
| | | CGTTGCCCACCCGGCCAGCAGCACCAAGGTGGACAAGAAAATTGTGCCCAGGGATTGTG |
| | | GTTGTAAGCCTTGCATATGTACCGTCCCAGAAGTATCATCTGTCTTCATCTTCCCCCCAAA |
| | | GCCCAAGGATGTGCTCACCATTACTCTGACTCCTAAGGTCACGTGTGTTGTGGTAGACAT |
| | | CAGCAAGGATGATCCCGAGGTCCAGTTCAGCTGGTTTGTAGATGATGTGGAGGTGCACA |
| | | CAGCTCAGACGCAACCCCGGGAGGAGCAGTTCAACAGCACTTTCCGCTCAGTCAGTGAA |
| | | CTTCCCATCATGCACCAGGACTGGCTCAATGGCAAGGAGTTCAAATGCAGGGTCAACAG |
| | | TGCAGCTTTCCCTGCCCCCATCGAGAAAACCATCTCCAAAACCAAAGGCAGACCGAAGGC |
| | | TCCACAGGTGTATACCATTCCACCTCCCAAGGAGCAGATGGCCAAGGATAAAGTCAGTCT |
| | | GACCTGCATGATAACAGACTTCTTCCCTGAAGACATTACTGTGGAGTGGCAGTGGAATG |
| | | GGCAGCCAGCGGAGAACTACAAGAACACTCAGCCCATCATGGACACAGATGGCTCTTAC |
| | | TTCGTCTACAGCAAGCTCAATGTGCAGAAGAGCAACTGGGAGGCAGGAAATACTTTCAC |
| | | CTGCTCTGTGTTACATGAGGGCCTGCACAACCACCATACTGAGAAGAGCCTCTCCCACTC |
| | | TCCTGGTAAA |
| 88 | OX86 VL | DIVMTQGALPNPVPSGESASITCRSSQSLVYKDGQTYLNWFLQRPGQSPQLLTYWMSTRAS |
| | | GVSDRFSGSGSGTYFTLKISRVRAEDAGVYYCQQVREYPFTFGSGTKLEIK |
| 89 | OX86 light chain | DIVMTQGALPNPVPSGESASITCRSSQSLVYKDGQTYLNWFLQRPGQSPQLLTYWMSTRAS |
| | | GVSDRFSGSGSGTYFTLKISRVRAEDAGVYYCQQVREYPFTFGSGTKLEIKRADAAPTVSIF |
| | | PPSSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 90 | OX86 light chain | GATATTGTGATGACCCAGGGTGCACTCCCCAATCCTGTCCCTTCTGGAGAGTCAGCTTCC |
| | DNA | ATCACCTGCAGGTCTAGTCAGAGTCTGGTATACAAAGACGGCCAGACATACTTGAATTGG |
| | | TTTCTGCAGAGGCCAGGACAGTCTCCTCAGCTTCTGACCTATTGGATGTCTACCCGTGCAT |
| | | CAGGAGTCTCAGACAGGTTCAGTGGCAGTGGGTCAGGAACATATTTCACACTGAAAATC |
| | | AGTAGAGTGAGGGCTGAGGATGCGGGTGTGTATTACTGTCAGCAAGTTCGAGAGTATCC |
| | | TTTCACTTTCGGCTCAGGGACGAAGTTGGAAATAAAACGGGCTGATGCGGCGCCAACTG |
| | | TATCCATCTTCCCACCATCCAGTGAGCAGTTAACATCTGGAGGTGCCTCAGTCGTGTGCTT |
| | | CTTGAACAACTTCTACCCCAAAGACATCAATGTCAAGTGGAAGATTGATGGCAGTGAACG |
| | | ACAAAATGGCGTCCTGAACAGTTGGACTGATCAGGACAGCAAAGACAGCACCTACAGCA |
| | | TGAGCAGCACCCTCACGTTGACCAAGGACGAGTATGAACGACATAACAGCTATACCTGT |
| | | GAGGCCACTCACAAGACATCAACTTCACCCATTGTCAAGAGCTTCAACAGGAATGAGTGT |
| 91 | Human OX40 | MCVGARRLGRGPCAALLLLGLGLSTVTGLHCVGDTYPSNDRCCHECRPGNGMVSRCSRSQ |
| | | NTVCRPCGPGFYNDVVSSKPCKPCTWCNLRSGSERKQLCTATQDTVCRCRAGTQPLDSYKP |
| | | GVDCAPCPPGHFSPGDNQACKPWTNCTLAGKHTLQPASNSSDAICEDRDPPATQPQETQG |
| | | PPARPITVQPTEAWPRTSQGPSTRPVEVPGGRAVAAILGLGLVLGLLGPLAILLALYLLRR |
| | | DQRLPPDAHKPPGGGSFRTPIQEEQADAHSTLAKI |
| 92 | Mouse OX40 | MYVWVQQPTALLLLGLTLGVTARRLNCVKHTYPSGHKCCRECQPGHGMVSRCDHTRDTLC |
| | | HPCETGFYNEAVNYDTCKQCTQCNHRSGSELKQNCTPTQDTVCRCRPGTQPRQDSGYKLG |
| | | VDCVPCPPGHFSPGNNQACKPWTNCTLSGKQTRHPASDSLDAVCEDRSLLATLLWETQRPT |
| | | FRPTTVQSTTVWPRTSELPSPPTLVTPEGPAFAVLLGLGLGLLAPLTVLLALYLLRKAWRL |
| | | PNTPKPCWGNSFRTPIQEEHTDAHFTLAKI |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of enhancing chemotherapy or radiotherapy efficacy in a subject having a tumor, the method comprising administering to a subject an OX40 agonist and an anti-CTLA4 antibody before, during or after chemotherapy or radiotherapy.

2. A method of treating a subject having a tumor, the method comprising:
(a) administering to the subject an OX40 agonist and an anti-CTLA4 antibody;
(b) obtaining a measurement of cells that indicates a reduction in macrophage differentiation in the subject; and
(c) administering chemotherapy or radiotherapy to the subject.

3. A method of treating a subject having a tumor, the method comprising:
(a) administering to the subject an OX40 agonist and an anti-CTLA4 antibody;
(b) obtaining a measurement of cells that indicates a reduction in macrophage differentiation in the subject; and
(c) administering an anti-IL4 antibody and chemotherapy or radiotherapy to the subject.

4. A method of treating a subject having a tumor, the method comprising:
(a) administering to the subject an OX40 agonist and an anti-CTLA4 antibody;
(b) obtaining a measurement of cells that indicates a reduction in macrophage differentiation in the subject;
(c) administering chemotherapy to the subject;
(d) administering to the subject an OX40 agonist and an anti-CTLA4 antibody; and
(e) administering chemotherapy or radiotherapy to the subject.

5. The method of claim 4, wherein steps (b) and (d) additionally comprise co-administration of an anti-IL-4 antibody.

6. The method of any one of claims 1-5, wherein the subject is identified as having a chemoresistant or radiotherapy resistant tumor.

7. The method of any one of claims 1-6, wherein the method delays or reduces tumor growth, reduces tumor size, and/or enhances survival in the subject.

8. The method of any one of claims 1-7, wherein the tumor is chemoresistant or radiotherapy resistant.

9. The method of any one of claims 1-8, wherein the tumor is non-immunogenic or poorly immunogenic.

10. The method of claim 9, wherein the tumor has poor infiltration of CD8 T cells.

11. The method of any one of claims 1-10, wherein the subject has pancreatic cancer or pancreatic adenocarcinoma.

12. The method of any one of claims 1-11, wherein the tumor is a pancreatic cancer or pancreatic adenocarcinoma.

13. The method of any one of claims 1-12, wherein the chemotherapy comprises administering gemcitabine.

14. The method of any one of claims 1-13, wherein the OX40 agonist is an anti-OX40 antibody.

15. The method of claim 14, wherein the anti-OX40 antibody is one or more of OX86, humanized anti-OX40 antibody, and 9B12.

16. The method of any one of claims 1-15, wherein the OX40 agonist is an OX40 fusion protein.

17. The method of any one of claims 1-16, wherein the anti-CTLA4 antibody is one or more of 9D9 and tremelimumab.

18. The method of any one of claims 1-5, wherein said therapy is administered when immune cell differentiation is reduced in the tumor environment.

19. The method of claim 18, wherein the immune cell is one or more of a macrophage or T cell.

20. The method of claim 19, wherein a reduction in macrophage differentiation is determined by a decrease in arginase expression in a macrophage.

21. The method of any of claims 1-5, wherein the chemotherapy or radiotherapy is administered 1, 2, 3, 4, 5, or 6 days after administration of the OX40 agonist and the anti-CTLA4 antibody.

22. The method of either of claim 3 or 5, wherein the anti-IL4 antibody reduces CD4 T cell differentiation in the tumor environment.

23. The method of any one of claims 1-21, comprising administering the OX40 agonist, the anti-CTLA4 antibody, and said therapy to the subject two or more times.

24. The method of claim 1, comprising administering the OX40 agonist and anti-CTLA4 antibody before chemotherapy.

25. The method of claim 1, comprising administering the OX40 agonist and anti-CTLA4 antibody before radiotherapy.

26. The method of any one of claims 1-11, wherein the subject has colorectal cancer.

27. A method of enhancing chemotherapy or radiotherapy efficacy in a subject having a colorectal cancer, the method comprising administering to the subject an anti-CTLA4 antibody before, during or after chemotherapy or radiotherapy.

28. A method of treating a subject having a colorectal cancer, the method comprising:
(a) administering to the subject an anti-CTLA4 antibody; and
(b) administering radiotherapy to the subject.

29. The method of claim 27 or 28, wherein the anti-CTLA4 antibody is one or more of 9D9 and tremelimumab.

30. The method of any of claims 27-29, wherein the chemotherapy or radiotherapy is administered 1, 2, 3, 4, 5, 6, or 7 days after administration of the anti-CTLA4 antibody.

31. The method of any of claims 27-29, wherein the chemotherapy or radiotherapy is administered 1, 2, 3, or 4 days before administration of the anti-CTLA4 antibody.

32. A method of enhancing chemotherapy or radiotherapy efficacy in a subject having a colorectal cancer, the method comprising administering to a subject an OX40 agonist before, during or after chemotherapy or radiotherapy.

33. A method of treating a subject having a colorectal cancer, the method comprising:
(a) administering radiotherapy to the subject; and
(b) administering to the subject an OX40 agonist.

34. The method of 32 or 33, wherein the OX40 agonist is an anti-OX40 antibody.

35. The method of claim 34, wherein the anti-OX40 antibody is one or more of OX86, humanized anti-OX40 antibody, and 9B12.

36. The method of 32 or 33, wherein the OX40 agonist is an Ox40 fusion protein.

37. The method of any of claims 32-36, wherein the OX40 agonist is administered 1 or 2 days after administration of chemotherapy or radiotherapy.

38. The method of any of claims 27-37, wherein the subject has a colorectal tumor.

39. The method of any one of claims 27-37, wherein the method delays or reduces tumor growth, reduces tumor size, and/or enhances survival in the subject.


SEQUENCE LISTING (108 sequences)

SEQ ID NO: 1 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSKLHSGVPSRFSGSGSRTDYSLTITDLDQEDIATYFCQQGSALPWTFGQGTKVEIK

SEQ ID NO: 2 - protein, 11 aa, Artificial Sequence
  Description of Artificial Sequence Synthetic peptide
RASQDISNYLN

SEQ ID NO: 3 - protein, 7 aa, Artificial Sequence
  Description of Artificial Sequence Synthetic peptide
YTSKLHS

SEQ ID NO: 4 - protein, 9 aa, Artificial Sequence
  Description of Artificial Sequence Synthetic peptide
QQGSALPWT

SEQ ID NO: 5 - protein, 121 aa, Artificial Sequence
  Description of Artificial Sequence Synthetic polypeptide
EVQLQESGPSLVKPSQTLSLTCSVTGDSFTSGYWNWIRKFPGNRLEYMGYISYNGITYHNPSLKSRISITRDTSKNHYYLQLNSVTTEDTATYFCARYRYDYDGGHAMDYWGQGTLVTVSS

SEQ ID NO: 6 - protein, 30 aa, Artificial Sequence
  Description of Artificial Sequence Synthetic polypeptide
QVQLQESGPGLVKPSQTLSLTCAVYGGSFS

SEQ ID NO: 7 - protein, 30 aa, Artificial Sequence
  Description of Artificial Sequence Synthetic polypeptide
QVQLQESGPGLVKPSQTLSLTCAVYGDSFS

SEQ ID NO: 8 - protein, 5 aa, Artificial Sequence
  Description of Artificial Sequence Synthetic peptide
SGYWN

SEQ ID NO: 9 - protein, 14 aa, Artificial Sequence
  Description of Artificial Sequence Synthetic peptide
  MOD_RES (4)..(4): Gln or Lys
  MOD_RES (12)..(12): Trp or Tyr
  MOD_RES (13)..(13): Ile or Met
WIRXHPGKGLEXXG

SEQ ID NO: 10 - protein, 14 aa, Artificial Sequence
  Description of Artificial Sequence Synthetic peptide
WIRQHPGKGLEWIG

SEQ ID NO: 11 - protein, 14 aa, Artificial Sequence
  Description of Artificial Sequence Synthetic peptide
WIRKHPGKGLEYMG

SEQ ID NO: 12 - protein, 14 aa, Artificial Sequence
  Description of Artificial Sequence Synthetic peptide
WIRKHPGKGLEWIG

SEQ ID NO: 13 - protein, 14 aa, Artificial Sequence
  Description of Artificial Sequence Synthetic peptide
WIRKHPGKGLEYIG

SEQ ID NO: 14 - protein, 16 aa, Artificial Sequence
  Description of Artificial Sequence Synthetic peptide
YISYNGITYHNPSLKS

SEQ ID NO: 15 - protein, 16 aa, Artificial Sequence
  Description of Artificial Sequence Synthetic peptide
YISYNAITYHNPSLKS

SEQ ID NO: 16 - protein, 16 aa, Artificial Sequence
  Description of Artificial Sequence Synthetic peptide
YISYSGITYHNPSLKS

SEQ ID NO: 17 - protein, 32 aa, Artificial Sequence
  Description of Artificial Sequence Synthetic polypeptide
  MOD_RES (6)..(6): Pro or Arg
  MOD_RES (13)..(13): Phe or Tyr
  MOD_RES (29)..(29): Tyr or Phe
RITINXDTSKNQXSLQLNSVTPEDTAVYXCAR

SEQ ID NO: 18 - protein, 32 aa, Artificial Sequence
  Description of Artificial Sequence Synthetic polypeptide
RITINPDTSKNQFSLQLNSVTPEDTAVYYCAR

SEQ ID NO: 19 - protein, 32 aa, Artificial Sequence
  Description of Artificial Sequence Synthetic polypeptide
RITINRDTSKNQYSLQLNSVTPEDTAVYFCAR

SEQ ID NO: 20 - protein, 32 aa, Artificial Sequence
  Description of Artificial Sequence Synthetic polypeptide
RITINRDTSKNQFSLQLNSVTPEDTAVYYCAR

SEQ ID NO: 21 - protein, 32 aa, Artificial Sequence
  Description of Artificial Sequence Synthetic polypeptide
RITINRDTSKNQFSLQLNSVTPEDTAVYFCAR

SEQ ID NO: 22 - protein, 32 aa, Artificial Sequence
  Description of Artificial Sequence Synthetic polypeptide
RITINRDTSKNQYSLQLNSVTPEDTAVYYCAR

SEQ ID NO: 23 - protein, 32 aa, Artificial Sequence
  Description of Artificial Sequence Synthetic polypeptide
RITINPDTSKNQYSLQLNSVTPEDTAVYFCAR

SEQ ID NO: 24 - protein, 32 aa, Artificial Sequence
  Description of Artificial Sequence Synthetic polypeptide
RITINPDTSKNQYSLQLNSVTPEDTAVYYCAR

SEQ ID NO: 25 - protein, 13 aa, Artificial Sequence
  Description of Artificial Sequence Synthetic peptide
YRYDYDGGHAMDY

SEQ ID NO: 26 - protein, 13 aa, Artificial Sequence
  Description of Artificial Sequence Synthetic peptide
YKYDYDAGHAMDY

SEQ ID NO: 27 - protein, 13 aa, Artificial Sequence
  Description of Artificial Sequence Synthetic peptide
YKYDYDGGHAMDY

SEQ ID NO: 28 - protein, 11 aa, Artificial Sequence
  Description of Artificial Sequence Synthetic peptide
WGQGTLVTVSS

SEQ ID NO: 29 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 30 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 642 bp, Artificial Sequence
  Description of Artificial Sequence Synthetic polynucleotide
gacatccagatgacccagagccccagcagcctgagcgccagcgtgggcgacagagtgaccatcacctgtcgggccagccaggacatcagcaactacctgaactggtatcagcagaagcccggcaaggcccccaagctgctgatctactacaccagcaagctgcacagcggcgtgcccagcagattcagcggcagcggctccggcaccgactacaccctgaccatcagcagcctgcagcccgaggacttcgccacctactactgccagcagggctccgccctgccctggacctttggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 32 - protein, 107 aa, Artificial Sequence
  Description of Artificial Sequence Synthetic polypeptide
DIQMTQSPSSLSASVGDRVTITCRASQDISNYLNWYQQKPGKAVKLLIYYTSKLHSGVPSRFSGSGSRTDYTLTISSLQPEDFATYYCQQGSALPWTFGQGTKVEIK

SEQ ID NO: 33 - protein, 121 aa, Artificial Sequence
  Description of Artificial Sequence Synthetic polypeptide
QVQLQESGPGLVKPSQTLSLTCAVYGGSFSSGYWNWIRQHPGKGLEWIGYISYNGITYHNPSLKSRITINPDTSKNQFSLQLNSVTPEDTAVYYCARYRYDYDGGHAMDYWGQGTLVTVSS

SEQ ID NO: 34 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggcagcaccccggcaagggcctggaatggatcggctacatcagctacaacggcatcacctaccacaaccccagcctgaagtcccggatcaccatcaaccccgacaccagcaagaaccagttctccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 35 - protein, 121 aa, Artificial Sequence
  Description of Artificial Sequence Synthetic polypeptide
QVQLQESGPGLVKPSQTLSLTCAVYGGSFSSGYWNWIRKHPGKGLEWIGYISYNGITYHNPSLKSRITINRDTSKNQFSLQLNSVTPEDTAVYYCARYRYDYDGGHAMDYWGQGTLVTVSS

SEQ ID NO: 36 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatggatcggctacatcagctacaacggcatcacctaccacaaccccagcctgaagtcccggatcaccatcaaccgggacaccagcaagaaccagttctccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 37 - protein, 121 aa, Artificial Sequence
  Description of Artificial Sequence Synthetic polypeptide
QVQLQESGPGLVKPSQTLSLTCAVYGDSFSSGYWNWIRKHPGKGLEYMGYISYNGITYHNPSLKSRITINRDTSKNQYSLQLNSVTPEDTAVYFCARYRYDYDGGHAMDYWGQGTLVTVSS

SEQ ID NO: 38 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcgacagcttcagcagcggctactggaactggatccggaagcaccccggcaagggcctggaatacatgggctacatcagctacaacggcatcacctaccacaaccccagcctgaagtcccggatcaccatcaaccgggacaccagcaagaaccagtactccctgcagctgaacagcgtgacccccgaggacaccgccgtgtacttctgcgcccggtacagatacgactacgacggcggccacgccatggactactggggccagggcaccctggtcaccgtgtcctct

SEQ ID NO: 39 - protein, 121 aa, Artificial Sequence
  Description of Artificial Sequence Synthetic polypeptide
QVQLQESGPGLVKPSQTLSLTCAVYGDSFSSGYWNWIRKHPGKGLEYIGYISYNGITYHNPSLKSRITINRDTSKNQYSLQLNSVTPEDTAVYFCARYRYDYDGGHAMDYWGQGTLVTVSS

SEQ ID NO: 40 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcgacagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtacttctgcgcccggtacagatacgactacgacggcggccacgccatggactactggggccagggcaccctggtcaccgtgtcctct

SEQ ID NO: 41 - protein, 121 aa, Artificial Sequence
  Description of Artificial Sequence Synthetic polypeptide
QVQLQESGPGLVKPSQTLSLTCAVYGDSFSSGYWNWIRKHPGKGLEYIGYISYNGITYHNPSLKSRITINRDTSKNQFSLQLNSVTPEDTAVYFCARYRYDYDGGHAMDYWGQGTLVTVSS

SEQ ID NO: 42 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcgacagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagttctccctgcagctgaacagcgtgacccccgaggacaccgccgtgtacttctgcgcccggtacagatacgactacgacggcggccacgccatggactactggggccagggcaccctggtcaccgtgtcctct

SEQ ID NO: 43 - protein, 121 aa, Artificial Sequence
  Description of Artificial Sequence Synthetic polypeptide
QVQLQESGPGLVKPSQTLSLTCAVYGDSFSSGYWNWIRKHPGKGLEYIGYISYNGITYHNPSLKSRITINRDTSKNQYSLQLNSVTPEDTAVYYCARYRYDYDGGHAMDYWGQGTLVTVSS

SEQ ID NO: 44 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcgacagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 45 - protein, 121 aa, Artificial Sequence
  Description of Artificial Sequence Synthetic polypeptide
QVQLQESGPGLVKPSQTLSLTCAVYGDSFSSGYWNWIRKHPGKGLEYIGYISYNGITYHNPSLKSRITINRDTSKNQFSLQLNSVTPEDTAVYYCARYRYDYDGGHAMDYWGQGTLVTVSS

SEQ ID NO: 46 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcgacagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagttctccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 47 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINPDTSKNQYSLQLNSVTPEDTAVYFCARYRYDYDGGHAMDYWGQGTLVTVSS

SEQ ID NO: 48 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccccgacaccagcaagaaccagtactccctgcagctgaacagcgtgacccccgaggacaccgccgtgtacttctgcgcccggtacagatacgactacgacggcggccacgccatggactactggggccagggcaccctggtcaccgtgtcctct

SEQ ID NO: 49 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYFCARYRYDYDGGHAMDYWGQGTLVTVSS

SEQ ID NO: 50 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtacttctgcgcccggtacagatacgactacgacggcggccacgccatggactactggggccagggcaccctggtcaccgtgtcctct

SEQ ID NO: 51 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYRYDYDGGHAMDYWGQGTLVTVSS

SEQ ID NO: 52 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 53 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINPDTSKNQYSLQLNSVTPEDTAVYYCARYRYDYDGGHAMDYWGQGTLVTVSS

SEQ ID NO: 54 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccccgacaccagcaagaaccagtactccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 55 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNAITYHNPSLKSRITINRDTSKNQYSLQLNSVTPEDTAVYYCARYKYDYDAGHAMDYWGQGTLVTVSS

SEQ ID NO: 56 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacgccatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacgccggccacgccatggactactggggccagggcaccctggtcaccgtgtcctct

SEQ ID NO: 57 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNAITYHNPSLKSRITINRDTSKNQYSLQLNSVTPEDTAVYYCARYRYDYDGGHAMDYWGQGTLVTVSS

SEQ ID NO: 58 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacgccatcacctaccacaaccccagcctgaagtcccggatcaccatcaaccgggacaccagcaagaaccagtactccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 59 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 60 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctct

SEQ ID NO: 61 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYSGITYHNPSLKSRITINRDTSKNQYSLQLNSVTPEDTAVYYCARYRYDYDGGHAMDYWGQGTLVTVSS

SEQ ID NO: 62 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacagcggcatcacctaccacaaccccagcctgaagtcccggatcaccatcaaccgggacaccagcaagaaccagtactccctgcagctgaacagcgtgacccccgaggacaccgccgtgtactactgcgcccggtacagatacgactacgacggcggccacgccatggactactggggccagggcaccctggtcaccgtgtcctct

SEQ ID NO: 63 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYSGITYHNPSLKSRITINRDTSKNQYSLQLNSVTPEDTAVYYCARYKYDYDGGHAMDYWGQGTLVTVSS

SEQ ID NO: 64 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacagcggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctct

SEQ ID NO: 65 - protein, 121 aa, Artificial Sequence
  Description of Artificial Sequence Synthetic polypeptide
EVQLQESGPSLVKPSQTLSLTCSVTGDSFTSGYWNWIRKFPGNRLEYMGYISYNAITYHNPSLKSRISITRDTSKNHYYLQLNSVTTEDTATYFCARYRYDYDGGHAMDYWGQGTLVTVSS

SEQ ID NO: 66 - DNA, 363 bp, Artificial Sequence
  Description of Artificial Sequence Synthetic polynucleotide
gaggtgcagctgcaggaaagcggccccagcctggtcaagcccagccagaccctgagcctgacctgcagcgtgaccggcgacagcttcaccagcggctactggaactggatccggaagttccccggcaaccggctcgagtacatgggctacatcagctacaacgccatcacctaccacaaccccagcctgaagtcccggatcagcatcacccgggacaccagcaagaaccactactacctgcagctgaacagcgtgaccaccgaggacaccgccacctacttttgcgcccggtacagatacgactacgacggcggccacgccatggactactggggccagggcaccctggtcaccgtgtcctct

SEQ ID NO: 67 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNAITYHNPSLKSRITINRDTSKNQYSLQLNSVTPEDTAVYYCARYKYDYDGGHAMDYWGQGTLVTVSS

SEQ ID NO: 68 - DNA, 363 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacgccatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctct

SEQ ID NO: 69 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 990 bp, Homo sapiens
gcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 71 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1353 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 73 - protein, 448 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - DNA, 1344 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgtcgaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacctgcaacgtggaccacaagcccagcaacaccaaggtggacaagagagtggagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaag

SEQ ID NO: 75 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1353 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 77 - protein, 448 aa, Artificial Sequence
  Description of Artificial Sequence Synthetic polypeptide
QVQLQESGPGLVKPSQTLSLTCAVYGGSFSSGYWNWIRKHPGKGLEYIGYISYNAITYHNPSLKSRITINRDTSKNQYSLQLNSVTPEDTAVYYCARYKYDYDGGHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - DNA, 1344 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacgccatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgtcgaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacctgcaacgtggaccacaagcccagcaacaccaaggtggacaagagagtggagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaag

SEQ ID NO: 79 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNAITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 1353 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacgccatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 81 - protein, 445 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 82 - DNA, 1335 bp, Artificial Sequence
  Description of Artificial Sequence Synthetic polynucleotide
caggtgcagctgcaggaaagcggccctggcctggtcaagcccagccagaccctgagcctgacctgtgccgtgtacggcggcagcttcagcagcggctactggaactggatccggaagcaccccggcaagggcctggaatacatcggctacatcagctacaacggcatcacctaccacaaccccagcctgaagtcccggatcaccatcaaccgggacaccagcaagaaccagtactccctgcagctgaacagcgtgacccccgaggacaccgccgtgtactactgcgcccggtacaaatacgactacgacggcggccacgccatggactactggggccagggcaccctggtcaccgtgtcctctgcgaagacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtaccgtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtataccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 83 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 84 - DNA, 642 bp, Artificial Sequence
  Description of Artificial Sequence Synthetic polynucleotide
gacatccagatgacccagagccccagcagcctgagcgccagcgtgggcgacagagtgaccatcacctgtcgggccagccaggacatcagcaactacctgaactggtatcagcagaagcccggcaaggcccccaagctgctgatctactacaccagcaagctgcacagcggcgtgcccagcagattcagcggcagcggctccggcaccgactacaccctgaccatcagcagcctgcagcccgaggacttcgccacctactactgccagcagggctccgccctgccctggacctttggccagggcaccaaggtggaaatcaagcgggctgatgcggcgccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 85 - protein, 117 aa, Artificial Sequence
  Description of Artificial Sequence Synthetic polypeptide
QVQLKESGPGLVQPSQTLSLTCTVSGFSLTGYNLHWVRQPPGKGLEWMGRMRYDGDTYYNSVLKSRLSISRDTSKNQVFLKMNSLQTDDTAIYYCTRDGRGDSFDYWGQGVMVTVSS

SEQ ID NO: 86 - protein, 441 aa, Artificial Sequence
  Description of Artificial Sequence Synthetic polypeptide
QVQLKESGPGLVQPSQTLSLTCTVSGFSLTGYNLHWVRQPPGKGLEWMGRMRYDGDTYYNSVLKSRLSISRDTSKNQVFLKMNSLQTDDTAIYYCTRDGRGDSFDYWGQGVMVTVSSASTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 87 - DNA, 1323 bp, Artificial Sequence
  Description of Artificial Sequence Synthetic polynucleotide
caggtgcagctgaaggagtcaggacctggtctggtgcagccctcacagaccctgtccctcacctgcactgtctctgggttctcactaaccggttacaatttacactgggttcgccagcctccaggaaagggtctggagtggatgggaagaatgaggtatgatggagacacatattataattcagttctcaaatcccgactgagcatcagcagggacacctccaagaaccaagttttcttgaaaatgaacagtctgcaaacggatgacacagccatttactattgtaccagagacgggcgtggtgactcctttgattactggggccaaggagtcatggtcacagtctcctccgcgtcgacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtaccgtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtataccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 88 - protein, 112 aa, Artificial Sequence
  Description of Artificial Sequence Synthetic polypeptide
DIVMTQGALPNPVPSGESASITCRSSQSLVYKDGQTYLNWFLQRPGQSPQLLTYWMSTRASGVSDRFSGSGSGTYFTLKISRVRAEDAGVYYCQQVREYPFTFGSGTKLEIK

SEQ ID NO: 89 - protein, 219 aa, Artificial Sequence
  Description of Artificial Sequence Synthetic polypeptide
DIVMTQGALPNPVPSGESASITCRSSQSLVYKDGQTYLNWFLQRPGQSPQLLTYWMSTRASGVSDRFSGSGSGTYFTLKISRVRAEDAGVYYCQQVREYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 90 - DNA, 657 bp, Artificial Sequence
  Description of Artificial Sequence Synthetic polynucleotide
gatattgtgatgacccagggtgcactccccaatcctgtcccttctggagagtcagcttccatcacctgcaggtctagtcagagtctggtatacaaagacggccagacatacttgaattggtttctgcagaggccaggacagtctcctcagcttctgacctattggatgtctacccgtgcatcaggagtctcagacaggttcagtggcagtgggtcaggaacatatttcacactgaaaatcagtagagtgagggctgaggatgcgggtgtgtattactgtcagcaagttcgagagtatcctttcactttcggctcagggacgaagttggaaataaaacgggctgatgcggcgccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 91 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 92 - protein, 272 aa, Mus sp.
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 93 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 94 - protein, 153 aa, Homo sapiens [sequence omitted]

SEQ ID NO: 95 - protein, 412 aa, Artificial Sequence
  Description of Artificial Sequence Synthetic polypeptide
APLATDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKELLGGGSIKQIEDKIEEILSKIYHIENEIARIKKLIGERGHGGGSNSQVSHRYPRFQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 96 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 97 - DNA, 1206 bp, Artificial Sequence
  Description of Artificial Sequence Synthetic polynucleotide
gagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaaggaccaggataagatcgaggctctgtcctccaaggtgcagcagctggaacggtccatcggcctgaaggacctggccatggctgacctggaacagaaagtgctggaaatggaagcctccacacaggtgtcacacagatacccccggatccagtccattaaggtgcagttcaccgagtacaagaaagagaagggctttatcctgacctcccagaaagaggacgagatcatgaaggtgcagaacaactccgtgatcatcaactgcgacgggttctacctgatctccctgaagggctacttcagccaggaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacacctccctggacgacttccacgtgaacggcggcgagctgatcctgatccaccagaaccctggcgagttctgcgtgctg

SEQ ID NO: 98 - protein, 402 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 99 - DNA, 1206 bp, Artificial Sequence
  Description of Artificial Sequence Synthetic polynucleotide
gagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaaggaccaggataagatcgaggctctgtcctccaaggtgcagcagctggaacggtccatcggcctgaaggacctggccatggctgacctggaacagaaagtgctggaaatggaagcctccacacaggtgtcacacagatacccccggatccagtccattaaggtgcagttcaccgagtacaagaaagagaagggctttatcctgacctcccagaaagaggacgagatcatgaaggtgcagaacaactccgtgatcatcaactgcgacgggttctacctgatctccctgaagggctacttcagccaggaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacacctccctggacgacttccacgtgaacggcggcgagctgatcctgatccaccagaaccctggcgaggcctgcgtgctg

SEQ ID NO: 100 - protein, 402 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEACVL

SEQ ID NO: 101 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 102 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 103 - protein, 10 aa, Artificial Sequence
  Description of Artificial Sequence Synthetic peptide
GFTFSSYGMH

SEQ ID NO: 104 - protein, 15 aa, Artificial Sequence
  Description of Artificial Sequence Synthetic peptide
VIWYDGSNKYYADSV

SEQ ID NO: 105 - protein, 16 aa, Artificial Sequence
  Description of Artificial Sequence Synthetic peptide
DPRGATLYYYYYGMDV

SEQ ID NO: 106 - protein, 11 aa, Artificial Sequence
  Description of Artificial Sequence Synthetic peptide
RASQSINSYLD

SEQ ID NO: 107 - protein, 7 aa, Artificial Sequence
  Description of Artificial Sequence Synthetic peptide
AASSLQS

SEQ ID NO: 108 - protein, 9 aa, Artificial Sequence
  Description of Artificial Sequence Synthetic peptide
QQYYSTPFT